SEQUENCE LISTING (17 sequences)

SEQ ID NO: 1 - RNA, 36 nt, synthetic construct
  source 1..36: mol_type = other RNA
taatttctaagtgtagatgggggttggtagggtgtc

SEQ ID NO: 2 - DNA, 27 bp, synthetic construct
  source 1..27: mol_type = other DNA
gaagacaccctaccaaccccctaaacc

SEQ ID NO: 3 - DNA, 21 bp, synthetic construct
  source 1..21: mol_type = other DNA
gaagacaccctaccaaccccc

SEQ ID NO: 4 - DNA, 20 bp, synthetic construct
  source 1..20: mol_type = other DNA
ttattttttttattttttat

SEQ ID NO: 5 - DNA, 24 bp, synthetic construct
  source 1..24: mol_type = other DNA
gaagacaccctaccaacccccccc

SEQ ID NO: 6 - RNA, 42 nt, synthetic construct
  source 1..42: mol_type = other RNA
taatttctactaagtgtagatggggggggttggtagggtgtc

SEQ ID NO: 7 - DNA, 20 bp, synthetic construct
  source 1..20: mol_type = other DNA
tcgtttagggcgtggactac

SEQ ID NO: 8 - DNA, 20 bp, synthetic construct
  source 1..20: mol_type = other DNA
ggcgacctgctggaacatta

SEQ ID NO: 9 - RNA, 45 nt, synthetic construct
  source 1..45: mol_type = other RNA
taatttctactaagtgtagatgcgcaatcagcgtcagtaatgttc

SEQ ID NO: 10 - DNA, 19 bp, synthetic construct
  source 1..19: mol_type = other DNA
cagaatggagaacgcagtg

SEQ ID NO: 11 - DNA, 20 bp, synthetic construct
  source 1..20: mol_type = other DNA
gtgaaccaagacgcagtatt

SEQ ID NO: 12 - RNA, 42 nt, synthetic construct
  source 1..42: mol_type = other RNA
taatttctactaagtgtagatcccaataatactgcgtcttgg

SEQ ID NO: 13 - RNA, 50 nt, synthetic construct
  source 1..50: mol_type = other RNA
ggaccaccccaaaaacgaaggggactaaaacagagaattccatagtccag

SEQ ID NO: 14 - RNA, 32 nt, synthetic construct
  source 1..32: mol_type = other RNA
ggactggactatggaattctcgggtgccaagg

SEQ ID NO: 15 - RNA, 60 nt, synthetic construct
  source 1..60: mol_type = other RNA
ggggatttagactaccccaaaaacgaaggggactaaaacacgttgttttgatcgcgcccc

SEQ ID NO: 16 - RNA, 80 nt, synthetic construct
  source 1..80: mol_type = other RNA
cagaatggagaacgcagtggggcgcgatcaaaacaacgtcggccccaaggtttacccaataatactgcgtcttggttcac

SEQ ID NO: 17 - DNA, 30 bp, synthetic construct
  source 1..30: mol_type = other DNA
gaagacaccctaccaacccccccctaaacc

(19) United States
(12) Patent Application Publication
(10) Pub. No.: US 2024/0426838 A1
(43) Pub. Date: Dec. 26, 2024

(54) CRISPR/CAS-Associated Detection Assays, Methods and Kits

(71) Applicants: BioPoint Pty Limited, Belrose, New South Wales (AU); NewSouth Innovations Pty Limited, University of New South Wales, New South (AU)

(72) Inventors: Graham Vesey, Belrose, New South Wales (AU); Ewa Goldys, University of New South Wales, New South (AU); Fei Deng, University of New South Wales, New South (AU); Yi Li, University of New South Wales, New South (AU); Guozhen Liu, University of New South Wales, New South (AU)

(21) Appl. No.: 18/684,044

(22) PCT Filed: Aug. 2, 2022
(86) PCT No.: PCT/AU2022/050829

§ 371 Date: Feb. 15, 2024

(30) Foreign Application Priority Data
Aug. 19, 2021 (AU) 2021902608

(51) Int. Cl. G01N 33/68; C12N 9/22; C12N 15/11; G01N 33/53

(52) U.S. Cl. CPC G01N 33/6866; C12N 9/22; C12N 15/11; G01N 33/5308; C12N 2310/20; G01N 2333/57

(57) ABSTRACT

The present invention relates to CRISPR/Cas-based biosensing materials, assays and methods. In particular the technology relates to ultrasensitive CRISPR/Cas-based methods suitable for microbial detection, detection of cells or cellular components, small molecules, cytokines, polypeptides and various other biomarkers. The materials and methods according to the invention may also be used to enhance the sensitivity of existing bioassays.

### TECHNICAL FIELD

The technology relates to CRISPR/Cas-based biosensing assays and methods. In particular the technology relates to ultrasensitive CRISPR/Cas-based methods suitable for microbial detection, identification and optional recovery of microbes in a sample. The ultrasensitive CRISPR/Cas-based methods are also suitable for the detection of cells or cellular components, small molecules, cytokines, polypeptides and various other biomarkers.

### CROSS REFERENCE TO RELATED APPLICATION

The present application claims priority to Australian provisional patent application No. 2021902608, which was filed on 19 Aug. 2021. The entire content of this application is hereby incorporated herein by reference.

### BACKGROUND

Current antibody conjugates, such as IgG protein conjugates with enzymatic materials, such as HRP or nanozyme which can generate colorimetric or chemiluminescent signals used for signal generation in immunoassays suffer from a sensitivity barrier, with assay detection limits generally in the pM to nM range. This is insufficient in certain biological or clinical diagnostic scenarios such as in emergency medicine or fertility where higher sensitivity is required.

Recent discoveries of programmable nucleases in various CRISPR/Cas systems opened new opportunities for their applications in biosensing. The discovery of unique collateral cleavage by Class 2 type V or type VI CRISPR/Cas proteins, such as Cas12, Cas13 or Cas14, has led to the demonstration of a highly specific and efficient biosensing signal amplification strategy which is enabling ultrasensitive detection.

Shortly after the first reported CRISPR/Cas-based nucleic acid detection in 2016 (East-Seletsky et al., Nature 538(7624) (2016): 270-273), different types of novel biosensing systems using either Cas12a or Cas13a effectors have been established. Their exceptional performance, including attomolar-level sensitivity, single nucleotide specificity, and room temperature reaction, is combined with high specificity and ease of reprogramming.

CRISPR/Cas-based biosensing has now been combined with other types of detection technologies, including electrochemical sensors, SERS (Surface-Enhanced Raman Scattering), and has been employed for biosensing for a wide range of diverse targets, including various proteins, small molecules, and ions. CRISPR/Cas-based biosensing has been utilised for pathogen detection based on their nucleic acid sequences.

However, previously reported approaches generally rely on additional nucleic acid molecules for target recognition or signal amplification. These may lead to the introduction of free DNA oligos or more complex DNA 3D structures into the sensing system, whose integrity can be jeopardized by the nucleases commonly found in various biological samples. This can potentially lead to limited performance in real diagnostic scenarios. There is a need, however, to provide assays which enable highly sensitive detection of analytes, including single whole microorganisms.

Cryptosporidium and Giardia are among the most common enteric parasites of humans. Human infections by Giardia and Cryptosporidium are transmitted via the fecal-oral route, either as a result of person-to-person transmission or through secondary transmission by contaminated food or water. Cryptosporidium is recognized as one of the major causative agents responsible for serious gastrointestinal disorders worldwide. This pathogen is commonly found in various surface water and wastewater sources, and relevant public health controls require effective and efficient water monitoring for their presence.

Water is the most common transmission vehicle reported. These pathogens represent a significant challenge to public health and drinking water suppliers due to their persistence and survival in water, their resistance to some drinking water treatment methods, and their low infectious doses. Furthermore, detecting Giardia cysts and Cryptosporidium oocysts in water is very difficult due to their small size, their relatively low numbers in most waters, the inability to multiply their numbers by in vitro culture, and the difficulty in identifying them from the large numbers of other particles and debris in samples.

The extremely low infectious dose (<10 Cryptosporidium oocysts), microscopic size (˜5 to 10 μm), and the requirement for detection in complex environmental and food samples represent major technical challenges for establishing effective pathogen detection and diagnostics with adequate sensitivity, simplicity and low cost.

The present inventors have developed improved biosensing materials and methods using CRISPR/Cas biosensing technology. These improved materials and methods enable the sensitive detection of various analytes, including whole pathogens, with single-cell sensitivity, and small proteins (e.g. cytokines) with a remarkable assay sensitivity down to 10 fg/mL. The inventors have also developed methods to sensitively detect and recover Giardia cysts or Cryptosporidium oocysts in a sample.

### SUMMARY

According to one aspect the present invention provides a method for the detection of a target in a sample, the method comprising:
(a) contacting the sample with:
(i) a first target binding construct;
(ii) a type V or type VI CRISPR/Cas effector protein;
(iii) a trigger nucleic acid sequence;
(iv) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
(v) a labelled reporter construct, wherein said reporter construct is a nucleic acid that does not hybridize with the guide sequence of the guide RNA and is cleavable by the nuclease activity of the activated type V or type VI CRISPR/Cas effector protein; and
(b) measuring a detectable signal produced by cleavage of the labelled nucleic acid reporter by the type V or type VI CRISPR/Cas effector protein, thereby detecting the target in the sample;
   wherein the type V or type VI CRISPR/Cas effector protein, the trigger nucleic acid, the guide RNA, or the type V or type VI CRISPR/Cas effector protein in combination with the guide RNA optionally in further combination with the trigger nucleic acid, is conjugated to the first target binding construct to thereby co-locate the type V or type VI CRISPR/Cas effector protein and the target when present in the sample; and wherein the target is not a nucleic acid sequence.

According to a second aspect, the invention provides a method for the detection of a target in a sample, the method comprising:
(a) contacting the sample with:
(i) a first target binding construct;
(ii) a second target binding construct to immobilise or capture the target;
(iii) a type V or type VI CRISPR/Cas effector protein;
(iv) a trigger nucleic acid sequence;
(v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
(vi) a labelled reporter construct, wherein said reporter construct is a nucleic acid that does not hybridize with the guide sequence of the guide RNA and is cleavable by the nuclease activity of the activated type V or type VI CRISPR/Cas effector protein; and
(b) measuring a detectable signal produced following cleavage of the labelled nucleic acid reporter by the type V or type VI CRISPR/Cas effector protein, thereby detecting the immobilised or captured target;
   wherein the type V or type VI CRISPR/Cas effector protein, the trigger nucleic acid sequence, the guide RNA, or the type V or type VI CRISPR/Cas effector protein in combination with the guide RNA optionally in further combination with the trigger nucleic acid, is conjugated to the first target binding construct to thereby co-locate the type V or type VI CRISPR/Cas effector protein and the target when present in the sample; and wherein the target is not a nucleic acid sequence.

According to a third aspect, the invention provides a method for the detection of a target in a sample, the method comprising:
(a) contacting the sample with:
(i) a first target binding construct to thereby immobilise or capture the target;
(ii) a second target binding construct;
(iii) a third binding construct which binds to the second target binding construct;
(iv) a type V or type VI CRISPR/Cas effector protein;
(v) a trigger nucleic acid sequence;
(vi) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
(vii) a labelled reporter construct, wherein said reporter construct comprises a nucleic acid that does not hybridize with the guide sequence of the guide RNA and is cleavable by the nuclease activity of the activated type V or type VI CRISPR/Cas effector protein; and
(b) measuring a detectable signal produced following cleavage of the labelled nucleic acid reporter by the type V or type VI CRISPR/Cas effector protein, thereby detecting the immobilised or captured target;
   wherein the type V or type VI CRISPR/Cas effector protein, the trigger nucleic acid sequence, the guide RNA, or the type V or type VI CRISPR/Cas effector protein in combination with the guide RNA optionally in further combination with the trigger nucleic acid, is conjugated to the third binding construct to thereby co-locate the type V or type VI CRISPR/Cas effector protein and the target when present in the sample; and wherein the target is not a nucleic acid sequence.

According to a fourth aspect the invention provides a kit for detecting a target in a sample, the kit comprising:
(i) a first binding construct;
(ii) a type V or type VI CRISPR/Cas effector protein;
(iii) a trigger nucleic acid sequence;
(iv) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
(v) a labelled reporter construct, wherein said reporter construct comprises a nucleic acid, optionally single stranded, that does not hybridize with the guide sequence of the guide RNA and is cleavable by the nuclease activity of the activated type V or type VI CRISPR/Cas effector protein;
   wherein the first binding construct binds to the target or to another agent which binds or attaches to the target, and wherein the type V or type VI CRISPR/Cas effector protein, the trigger nucleic acid sequence, the guide RNA, or the type V or type VI CRISPR/Cas effector protein in combination with the guide RNA optionally in further combination with the trigger nucleic acid, is conjugated to the first binding construct to thereby co-locate the type V or type VI CRISPR/Cas effector protein and the target when present in the sample; and wherein the target is not a nucleic acid sequence.

According to a fifth aspect, the invention provides a kit for detecting a target in a sample, the kit comprising:
(i) a target binding construct;
(ii) a second target binding construct;
(iii) a type V or type VI CRISPR/Cas effector protein;
(iv) a trigger nucleic acid sequence;
(v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
(vi) a labelled reporter construct, wherein said reporter construct comprises a nucleic acid, optionally single stranded, that does not hybridize with the guide sequence of the guide RNA and is cleavable by the nuclease activity of the activated type V or type VI CRISPR/Cas effector protein;
   wherein the type V or type VI CRISPR/Cas effector protein, the trigger nucleic acid sequence, the guide RNA, or the type V or type VI CRISPR/Cas effector protein in combination with the guide RNA optionally in further combination with the trigger nucleic acid, is conjugated to the second target binding construct so as to co-locate the type V or type VI CRISPR/Cas effector protein and the target when present in the sample; and wherein the target is not a nucleic acid sequence.

According to a sixth aspect, the present invention provides a method of enhancing the method of the first second or third aspects comprising adding a sulfhydryl reductant, and/or a non-ionic surfactant.

According to a seventh aspect, the present invention provides a method for the detection of a nucleic acid target in a sample, comprising: (a) contacting the sample with a reaction mixture comprising: (i) a type V CRISPR/Cas effector protein (ii) a guide RNA comprising: a region that binds to the type V or type VI CRISPR/Cas effector protein, and a guide sequence that hybridizes with the target nucleic acid sequence, wherein hybridization between the guide sequence and the target nucleic acid sequence activates the nuclease activity of the CRISPR/Cas effector protein; (iii) a labelled reporter construct, wherein said reporter construct is a single stranded RNA (ssRNA) sequence that does not hybridize with the guide sequence of the guide RNA and is cleavable by the nuclease activity of the activated type V or type VI CRISPR/Cas effector protein; and iv) a sulfhydryl reductant, and/or v) a non-ionic surfactant; and (b) measuring a detectable signal produced following cleavage of the labelled nucleic acid reporter by the type V CRISPR/Cas effector protein, thereby detecting the target in the sample.

Numbered statements of the invention are as follows:
1. A method for the detection of a target in a sample, the method comprising:
   (a) contacting the sample with:
      (i) a first target binding construct;
      (ii) a type V or type VI CRISPR/Cas effector protein;
      (iii) a trigger nucleic acid sequence;
      (v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
      (iv) a labelled reporter construct, wherein said reporter construct comprises a nucleic acid that does not hybridize with the guide sequence of the guide RNA and is cleavable by the nuclease activity of the activated type V or type VI CRISPR/Cas effector protein; and
   (b) measuring a detectable signal produced following cleavage of the labelled nucleic acid reporter by the type V or type VI CRISPR/Cas effector protein, thereby detecting the target in the sample;
   wherein the type V or type VI CRISPR/Cas effector protein, the trigger nucleic acid sequence, the guide RNA, the type V or type VI CRISPR/Cas effector protein in combination with the guide RNA, or the type V or type VI CRISPR/Cas effector protein in combination with the guide RNA and the trigger nucleic acid is linked or conjugated to the first target binding construct to thereby co-locate the type V or type VI CRISPR/Cas effector protein and the target when present in the sample; and wherein the target is not a nucleic acid sequence.
2. A method for the detection of a target in a sample, the method comprising:
   (a) contacting the sample with:
      (i) a first target binding construct;
      (ii) a second target binding construct to immobilise or capture the target;
      (iii) a type V or type VI CRISPR/Cas effector protein;
      (iv) a trigger nucleic acid sequence;
      (v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
      (vi) a labelled reporter construct, wherein said reporter construct comprises a nucleic acid that does not hybridize with the guide sequence of the guide RNA and is cleavable by the nuclease activity of the activated type V or type VI CRISPR/Cas effector protein; and
   (b) measuring a detectable signal produced following cleavage of the labelled nucleic acid reporter by the type V or type VI CRISPR/Cas effector protein, thereby detecting the immobilised or captured target;
   wherein the type V or type VI CRISPR/Cas effector protein, the trigger nucleic acid sequence, the guide RNA, the type V or type VI CRISPR/Cas effector protein in combination with the guide RNA, or the type V or type VI CRISPR/Cas effector protein in combination with the guide RNA and the trigger nucleic acid is linked or conjugated to the first target binding construct to thereby co-locate the type V or type VI CRISPR/Cas effector protein and the target when present in the sample; and wherein the target is not a nucleic acid sequence.
3. The method of statement 1 or 2, wherein the type V or type VI CRISPR/Cas effector protein is a Cas12a (Cpf1) or Cas12b (C2c1) protein, or Cas 13a or Cas 13b.
4. The method of any one of statements 1-3, wherein the reporter construct is a labelled RNA.
5. The method of any one of statements 1-3, wherein the reporter construct is a labelled DNA.
6. The method of any one of statements 1-5, wherein the reporter construct comprises at least one nucleotide containing a non-natural sugar.
7. The method of any one of statements 1-6, wherein the label on the reporter construct is an enzyme selected from horse radish peroxidase and alkaline phosphatase.
8. The method of statement 7, wherein the reporter construct is conjugated to a magnetic bead.
9. The method of any one of statements 1-7, wherein the trigger nucleic acid sequence is a single-stranded DNA sequence or RNA.
10. The method of any one of statements 1-9, wherein either or both of the first target binding construct and second binding constructs is an antibody or antigen binding fragment thereof.
11. The method of any one of statements 1-10, wherein the first or second target binding construct is immobilised on a substrate or conjugated to a magnetic bead.
12. The method of statement 11, further comprising the step of performing magnetic separation of the captured target bound to the first target binding construct from the sample when the first target binding construct is conjugated to a magnetic bead.
13. The method of any one of statements 1-12, wherein the first target binding construct is an antibody or antigen binding fragment thereof conjugated to said type V or type VI CRISPR/Cas effector protein.
14. The method of any one of statements 1-12, wherein the first target binding construct is an antibody or antigen binding fragment thereof conjugated to said guide RNA.
15. The method of any one of statements 1-12, wherein the first target binding construct is an antibody or antigen binding fragment thereof conjugated to said type V or type VI CRISPR/Cas effector protein in combination with said guide RNA.
16. The method of any one of statements 1-12, wherein the first target binding construct is an antibody or antigen binding fragment thereof conjugated to said trigger nucleic acid sequence.
17. The method of any one of statements 2-16, wherein the first and second target binding constructs bind the same antigen.
18. The method of any one of statements 2-16, wherein the first and second target binding constructs bind different antigens or epitopes of the same target.
19. The method of any one of statements 1-18, wherein the target is selected from a cell, cellular component or cell surface marker, a small molecule, a peptide, a polypeptide, and a cytokine.
20. The method of statement 19, wherein the target is a cytokine.
21. The method of statement 20, wherein the cytokine is IFN-gamma.
22. The method of any one of statements 1-18, wherein the target is a whole cell or whole microorganism.
23. The method of any one of statements 1-18, wherein said target is a Giardia cyst or Cryptosporidium oocyst.
24. The method of statement 23, wherein said target is a Cryptosporidium oocyst.
25. The method of any one of statements 22-24, wherein the first target binding is an anti-cryptosporidium antibody.
26. The method of any one of statements 19, or 22-25, wherein the target is detected at single cell or single organism sensitivity.
27. The method of any one of statements 1-21, wherein the target is detected at femtomolar sensitivity or lower.
28. The method of any one of statements 1-27, wherein the sample is a biological sample or an environmental sample.
29. The method of statement 28, wherein the biological sample is a blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate, or fluid obtained from a joint, or a swab of skin or mucosal membrane surface, a tissue biopsy, a culture of cells or medium from cell culture.
30. The method of statement 29, wherein the sample is blood, plasma, serum or a biopsy obtained from a human patient.
31. The method of statement 28, wherein the sample is a water sample.
32. The method of statement 28, wherein the sample is a crude sample.
33. The method of any of statements 1-31, wherein the sample is a concentrated or purified sample.
34. A method for the detection of a target in a sample, the method comprising:
   (a) contacting the sample with:
      (i) a first target binding construct to thereby immobilise or capture the target;
      (ii) a second target binding construct;
      (iii) a third binding construct which binds to the second target binding construct;
      (iii) a type V or type VI CRISPR/Cas effector protein;
      (iv) a trigger nucleic acid sequence;
      (v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
      (vi) a labelled reporter construct, wherein said reporter construct comprises a nucleic acid that does not hybridize with the guide sequence of the guide RNA and is cleavable by the nuclease activity of the activated type V or type VI CRISPR/Cas effector protein; and
   (b) measuring a detectable signal produced following cleavage of the labelled nucleic acid reporter by the type V or type VI CRISPR/Cas effector protein, thereby detecting the immobilised or captured target;
   wherein the type V or type VI CRISPR/Cas effector protein, the trigger nucleic acid sequence, the guide RNA, the type V or type VI CRISPR/Cas effector protein in combination with the guide RNA, or the type V or type VI CRISPR/Cas effector protein in combination with the guide RNA and the trigger nucleic acid is linked or conjugated to the third binding construct to thereby co-locate the type V or type VI CRISPR/Cas effector protein and the target when present in the sample; and wherein the target is not a nucleic acid sequence.
35. The method of statement 34, wherein the type V or type VI CRISPR/Cas effector protein is a Cas12a (Cpf1) or Cas12b (C2c1) protein, or Cas 13a or Cas 13b.
36. The method of statement 34 or 35, wherein the reporter construct is a labelled RNA.
37. The method of any one of statements 34-36, wherein the trigger nucleic acid sequence is a DNA sequence.
38. The method of any one of statements 34-37, wherein the first target binding construct is an antibody or antigen binding fragment thereof.
39. The method of any one of statements 34-38, wherein the first target binding construct is immobilised on a substrate or conjugated to a magnetic bead.
40. The method of statement 39, further comprising the step of performing magnetic separation of the captured target bound to the first target binding construct from the sample when the first target binding construct is conjugated to a magnetic bead.
41. The method of any one of statements 34-40, wherein the second target binding construct is an antibody or antigen binding fragment thereof.
42. The method of statement 41, wherein the antibody or antigen binding fragment thereof is labelled.
43. The method of any one of statements 34-42, wherein the third binding construct is an antibody or antigen binding fragment thereof conjugated to said type V or type VI CRISPR/Cas effector protein.
44. The method of any one of statements 34-42, wherein the third binding construct is an antibody or antigen binding fragment thereof conjugated to said guide RNA.
45. The method of any one of statements 34-42, wherein the third binding construct is an antibody or antigen binding fragment thereof conjugated to said type V or type VI CRISPR/Cas effector protein and said guide RNA.
46. The method of any one of statements 34-42, wherein the third binding construct is an antibody or antigen binding fragment thereof conjugated to said trigger nucleic acid sequence.
47. The method of any one of statements 34-46, wherein the first and second target binding constructs bind the same antigen.
48. The method of any one of statements 34-46, wherein the first and second target binding constructs bind different antigens or epitopes of the same target.
49. The method of any one of statements 34-48, wherein the target is selected from a cell, cellular component or cell surface marker, a small molecule, a peptide, a polypeptide, and a cytokine.
50. The method of statement 49, wherein the target is a cytokine.
51. The method of statement 50, wherein the cytokine is IFN-gamma.
52. The method of any one of statements 34-48, wherein the target is a whole cell or whole microorganism.
53. The method of any one of statements 34-48, wherein said target is a Giardia cyst or Cryptosporidium oocyst.
54. The method of statement 53, wherein said target is a Cryptosporidium oocyst.
55. The method of any one of statements 34-54, wherein the first target binding is an anti-cryptosporidium antibody.
56. The method of any one of statements 52-55, wherein the target is detected at single cell or single organism sensitivity.
57. The method of any one of statements 34-51, wherein the target is detected at femtomolar sensitivity or lower.
58. The method of any one of statements 30-53, wherein the sample is a biological sample or an environmental sample.
59. The method of statement 58, wherein the biological sample is a blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate, or fluid obtained from a joint, or a swab of skin or mucosal membrane surface, a tissue biopsy, a culture of cells or medium from cell culture.
60. The method of statement 59, wherein the sample is blood, plasma, serum or a biopsy obtained from a human patient.
61. The method of statement 58, wherein the sample is a water sample.
62. The method of statement 58, wherein the sample is a crude sample.
63. The method of any of statements 34-61, wherein the sample is a concentrated or purified sample.
64. A kit for detecting a target in a sample, the kit comprising:
   (i) a first binding construct;
   (ii) a type V or type VI CRISPR/Cas effector protein;
   (iii) a trigger nucleic acid sequence;
   (iv) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
   (v) a labelled reporter construct, wherein said reporter construct comprises a nucleic acid that is single stranded, does not hybridize with the guide sequence of the guide RNA and is cleavable by the nuclease activity of the activated type V or type VI CRISPR/Cas effector protein;
   wherein the first binding construct binds to the target or to another agent which binds or attaches to the target, and wherein the type V or type VI CRISPR/Cas effector protein, the trigger nucleic acid sequence, the guide RNA, or the type V or type VI CRISPR/Cas effector protein in combination with the guide RNA, or the type V or type VI CRISPR/Cas effector protein in combination with the guide RNA and the trigger nucleic acid is linked or conjugated to the first binding construct to thereby co-locate the type V or type VI CRISPR/Cas effector protein and the target when present in the sample; and wherein the target is not a nucleic acid sequence.
65. A kit for detecting a target in a sample, the kit comprising:
   (i) a target binding construct;
   (ii) a second target binding construct;
   (iii) a type V or type VI CRISPR/Cas effector protein;
   (iv) a trigger nucleic acid sequence;
   (v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
   (vi) a labelled reporter construct, wherein said reporter construct comprises a nucleic acid that is single stranded, does not hybridize with the guide sequence of the guide RNA and is cleavable by the nuclease activity of the activated type V or type VI CRISPR/Cas effector protein;
   wherein the type V or type VI CRISPR/Cas effector protein, the trigger nucleic acid sequence, the guide RNA, or the type V or type VI CRISPR/Cas effector protein in combination with the guide RNA, or the type V or type VI CRISPR/Cas effector protein in combination with the guide RNA and the trigger nucleic acid is linked or conjugated to the second target binding construct so as to co-locate the type V or type VI CRISPR/Cas effector protein and the target when present in the sample; and wherein the target is not a nucleic acid sequence.
66. The kit of statement 60 or 61, wherein the first target binding construct is immobilised on a substrate or conjugated to a magnetic bead.
67. The kit of any one of statements 60-62, further comprising one or more of: a reaction buffer, a washing buffer, and reagents for recovering or releasing immobilised or captured target.
68. The method according to statement 23 or 53, further comprising step of recovering Giardia cysts or Cryptosporidium oocysts by releasing captured cysts or oocysts from the first and/or second target binding agent, when a detectable signal is measured.
69. The method of statement 68, further comprising detecting the number of cysts or oocysts present in the sample by microscopy.
70. The method of any one of statements 1-63, 68 or 69, wherein the sample is also contacted with at least one sulfhydryl reductant.
71. The method of statement 70, wherein the sulfhydryl reductant is selected from the group consisting of Dithiothreitol (DTT), Tris(2-carboxyethyl) phosphine (TCEP) to and 2-Mercaptoethanol (2-ME)).
72. The method of statement 71, wherein the sulfhydryl reductant is DTT.
73. The method of statement 72, wherein said contacting occurs at a temperature of about 37° C.
74. The method of any one of statements 1-63, or 68-73, wherein the sample is also contacted with at least one non-ionic surfactant.
75. The method of statement 74, wherein the non-ionic surfactant is selected from the group consisting of Brij L23 and poly(vinyl alcohol) (PVA), 76. The method of statement 75, wherein the non-ionic surfactant is PVA.
77. The method of any one of statements 1-63, or 68-76, wherein said contacting occurs in a reaction mixture comprising a buffer.
78. A method of enhancing a type V or Type VI CRISPR/Cas detection system comprising adding a sulfhydryl reductant, and/or a non-ionic surfactant to a reaction mixture comprising the type V or Type VI CRISPR/Cas effector of the system.
79. The method of statement 78, comprising adding a sulfhydryl reductant and a non-ionic surfactant to a reaction mixture comprising the type V or Type VI CRISPR/Cas effector of the system.
80. The method of statement 78 or 79, wherein the sulfhydryl reductant is selected from the group consisting of Dithiothreitol (DTT), Tris(2-carboxyethyl) phosphine (TCEP) to and 2-Mercaptoethanol (2-ME)).
81. The method of statement 80, wherein the sulfhydryl reductant is DTT.
82. The method of statement 78 or 79, wherein the non-ionic surfactant is selected from the group consisting of Brij L23 and poly(vinyl alcohol) (PVA).
83. The method of statement 82, wherein the non-ionic surfactant is PVA.
84. The method of statement 79, wherein the wherein the sulfhydryl reductant is DTT and the non-ionic surfactant is PVA.
85. The kit of any one of statements 64-67, wherein said a reaction buffer, washing buffer, and reagents for recovering or releasing immobilised or captured target comprise a sulfhydryl reductant, and/or a non-ionic surfactant.
86. The kit of statement 85, wherein the sulfhydryl reductant is selected from the group consisting of Dithiothreitol (DTT), Tris(2-carboxyethyl) phosphine (TCEP) to and 2-Mercaptoethanol (2-ME)); and wherein the non-ionic surfactant is selected from the group consisting of Brij L23 and poly(vinyl alcohol) (PVA).
87. The kit of statement 86, wherein the sulfhydryl reductant is DTT and the non-ionic surfactant is PVA.
88. The kit of any one of statements 64-67, or 86-88, when used for a method for detecting a target in a sample.
89. A reaction mixture comprising: (i) at least one target binding construct; (ii) a type V or type VI CRISPR/Cas effector protein; (iii) a trigger nucleic acid sequence; (v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and (iv) a labelled reporter construct, wherein said reporter construct comprises a nucleic acid that does not hybridize with the guide sequence of the guide RNA and is cleavable by the nuclease activity of the activated type V or type VI CRISPR/Cas effector protein; wherein the type V or type VI CRISPR/Cas effector protein, the trigger nucleic acid, the guide RNA, or the type V or type VI CRISPR/Cas effector protein in combination with the guide RNA optionally in further combination with the trigger nucleic acid, is conjugated to said at least first target binding construct.
90. The reaction mixture of statement 89, further comprising a sample.
91. The reaction mixture of statement 89 or 90, further comprising one or more of: a reaction buffer.
92. The reaction mixture of statement 91, further comprising a sulfhydryl reductant, and/or a non-ionic surfactant.
93. The reaction mixture of statement 92, wherein the sulfhydryl reductant is selected from the group consisting of Dithiothreitol (DTT), Tris(2-carboxyethyl) phosphine (TCEP) to and 2-Mercaptoethanol (2-ME)), and wherein the non-ionic surfactant is selected from the group consisting of Brij L23 and poly(vinyl alcohol) (PVA).
94. The reaction mixture of statement 93, wherein the sulfhydryl reductant is DTT, and wherein the non-ionic surfactant is PVA.

Any example or embodiment herein shall be taken to apply mutatis mutandis to any other example or embodiment unless specifically stated otherwise.

The present disclosure is not to be limited in scope by the specific examples described herein, which are intended for the purpose of exemplification only. Functionally equivalent methods and systems are clearly within the scope of the disclosure, as described herein.

The disclosure is hereinafter described by way of the following non-limiting Examples and with reference to the accompanying drawings.

### BRIEF DESCRIPTION OF DRAWINGS

FIG. 1 shows schematics for the CRISPR/Cas12a-involved whole cell biosensing system for detection. A simple 3 step procedure has been applied to realise the Cryptosporidium whole cell detection, using a similar setup as a common ELISA: (A) Step 1: Adding sample for target Cryptosporidium cell capture; (B) Step 2: Adding pre-made anti-Cryptosporidium Abs-ssDNA conjugate to form the antibody-cell sandwich structure; (C) Step 3: Adding the prepared CRISPR/Cas12a reaction mixture to recognize the triggering ssDNA on the Ab-ssDNA conjugate for Cas12a RNP activation. (D) Finally, collateral cleavage of activated CRISPR/Cas12a RNPs cuts the fluorescent quenched reporters to generate amplified signal for detection.

FIG. 2 shows Cryptosporidium imaged by confocal microscopy and antibody affinity test. Texas Red-labelled Cryptosporidium oocysts at (A) 60× magnification appear round with diameter of 4 um; (B) 40× magnification, cell concentration is 1600 oocysts/mL; (C) 20× magnification, cell concentration is 1600 oocysts/mL.

FIG. 3 shows demonstration of the antibody-cell sandwich structure. (A) Verifying the anti-Cryptosporidium antibody affinity. (1) Single Cryptosporidium oocysts with fluorescent signal generated from Texas Red fluorophores on its surface under 20× magnification; (2) The same oocyst shows fluorescent signal generated from the anti-Cryptosporidium antibody with FITC labelling; (3) Merged figures (1) and (2) show colocalisation of fluorescence signals in the examined Cryptosporidium oocyst; (B) Verification of the sensing interface for Cryptosporidium oocyst capture. Positive linear correlation between fluorescence intensity and Cryptosporidium oocyst concentration indicates a successful capture of Cryptosporidium oocysts on the sensing interface in proportion to the observed fluorescent signal (n=3); (C) Formation of the antibody-cell sandwich structure. After the Cryptosporidium oocyst has been captured onto the sensing interface, the increased fluorescence intensity of FITC-labelled antibody indicates the successful formation of the antibody-cell sandwich (n=3).

FIG. 4 shows verification of the anti-Cryptosporidium antibody affinity. (A) Negative control (no Cryptosporidium); (B) 500 oocysts/mL Cryptosporidium concentration; (C) 1000 oocysts/mL Cryptosporidium concentration. The parameters of confocal microscopy were set at 3% laser intensity, 500V PMT, 3% off-set, pin hole 1.00. Ex=488 nm and Em=500-540 nm. 20× magnification.

FIG. 5 shows optimization of the streptavidin for sensing interface on high binding 96-well plate. The fluorescence signal was detected using the iD3 plate reader, at Ex=490 nm, and Em=530 nm. (n=3).

FIG. 6 shows formation of the sensing interface with biotinylated anti-Cryptosporidium antibody as the capture antibody (number of replicates, n=3).

FIG. 7 shows distribution of anti-Cryptosporidium antigen on the Cryptosporidium oocyst surface. The parameters of confocal microscopy were set at 3% laser intensity, 500V PMT, 3% off-set, pin hole 1.00. Ex=488 nm and Em=500-540 nm. 60× magnification.

FIG. 8 shows preparation of anti-Cryptosporidium Abs-ssDNA conjugate for CRISPR/Cas12a-based signal amplification. (A) The schematics of the Abs-ssDNA conjugate synthesis; (B) Evidence that CRISPR/Cas12a reaction is triggered by Abs-ssDNA conjugate leading to signal amplification (n=3); (C) Verifying the uncompromised Cryptosporidium binding affinity for the synthesized Abs-ssDNA conjugate (n=3).

FIG. 9 shows verifying the formation of the Abs-ssDNA. After centrifugation-based filtration by using a low binding PES membrane (10 kD), the excess unbound ssDNA oligonucleotides were separated and removed due to their significantly lower molecular weight compared to the antibody. As here the fluorescence signal only came from the 3′ labelled Texas Red fluorophore, the fluorescence signal changes between the remaining and filtered samples represents the binding of ssDNA to the antibody. With the increase of the antibody concentration, the increased fluorescence signal (3′ labelled Texas Red fluorophore from the attached ssDNA) of the expected Abs-ssDNA conjugate indicated the successful formation of the Abs-ssDNA conjugates (n=6).

FIG. 10 shows Abs-ssDNA activation of Cas12a shows linear correlation with the amount of conjugate. (A) Results for 30 minutes CRISPR/Cas12a reaction (n=3); (B) Results for 60 minutes CRISPR/Cas12a reaction. 30 minutes reaction is enough for generating a satisfactory linear correlation (n=3). The standard CRISPR/Cas12a reaction mixture in this study was prepared as described at Method S4. Then, 5 μL of pre-made Abs-ssDNA conjugate with different concentrations (0, 1.25, 2.5, 5, 10 μg/mL) was mixed with 100 μL of prepared CRISPR/Cas12a reaction mixture. The reaction was set at room temperature and the fluorescence signal was detected by plate reader (iD3, Molecular Devices), with Ex=570 nm and Em=615 nm.

FIG. 11 shows CRISPR/Cas12a-based whole cell immunoassay for Cryptosporidium detection. (A) Assay response across a wide range of Cryptosporidium oocyst concentrations and linear fit (n=4). After 30 mins of the CRISPR/Cas12a reaction, the linear range was found to be from 6.25 to 1600 oocysts/mL. As 100 μL sample has been used for the test, the lowest concentration of 6.25 oocysts/mL is approximately equal to 0-1 Cryptosporidium oocyst per sample (n=4, so 0 in some replicates and 1 in other replicates), which explained insignificant fluorescence intensity differences between 0 and 6.25 oocysts/mL seen in the insert; (B) System sensitivity test for samples with precise Cryptosporidium oocyst numbers (n=3); (C) System selectivity test in 4 different complex sample matrixes with spiked Cryptosporidium (800 oocysts/mL, n=3).

FIG. 12 shows Cryptosporidium oocyst detection in samples with precisely dispensed numbers of oocysts at different CRISPR/Cas12a reaction times. The fluorescent signal was then continuously monitored by plate reader (iD3, Molecular Devices) with Ex=570 nm and Em=615 nm. (n=3).

FIG. 13 shows comparison of CRISPR/Cas12a amplification efficiency to Texas Red labelled antibody. Ex=570 nm and Em=615 nm. Data indicated that, without the CRISPR/Cas12a-based signal amplification, the detection sensitivity is 160 oocysts per sample.

FIG. 14 shows controls for working with different complex sample matrixes. 4 different types of samples (yogurt, orange juice, lite milk, dirt suspension) were firstly diluted 10 times by 1×PBS buffer. The fluorescent signals were then detected by plate reader (iD3, Molecular Devices) with Ex=570 nm and Em=615 nm. (n=3).

FIG. 15 shows Cryptosporidium detection in mud samples from water treatment plant. (A) System background signal changes in mud samples with different dilution times (n=3); (B) Comparison of system performance between PBS sample and mud sample. 5 Cryptosporidium oocysts have been spiked into each sample (n=3); (C) Basic steps for Cryptosporidium detection in the mud sample, with the same ELISA-like protocol, but an additional sample dilution procedure; (D) Cryptosporidium detection in mud samples with ultra-low oocyst concentrations of individually dispensed oocysts (n=3).

FIG. 16 shows detection of precisely dispensed Cryptosporidium oocysts in mud samples at different reaction times. The fluorescent signal was then tested by plate reader (iD3, Molecular Devices) with Ex=570 nm and Em=615 nm. (n=3) The plate then has been incubated overnight at room temperature at Dark, and the fluorescent signal was tested again. Blue=0 cells, orange=1 oocyst per sample, grey=5 oocysts per sample, yellow=10 oocysts per sample.

FIG. 17 shows the optimization of gRNA and RNA reporter in the CRISPR/Cas12a system. (A) The optimization of gRNA molar ratio in the CRISPR/Cas12a system; (B) the optimization of RNA reporter ratio in the CRISPR/Cas12a system. The number in Figure B represents the molar ratio of RNA reporter to Cas12a effector.

FIG. 18 shows optimization of RNA reporter. (A) Optimization of RNA reporter based CRISPR/Cas12a system using diverse chemicals; (B) comparison of modified RNA reporter with conventional DNA reporter in CRISPR/Cas12a system. RNA reporter-modified represents DTT, 37 in figure A.

FIG. 19 shows application of optimized RNA reporter based CRISPR/Cas12a system for the detection of Crypto.

FIG. 19a shows (A) The schematic for synthesis the BAC protein conjugate; (B) Investigation of the formation of BAC conjugate using Zeta-sizer. Plot shows size distributions of Ab, Cas/gRNA and BAC (C) Verification of BAC conjugate formation using EMSA. Lines: 1=100 bp DNA Ladder; 2=BAC conjugate; 3=Cas/gRNA RNP; 4=Antibody (with ssDNA oligo conjugated; 5=100 bp DNA Ladder.

FIG. 20 shows evaluation of the trans-cleavage ability of antibody-Cas12a/gRNA conjugate. (A) Investigation of the concentration dependent trans-cleavage ability of Cry104-Cas12a/gRNA conjugate; (B) Evaluation of the trans-cleavage ability of pure Cas12a, antibody-Cas12a conjugate, and antibody-Cas12a/gRNA (BAC) conjugate (P<0.05, P<0.001); (C) Enhancement of the trans-cleavage ability of antibody-Cas12a/gRNA conjugate; (D) Investigation of the stability of antibody-Cas12a/gRNA conjugate. (E) Comparison of the trans-cleavage efficiency of Cas12a RNPs under different status. Cas=unconjugated Cas12a RNP, Abs-Cas=antibody conjugated Cas12a, Abs-Cas/gRNA=antibody conjugated Cas12a with gRNA loaded before conjugation. (F) Control test for Cas12a induced trans-cleavage activity. Neg−=unconjugated antibody without Cas12a. Abs-Cas/gRNA=antibody conjugated Cas12a with gRNA loaded before conjugation.

FIG. 20a shows evaluation BAC conjugate induced trans-cleavage activity. The same concentration of triggering ssDNA (5 μL 1 μM) has been used to trigger the trans-cleavage activity of 100 μL different concentrations of prepared BAC, including 1.05 μg/mL, 2.1 μg/mL, 4.2 μg/mL, 8.4 μg/mL, within 1×NEB 2.1 buffer with 166 nM ssDNA labelled with Texas Red.

FIG. 20b shows the reaction time of Cas12a-based trans-cleavage activity for fluorescent signal generation. Control=BAC conjugate in original reaction buffer, DTT=BAC conjugate in reaction buffer with 10 mM DTT, DTT&37° C.=BAC conjugate in reaction buffer with 10 mM DTT and under heated temperature to 37° C. The use of DTT or the combination of DTT and heated temperature will significantly boost the trans-cleavage activity, hence, reduce the reaction time for reaching desired fluorescent signal intensity. Here, ˜50% faster with DTT, and ˜70% faster with DTT under 37° C. reaction temperature.

FIG. 21 shows evaluation of the recognition ability of Cry104-Cas12a/gRNA conjugate. (A) Apply the Cry104-Cas12a/gRNA conjugate for the recognition of Tex-Crypto using plate based method; (B) comparison of the recognition ability of Cry104-Cas12a/gRNA conjugate with pristine Cry104.

FIG. 22 shows application of Cry104-Cas12a/gRNA based CRISPR/Cas12a system for the detection of Crypto.

FIG. 23 shows optimization of the concentrations of sulfhydryl reductants in CRISPR/Cas12a biosensing system (Texas red based DNA reporter). (A) Optimization of DTT concentration; (B) Optimization of TCEP concentration; (C) Optimization of 2-ME concentration.

FIG. 24 shows sulfhydryl reductant-induced trans-cleavage enhancement for CRISPR/Cas12a. (A) Application of sulfhydryl reductant in CRISPR/Cas12a reaction buffer system; (B) Demonstration of the enhancement effect of activator DTT to CRISPR/Cas12a system using electrophoretic mobility shift assay; (C) Demonstration of the enhancement effect of activator TCEP to CRISPR/Cas12a system using electrophoretic mobility shift assay. (The gel figures from left to right: 1. 10 bp ladder; 2. No DTT/TCEP, 1 μL 10 μM trigger ssDNA 5-bio, 24 nt; 3. No DTT/TCEP, 1 μL 5 μM trigger ssDNA 5-bio, 24 nt; 4. No DTT/TCEP, 1 μL 2 μM trigger ssDNA 5-bio, 24 nt; 5. No DTT/TCEP, 1 μL 1 μM trigger ssDNA 5-bio, 24 nt; 6. 10 bp ladder; 7. DTT/TCEP, 1 μL 10 μM trigger ssDNA 5-bio, 24 nt; 8. DTT/TCEP, 1 μL 5 μM trigger ssDNA 5-bio, 24 nt; 9. DTT/TCEP, 1 μL 2 μM trigger ssDNA 5-bio, 24 nt; 10. DTT/TCEP, 1 μL 1 μM trigger ssDNA 5-bio, 24 nt; 11. 10 bp ladder 12. No DTT, No trigger; 13. 10 bp ladder.

FIG. 25 shows (A) Evaluation of the enhancement effect of activator DTT to CRISPR/Cas12a system using electrophoretic mobility shift assay (From left to right: 1. 10 bp ladder; 2. Cas12a mix with 1.5 μL 10 μM reporter; 3. Cas12a mix (No DTT) with 1.5 uL 10 uM reporter+1 μL 1 μM trigger; 4. Cas12a mix (DTT) with 1.5 μL 10 μM reporter+1 μL 1 μM trigger; 5. Cas12a mix with 2 μL 10 μM reporter; 6. Cas12a mix (No DTT) with 2 μL 10 μM reporter+1 μL 1 μM trigger; 7. Cas12a mix (DTT) with 2 μL 10 μM reporter+1 μL 1 μM trigger; 8. 10 bp ladder); and (B) Evaluation of the enhancement effect of activator TCEP to CRISPR/Cas12a system using electrophoretic mobility shift assay (From left to right: 1. 10 bp ladder; 2. Cas12a mix with 1 μL 5 μM reporter; 3. Cas12a mix (No TCEP) with 1 μL 5 μM reporter+1 μL 1 μM trigger; 4. Cas12a mix (TCEP) with 1 μL 5 μM reporter+1 μL 1 μM trigger; 5. Cas12a mix with 1 μL 10 μM reporter; 6. Cas12a mix (No TCEP) with 1 μL 10 μM reporter+1 μL 1 μM trigger; 7. Cas12a mix (TCEP) with 1 μL 10 μM reporter+1 μL 1 μM trigger; 8. 10 bp ladder; 9. Cas12a mix with 3 μL 5 μM reporter; 10. Cas12a mix (No TCEP) with 3 μL 5 μM reporter+1 μL 1 μM trigger; 11. Cas12a mix (TCEP) with 3 μL 5 μM reporter+1 μL 1 μM trigger; 12. Cas12a mix with 2 μL 10 μM reporter; 13. Cas12a mix (No TCEP) with 2 μL 10 μM reporter+1 μL 1 μM trigger; 14. Cas12a mix (TCEP) with 2 μL 10 μM reporter+1 μL 1 μM trigger; 15. 10 bp ladder).

FIG. 26 shows the optimization of non-ionic surfactant concentrations in CRISPR/Cas12a biosensing system (Texas red conjugated DNA reporter). (A) Brij L23; (B) Tween 20; (C) Triton X-100; (D) PEG; (E) PVA.

FIG. 27 shows non-ionic surfactant induced trans-cleavage enhancement for CRISPR/Cas12a biosensing system. (A) Application of non-ionic surfactant in CRISPR/Cas12a biosensing system; (B) Demonstration of the enhancement effect of activator Brij L23 to CRISPR/Cas12a system using electrophoretic mobility shift assay; (C) Demonstration of the enhancement effect of activator PVA to CRISPR/Cas12a system using electrophoretic mobility shift assay. (The gel figures from left to right: 1. 10 bp ladder; 2. No Brij L23/PVA, 1 μL 10 μM trigger ssDNA 5-bio, 24 nt; 3. No Brij L23/PVA, 1 μL 5 μM trigger ssDNA 5-bio, 24 nt; 4. No Brij L23/PVA, 1 μL 2 μM trigger ssDNA 5-bio, 24 nt; 5. No Brij L23/PVA, 1 μL 1 μM trigger ssDNA 5-bio, 24 nt; 6. No Brij L23/PVA, no trigger; 7. 10 bp ladder; 8. Brij L23/PVA, 1 μL 10 μM trigger ssDNA 5-bio, 24 nt; 9. Brij L23/PVA, 1 μL 5 μM trigger ssDNA 5-bio, 24 nt; 10. Brij L23/PVA, 1 μL 2 μM trigger ssDNA 5-bio, 24 nt; 11. Brij L23/PVA, 1 μL 1 μM trigger ssDNA 5-bio, 24 nt; 12. Brij L23/PVA, no trigger; 13. 10 bp ladder).

FIG. 28 shows (A) Evaluation of the enhancement effect of activator Brij L23 to CRISPR/Cas12a system using electrophoretic mobility shift assay (From left to right: 1. 10 bp ladder; 2. Cas12a mix with 1 uL 5 uM reporter; 3. Cas12a mix (No 0.04% Tw20) with 1 μL 5 μM reporter+1 μL 1 μM trigger; 4. Cas12a mix (0.04% Tw20) with 1 μL 5 μM reporter+1 μL 1 μM trigger; 5. Cas12a mix with 2 μL 5 μM reporter; 6. Cas12a mix (No 0.04% Tw20) with 2 μL 5 μM reporter+1 μL 1 μM trigger; 7. Cas12a mix (0.04% Tw20) with 2 μL 5 μM reporter+1 μL 1 μM trigger; 8. 10 bp ladder; 9. Cas12a mix with 2 μL 10 μM reporter; 10. Cas12a mix (No 0.04% Tw20) with 2 μL 10 μM reporter+1 μL 1 μM trigger; 11. Cas12a mix (0.04% Tw20) with 2 μL 10 μM reporter+1 μL 1 μM trigger; 12. Cas12a mix with 3 μL 10 μM reporter; 13. Cas12a mix (No 0.04% Tw20) with 3 μL 10 μM reporter+1 μL 1 μM trigger; 14. Cas12a mix (0.04% Tw20) with 3 μL 10 μM reporter+1 μL 1 μM trigger; 15. 10 bp ladder); and (B) Evaluation of the enhancement effect of activator PVA to CRISPR/Cas12a system using electrophoretic mobility shift assay (From left to right: 1. 10 bp ladder; 2. Cas12a mix with 1 μL 5 μM reporter; 3. Cas12a mix (No PVA) with 1 μL 5 μM reporter+1 μL 1 μM trigger; 4. Cas12a mix (PVA) with 1 μL 5 μM reporter+1 μL 1 μM trigger; 5. Cas12a mix with 2 μL 5 μM reporter; 6. Cas12a mix (No PVA) with 2 μL 5 μM reporter+1 μL 1 μM trigger; 7. Cas12a mix (PVA) with 2 μL 5 μM reporter+1 μL 1 μM trigger; 8. 10 bp ladder; 9. Cas12a mix with 2 μL 10 uM reporter; 10. Cas12a mix (No PVA) with 2 μL 10 μM reporter+1 μL 1 μM trigger; 11. Cas12a mix (PVA) with 2 μL 10 μM reporter+1 μL 1 μM trigger; 12. Cas12a mix with 3 μL 10 μM reporter; 13. Cas12a mix (No PVA) with 3 μL 10 μM reporter+1 μL 1 μM trigger; 14. Cas12a mix (PVA) with 3 μL 10 μM reporter+1 μL 1 μM trigger; 15. 10 bp ladder).

FIG. 29 shows application of Ionic surfactant to CRISPR/Cas12a biosensing system (Texas red based DNA reporter). (A) Application of anionic surfactant NaDC to CRISPR/Cas12a biosensing system; (C) Application of anionic surfactant SDS to CRISPR/Cas12a biosensing system; (D) Application of cationic surfactant CTAB to CRISPR/Cas12a biosensing system.

FIG. 30 shows characterization of chemical enhanced trans-cleavage ability of Cas12a. (A) The comparison and combination of sulfhydryl reductant with non-ionic surfactants; (B) The specificity of fluorophore difference on the target ssDNA of Cas12a; (C) The specificity of RNA target of Cas12a.

FIG. 31 shows comparison of the difference of ssDNA and ssRNA reporter based CRISPR/Cas12 biosensing system. (A) ssDNA reporter based CRISPR/Cas12a biosensing system in room temperature (RT) (Texas red); (B) ssRNA reporter based CRISPR/Cas12a biosensing system in RT and 37° C. (FAM).

FIG. 32 shows application chemical enhanced CRISPR/Cas12a biosensing system for the detection of HP and SARS-CoV-2 gene. (A) Evaluation of the incubation time of optimized and original CRISPR/Cas12a biosensing system for the detection of HP gene; (B) Evaluation of the performance of optimized and original CRISPR/Cas12a biosensing system for the detection of HP gene; (C) Evaluation of the sensitivity of optimized and original CRISPR/Cas12a system for the detection of HP gene; (D) Evaluation of the incubation time of optimized and original CRISPR/Cas12a biosensing system for the detection of SARS-CoV-2 gene; (E) Evaluation of the performance of optimized and original CRISPR/Cas12a biosensing system for the detection of SARS-CoV-2 gene; (F) Evaluation of the sensitivity of optimized and original CRISPR/Cas12a system for the detection of SARS-CoV-2 gene.

FIG. 33 shows application of chemical activators to CRISPR/Cas13a biosensing system. (A) Optimization of DTT concentration to CRISPR/Cas13a biosensing system; (B) Optimization of PVA concentration to CRISPR/Cas13a biosensing system.

FIG. 34 shows chemically enhanced CRISPR/Cas13a biosensing system for the detection of SARS-CoV-2 gene. (A) Evaluation of the performances of enhancer DTT and PVA to CRISPR/Cas13a biosensing system; (B) Evaluation of the incubation time for the detection of SARS-CoV-2 gene using optimized and original CRISPR/Cas13a system; (C) Evaluation of the performance of optimized and original CRISPR/Cas13a system for the detection of SARS-CoV-2 gene; (D) Evaluation of the sensitivity of optimized and original CRISPR/Cas13a system for the detection of SARS-CoV-2 gene.

FIG. 35 shows schematics of CRISPR-based Universal Immunoassay Signal Enhancer (CRUISE). platform. The key elements for CRUISE to bridge the CRISPR/Cas12a and immunoassays is the Abs-ssDNA conjugate, which has 3 components, including the selected antibody, streptavidin, and biotinylated triggering ssDNA. The prepared Abs-ssDNA conjugate can be used either as primary antibody or secondary antibody in different immunoassay schemes. For the primary antibody-based approach, (A) Abs-ssDNA can directly recognized the target analyte as the single antibody in the system or (B) as the detection antibody on a typical antibody-analyte sandwich structure; (C) for the secondary antibody-based approach, instead of targeting the analyte, the Abs-ssDNA targeting the Fc region of another antibody. Both these two CRUISE approaches share a similar three-step procedure: 1. Recognition of the designated target by the Abs-ssDNA; 2. Addition of CRISPR/Cas12a reaction mixture, which includes the CRISPR/Cas12a RNP able to recognize the triggering ssDNA and fluorescent quenched reporters; 3. Activation of CRISPR/Cas12a RNPs for random cutting the surrounding reporters to generate amplified fluorescent signal. This fluorescent signal intensity is detected to reveal the concentration of target analyte present in the sample.

FIG. 36 shows (A) Verification of Abs-ssDNA synthesis by centrifugation-based filtration. Increased level of Texas Red labelled triggering ssDNA indicates the remaining large molecule weight component, here the successfully bound Abs-ssDNA conjugate (number of replicates, n=3); (B) Demonstration of the quantitative binding of triggering ssDNA to antibody (n=3); (C) Demonstration of the of antibody affinity in Abs-ssDNA conjugate (n=3); (D) Universality of activating CRISPR/Cas12a RNP through various Abs-ssDNA conjugates (n=3).

FIG. 37 shows activation of CRISPR/Cas12a reaction system by designed triggering ssDNA (n=3).

FIG. 38 shows reproducibility test for Abs-ssDNA triggered CRISPR/Cas12a trans-cleavage activity (n=3).

FIG. 39 shows activation of CRISPR/Cas12a RNPs by different length of triggering ssDNA (n=3).

FIG. 40 shows optimization of conjugation ratio between antibody and triggering ssDNA (n=3), Abs=antibody, ssDNA=triggering ssDNA.

FIG. 41 shows controls for verifying different components in the CRUISE system (n=3). C-antibody=Capture antibody.

FIG. 42 shows optimization of Abs-ssDNA conjugate concentration (n=3).

FIG. 43 shows streptavidin facilitated capture antibody fixation (n=3).

FIG. 44 shows optimization of streptavidin for sensing interface establishment (n=3).

FIG. 45 shows optimization of capture antibody concentration in CRUISE (n=3).

FIG. 46 shows optimization of the ratio between Cas12a protein and gRNA for the CRISPR/Cas12a reaction mixture (n=3).

FIG. 47 shows optimization of concentration for CRISPR/Cas12a RNPs (n=3).

FIG. 48 shows the stability of implemented CRISPR/Cas12a amplification module for fluorescence signal amplification (n=3), F=positive signal after CRISPR/Cas12a activation, F0=inactivated CRISPR/Cas12a reaction mixture.

FIG. 49 shows the effect of reaction temperature on the CRISPR/Cas12a reaction (n=3).

FIG. 50 shows primary antibody derived CRUISE. (A) Demonstration of quantitative correlations between CRUISE and concentration of target analyte (n=3); (B) Specificity test of CRUISE for IFN-γ detection (n=3); (C) Linear range of CRUISE for IFN-γ detection. The range is 1 fg mL⁻¹ to 1 ng mL⁻¹ with the correlation coefficient R²=0.9419. The insert indicates that the sensitivity is 1 fg mL⁻¹ (0 to 1 fg mL⁻¹ of IFN-γ: p<0.05, two tailed t-test), and the calculated Limit of Detection (LOD)=5.65 fg mL⁻¹ (n=3); (D) Linear range of CRUISE for EGFR detection. The range is 1 fg mL⁻¹ to 1 ng mL⁻¹ with the correlation coefficient R²=0.9335. The insert indicated that the sensitivity is 1 fg mL⁻¹ (0 to 1 fg mL⁻¹ of EGFR: p<0.05, two tailed t-test), and the calculated LOD=5.05 fg mL⁻¹ (n=3); (E) Linear range of CRUISE for IFN-γ detection within plasma diluent samples. The range is 1 fg mL⁻¹ to 1 ng mL⁻¹ with the correlation coefficient R²=0.9886. The insert indicates that the sensitivity is 1 fg mL⁻¹ (0 to 1 fg mL⁻¹ of IFN-γ: p<0.05, two tailed t-test), and the calculated LOD=4.56 fg mL⁻¹ (n=3).

FIG. 51 Reproducibility test for the CRUISE system (n=3).

FIG. 52 Commercial ELISA kit for IFN-γ detection (n=3).

FIG. 53 shows CRUISE for non-sandwich immunoassay. The range is 200 fg mL⁻¹ to 2 ng mL⁻¹ with the correlation coefficient R²=0.9021. The practical sensitivity is 200 fg mL-1 (p<0.05, two tailed t-test) (n=3).

FIG. 54 shows secondary antibody derived CRUISE (iCRUISE). (A) Verification of anti-IgG Abs-ssDNA conjugate for CRISPR/Cas12a activation (n=3); (B) Verifying the target binding capability of the prepared anti-IgG Abs-ssDNA conjugate (n=3); (C) Demonstration of the quantitative correlation between prepared anti-IgG Abs-ssDNA conjugate and its target antibody (n=3); (D) Linear range of iCRUISE for IFN-γ detection. The range is 1 fg mL⁻¹ to 1 ng mL⁻¹ with the correlation coefficient R²=0.9624. The insert indicated that the sensitivity is 1 fg mL⁻¹ (0 to 1 fg mL⁻¹ of IFN-γ: p<0.05, two tailed t-test), and the calculated LOD=6.95 fg mL⁻¹ (n=3); (E) Linear range of iCRUISE for EGFR detection. The range is 1 fg mL⁻¹ to 1 ng mL⁻¹ with the correlation coefficient R²=0.9739. The insert indicated that the sensitivity is 1 fg mL⁻¹ (p<0.05, two tailed t-test), and the calculated Limit of Detection LOD=9.48 (n=3).

FIG. 55 shows schematics of the CRISPR/Cas12a-based ELISA Sensitivity Amplifier.

FIG. 56 shows CRISPR/Cas12a activation efficiency for modified triggering ssDNA. Different types of terminal modified triggering ssDNA have no significant difference in CRISPR/Cas12a activation efficiency (n=3). Experiment was done using Method S1.

FIG. 57 shows CRISPR/Cas12a activation status for different concentrations of triggering ssDNA (pM). Higher concentration of triggering ssDNA led to higher CRISPR/Cas12a activation level, and the final signal intensity can continuously increase with reaction time increased (n=3). Experiment was done using Method S2.

FIG. 58 shows CRISPR/Cas12a activation and the ssDNA-Abs conjugate synthesis; (A) CRISPR/Cas12a activation by free triggering ssDNA. Positive correlation is observed between free triggering ssDNA concentrations and the fluorescence intensity (n=3); (B) Demonstration of the formation of the anti-HRP ssDNA-Abs conjugate through filtration-based centrifugation (n=3). The fluorescence signal was generated by the Texas Red labelled triggering ssDNA; (C) Verification of the formed ssDNA-Abs conjugate by electrophoretic mobility shift assay (EMSA). By forming the dsDNA with the complementary strand of triggering ssDNA, the appearance of the 30 bp band verified that the triggering ssDNA is at its designated 30 nt length.

FIG. 59 shows anti-HRP ssDNA-Abs conjugate-induced CRISPR/Cas12a activation. (A) Demonstration of the CRISPR/Cas12a activation by anti-HRP ssDNA-Abs conjugate (n=3); (B) Concentration dependent CRISPR/Cas12a activation of the ssDNA-Abs conjugate (n=3); (C) Comparison of the activation efficiency between free triggering ssDNA and the formed anti-HRP ssDNA-Abs conjugates (n=3).

FIG. 60 shows CRISPR/Cas12a activation efficiency by different concentrations of anti-HRP ssDNA-Abs conjugates. Higher concentration of ssDNA-Abs conjugate can lead to higher CRISPR/Cas12a activation level, and the final intensity is also time dependent (n=3). Experiment was done using Method S3.

FIG. 61 shows CRISPR/Cas12a activation efficiency changes by using different antibody for ssDNA-Abs conjugation. The CRISPR/Cas12a activation efficiency for ssDNA-Abs conjugates with different antibodies shown no significant difference, and all can successfully trigger CRISPR/Cas12a collateral cleavage activity efficiently (n=3). Experiment was done using Method S4.

FIG. 62 shows Performance of the anti-HRP ssDNA-Abs conjugate in target recognition. (A) Verifying the target binding affinity of the unconjugated anti-HRP antibody (n=3); (B) Verifying the capture of the HRP-labelled detection antibody from ELISA kit by anti-HRP ssDNA-Abs conjugate (n=3). No Anti-HRP=No anti-HRP ssDNA-Abs conjugate, No D-Abs=No HRP-labelled detection antibody; (C) Demonstration the HRP binding affinity of anti-HRP ssDNA-Abs conjugation (n=3). F=fluorescence with HRP and anti-HRP ssDNA-Abs conjugate, F0=fluorescence with only anti-HRP ssDNA-Abs conjugate, EP=empty plate (background signal); (D) Optimization of the anti-HRP ssDNA-Abs conjugate concentration for the CES-Amplifier (n=3).

FIG. 63 shows Rabbit anti-HRP ssDNA-Abs conjugate for HRP-D-Abs recognition. Rabbit anti-HRP ssDNA-Abs conjugate shown no specific binding affinity to the HRP labelled detection antibody (HRP-D-Abs) from the commercial ELISA kit (Abcam, ab174443) (n=3). Experiment was done using Method S5.

FIG. 64 shows CRISPR/Cas12a activation efficiency by Anti-HRP ssDNA-Abs conjugate. (Anti-HRP ssDNA-Abs conjugate concentrations to detection antibody binding). After the immune-sandwich structure has been formed by the commercial ELISA kit, higher concentration of anti-HRP ssDNA-Abs conjugate can generate higher fluorescence signal for final detection (n=3). Experiment was done using Method S6.

FIG. 65 shows performance of the CES-Amplifier. (A) Effect of the antibody dilution buffer from the commercial ELISA kit on the CRISPR/Cas12a activity (n=3). Experiment was done using Method 5; (B) Effect of the wash buffer from the commercial ELISA kit on the CRISPR/Cas12a activity (n=3). Experiment was done using Method 6; (C) Linear range of CES-Amplifier for sensitivity enhancement of the commercial ELISA kit. Experiment was done using Method 8. The range is 1.2 pg/mL to 5 ng/mL (3 orders of magnitude) with the correlation coefficient R=0.9907 (n=6). The insert shows that the sensitivity is 1.2 pg/mL, as there is a significant difference between fluorescent signal intensities at 0 and 1.2 pg/mL analyte concentration (p<0.001 under two tailed t-test); (D) The corresponding analyte detection results by the original ELISA kit protocol, a significant difference between absorption intensities at 0 and 312.5 pg/mL analyte concentration indicates the sensitivity of the original commercial ELISA kit is 312.5 pg/mL (n=3).

FIG. 66 shows effects of commercial ELISA antibody dilution buffer to Abs-ssDNA conjugate and CRISPR/Cas12a activity. Experiment was done using Method S7. The Abs-ssDNA conjugate was firstly diluted in either ELISA kit Abs buffer (A) or PBS (C). In addition, two buffers with no ssDNA-Abs conjugate have been used as controls. Then, each of the treated Abs-ssDNA conjugate solution or controls has been used to trigger the CRISPR/Cas12a mixture. In the left figure, the results shown that ELISA Abs buffer treated Abs-ssDNA has much lower triggering efficiency (A) comparing to the PBS treated one (C). Even after overnight, right figure shown that the final signal intensity is still significantly different (P<0.05). Therefore, the Abs dilution buffer from ELISA kit is harmful to our CES-Amplifier system (n=3).

FIG. 67 shows colorimetric intensity changes for the additional wash step using 1×PBS. After the sandwich structure has been formed following the commercial ELISA kit instruction, before applying the anti-HRP ssDNA-Abs conjugate, the wash buffer for the additional wash step has been replaced by 1×PBS buffer. After applying the 1×PBS buffer for the additional washing step, the stability of the formed sandwich structure has also been tested by using the original TMB substrate of ELISA kit, and compared to the one without the additional wash. The results shown no significant absorption intensity change (n=3). Experiment was done using Method S8.

FIG. 68 shows synthesis and optimization of magnetic beads-ssDNA-HRP reporter. (A) a schematic of HPR reporter based CRISPR/Cas12a biosensing system and synthesis of magnetic beads-ssDNA-HRP reporter; (B) Optimization of ssDNA concentration; (C) Optimization of HRP concentration.

FIG. 69 shows evaluation and application of new HRP reporter. (A) Evaluation of new HRP reporter in OPD solution; (B) Optimization of HRP reporter concentration in CRISPR/Cas12a mixture; (C) Application of HRP reporter based CRISPR/Cas12a biosensing system for the detection of target nucleic acid.

FIG. 70 shows a schematic of Xeno Nucleic Acid (XNA) reporter based CRISPR/Cas12 biosensing system.

FIG. 71 shows evaluation the performances of XNA reporters in CRISPR/Cas12a biosensing system.

FIG. 72 shows application of BAC-based CRISPR/Cas12a system for the detection of cytokine IFN-γ. (A) the trans-cleavage activity of IFN-γ-based BAC conjugate; (B) Demonstration of the IFN-γ binding affinity in the IFN-γ-based BAC conjugate; (C) schematic for using IFN-γ-based BAC conjugation as detection antibody in a typical sandwich immunoassay for the detection of IFN-γ; (D) Quantitative detection of IFN-γ by using the BAC conjugate in the immunoassay. The detection limit reaches 10 fg/mL (P=0.004), and the linear range detection range is from 10 fg/mL to 10 pg/mL.

### DEFINITIONS

Throughout this specification, unless the context clearly requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term ‘consisting of’ means consisting only of.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (i.e., at least one) of the grammatical object of the article. By way of example, reference to ‘an element’ means one element, or more than one element.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

Reference throughout this specification to “one embodiment,” “an example embodiment,” means that a particular feature, structure, or characteristic described in connection with the embodiment is included in at least one embodiment of the present invention. Thus, the appearances of the phrases “in one embodiment,” “in an embodiment,” or “exemplary embodiment” in various places throughout this specification are not necessarily all referring to the same embodiment, but may. Furthermore, the particular features, structures, or characteristics may be combined in any suitable manner in one or more embodiments, as will be apparent to those of ordinary skill in the art from this disclosure. Furthermore, although some embodiments described herein include some but not other features included in other embodiments, combinations of features of different embodiments are intended to be within the scope of the invention. For example, in the appended claims, any of the claimed embodiments may be used in any combination.

In order that the present technology may be more clearly understood, preferred embodiments will be described with reference to the following examples. The present disclosure is not to be limited in scope by the specific examples described herein, which are intended for the purpose of exemplification only. Functionally-equivalent methods and systems are clearly within the scope of the disclosure, as described herein.

### DETAILED DESCRIPTION

### Overview

The capability of type V CRISPR/Cas proteins, e.g., Cas12 proteins such as Cpf1 (Cas12a) and C2c1 (Cas12b) to promiscuously cleave non-targeted single stranded DNA/RNA (ssDNA/ssRNA) once activated by detection of a target DNA (double or single stranded) has been reported previously. Similar capabilities have previously been reported for Type VI Cas effectors e.g. Cas13a, Cas13b Cas 13c etc., but their trans-cleavage is only effective on ssRNA. Once a type V CRISPR/Cas effector protein (e.g., a Cas12 protein such as Cas12a. Cas12b, Cas12c, Cas12d, Cas12e or Gas 13 protein such as Cas 13a, Cas13b Cas 13c) is activated by a guide RNA, resulting from hybridization of the guide RNA to a target sequence of a target DNA (e.g. a targeted DNA sequence in a sample), the protein becomes a nuclease that promiscuously cleaves nucleic acids (i.e. ssDNA, dsDNA or ssRNA for Type V effectors, or ssRNA for type V1 effectors) present (e.g. to which the guide sequence of the guide RNA does not hybridize). Thus, when the target DNA is present in the sample (e.g., in some cases above a threshold amount), the result is cleavage of nucleic acids, e.g. ssDNAs in the sample, which can be detected using any convenient detection method (e.g., using a labelled single stranded detector DNA).

As described above this capability has been exploited for use in methods of biosensing, primarily for pathogen detection based on nucleic acid sequences of the pathogens.

Provided herein are methods, kits and compositions which enable the detection of a non-nucleic acid target in a sample by utilizing the non-specific nuclease activity (i.e. cleavage of ssDNA, dsDNA, or ssRNA) of an activated type V or type VI CRISPR/Cas effector protein in combination with target binding constructs to facilitate ultrasensitive, detection of non-nucleic acid targets including whole cells. Such methods can include (a) contacting the sample with: (i) a first target binding construct to thereby immobilise or capture the target; (ii) a second target binding construct; (iii) a type V or type VI CRISPR/Cas effector protein; (iv) a trigger nucleic acid sequence; (v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and (vi) a labelled reporter construct, wherein said reporter construct comprises a nucleic acid (e.g. ssDNA, dsDNA or ssRNA depending upon the CRISPR/Cas effector utilised), does not hybridize with the guide sequence of the guide RNA and is cleavable by the nuclease activity of the activated type V or type VI CRISPR/Cas effector protein; and (b) measuring a detectable signal produced following cleavage of the labelled nucleic acid reporter by the type V or type VI CRISPR/Cas effector protein, thereby detecting the immobilised or captured target. In such a method, the type V or type VI CRISPR/Cas effector protein, the trigger nucleic acid sequence, the guide RNA, or the type V or type VI CRISPR/Cas effector protein and the guide RNA optionally in further combination with the trigger nucleic acid, is conjugated to the second target binding construct to thereby co-locate the type V or type VI CRISPR/Cas effector protein and the target (e.g. act as a bridge) when present in the sample. Alternatively, the second binding construct may be omitted and the first binding agent may instead be a conjugated binding construct having the type V or type VI CRISPR/Cas effector protein, the trigger nucleic acid sequence, the guide RNA, or the type V or type VI CRISPR/Cas effector protein and the guide RNA optionally in further combination with the trigger nucleic acid, conjugated to it.

Also described herein are enhancements to type V and VI CRISPR/Cas-mediated bio-sensing methods. More specifically, the inventors have for the first time identified chemical enhancement of the trans-cleavage rate of type V and type VI Cas effectors. Accordingly, the present invention is also directed towards type V and VI CRISPR/Cas-mediated methods for the detection of various analytes, including nucleic acid targets, wherein the trans-cleavage activity of the Cas effector(s) is enhanced through the utilisation of sulfhydryl reductants and non-ionic surfactants in the reaction mixture. Such methods can include arrangements as described above and in more detail below, or may utilise arrangements wherein a nucleic acid target is detected utilising a guide RNA designed to hybridise with the target nucleic acid.

### Type V and Type VI CRISPR/Cas Effector Proteins

Trans-cleavage based programmable nuclease: type V CRISPR/Cas systems and their effector proteins are a subtype of Class 2 CRISPR/Cas effector proteins (e.g., Cas12 family proteins such as Cas12a), see, e.g., Shmakov et al., Nat Rev Microbiol. 2017 March; 15(3):169-182: “Diversity and evolution of class 2 CRISPR-Cas systems.” Examples include, but are not limited to: Cas12 family (Cas12a, Cas12b, Cas12c), C2c4, C2c8, C2c5, C2c10, and C2c9; as well as CasX (Cas12e) and CasY (Cas12d). Also see, e.g., Koonin et al., Curr Opin Microbiol. 2017 June; 37:67-78: “Diversity, classification and evolution of CRISPR-Cas systems.” Type VI CRISPR/Cas systems and their effector proteins (e.g., Cas13 family proteins such as Cas13a), are also described see, e.g., Nat Rev Microbiol. 2017 March; 15(3):169-182: “Diversity and evolution of class 2 CRISPR-Cas systems.” Examples include, but are not limited to: Cas13 family (Cas13a, Cas13b, Cas13c). Such effector proteins are contemplated for use in the present invention.

As such in some embodiments, a subject type V CRISPR/Cas effector protein is a Cas12 protein (e.g., Cas12a, Cas12b, Cas12c). In some embodiments, a subject type V CRISPR/Cas effector protein is a Cas12 protein such as Cas12a, Cas12b, Cas12c, Cas12d, Cas12e, Cas12d, or Cas12e. In some embodiments, a subject type V CRISPR/Cas effector protein is a Cas12a protein. In some embodiments, a subject type V CRISPR/Cas effector protein is a Cas12b protein. In some embodiments, a subject type V CRISPR/Cas effector protein is a Cas12c protein. In some embodiments, a subject type V CRISPR/Cas effector protein is a Cas12d protein. In some embodiments, a subject type V CRISPR/Cas effector protein is a Cas12e protein. In some embodiments, a subject type V CRISPR/Cas effector protein is protein selected from: Cas12 (e.g., Cas12a, Cas12b, Cas12c, Cas12d, Cas12e), C2c4, C2c8, C2c5, C2c10, and C2c9. In some embodiments, a subject type V CRISPR/Cas effector protein is protein selected from: C2c4, C2c8, C2c5, C2c10, and C2c9. In some embodiments, a subject type V CRISPR/Cas effector protein is protein selected from: C2c4, C2c8, and C2c5. In some embodiments, a subject type V CRISPR/Cas effector protein is protein selected from: C2c10 and C2c9. In some embodiments, a subject type VI CRISPR/Cas effector protein is a Cas13 protein (e.g., Cas13a, Cas13b, Cas13c). In some embodiments, a subject type VI CRISPR/Cas effector protein is a Cas13 protein such as Cas13a, Cas13b, Cas13c. In some embodiments, a subject type VI CRISPR/Cas effector protein is a Cas13a protein. In some embodiments, a subject type VI CRISPR/Cas effector protein is a Cas13b protein. In some embodiments, a subject type VI CRISPR/Cas effector protein is a Cas13c protein.

In some embodiments, the subject type V or type VI CRISPR/Cas effector protein is a naturally-occurring protein (e.g., naturally occurs in prokaryotic cells). In other embodiments, the Type V or type VI CRISPR/Cas effector protein is not a naturally-occurring polypeptide (e.g., the effector protein is a variant protein, a chimeric protein, includes a fusion partner, and the like). Examples of naturally occurring Type V or type VI CRISPR/Cas effector proteins include, but are not limited to, those described in PCT/US2018/062052. Any Type V or type VI CRISPR/Cas effector protein can be suitable for the methods, compositions, kits, etc.) and methods of the present disclosure provided the Type V or type VI CRISPR/Cas effector protein forms a complex with a guide RNA and exhibits nonspecific nuclease activity of a single stranded nucleic acid reporter construct once it is activated (by hybridization of and associated guide RNA to a trigger nucleic acid sequence).

### Guide RNA

As used herein, the term “guide sequence”, “guide RNA”, “gRNA” or “guide molecule” refers to a polynucleotide comprising any polynucleotide sequence having sufficient complementarity with either a trigger nucleic acid sequence or a target nucleic acid sequence (where a nucleic acid is being detected), wherein hybridization between with guide RNA and the trigger nucleic acid sequence or target nucleic acid sequence and activate the nuclease activity of a CRISPR effector protein complexed with the guide RNA.

Where the analyte being detected is not a target nucleic acid sequence within a cell, the guide RNA and the trigger nucleic acid may each be specifically engineered and optimized for binding to each other or to the CRISPR/Cas effector protein (in the case of the guide sequence, e.g. guide RNA) or for the activation of the CRISPR/Cas effector protein since there are no constraints imparted by the specific sequence of the target to be selected. Accordingly, in some example embodiments, the degree of complementarity, when optimally aligned using a suitable alignment algorithm, is 99% or more. A guide sequence, and hence a nucleic acid-targeting guide may be selected to target any trigger nucleic acid sequence.

In another embodiment, for methods involving the detection of nucleic acid, the guide RNA is specifically engineered and optimized for binding to the desired target nucleic acid sequence.

In some embodiments, a trigger nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is rnFold, as described by Zuker and Stiegler (Nucleic Acids Res. 9 (1981), 133-148). Another example folding algorithm is the online Webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, the guide RNA comprises a stem loop, preferably a single stem loop. In certain embodiments, the direct repeat sequence forms a stem loop, preferably a single stem loop.

Effective guide RNA length, for Cas12a, requires a spacer sequence of at least 10 nucleotides to activate the nuclease function (Cell Research (2018) 28:491-493). Spacer length on gRNA can also affect reaction intensity. For example, the optimal length of spacer for Cas13b is 26 nt to 34 nt (J. S. Gootenberg et al., Science 10.1126/science.aaq0179 (2018)). The sequences of guide RNA can be modified at its terminal, or interval nucleotide. For example, the 5′ or 3′ of Cas9 crRNA modification lead to improved nuclease stability or activity.

In one embodiment the guide RNA is at least 10 nucleotides in length. In a preferred embodiment, the guide RNA sequence is 42 nucleotides in length.

The actual sequence of guide RNA can be modified at the terminal, or interval nucleotide. For example, modification of the 5′ or 3′ of Cas9 crRNA lead to improved nuclease stability or activity (McMahon et al., 2018, Moon et al., 2018, Nguyen et al., 2020).

In a preferred embodiment the guide RNA comprises the following sequence: UAA UUU CUA AGU GUA GAU GGG GGU UGG UAG GGU GUC (SEQ ID NO. 1).

In a preferred embodiment the guide RNA comprises a sequence selected from any the group consisting of SEQ ID NO. 1, UAA UUU CUA CUA AGU GUA GAU GGG GGG GGU UGG UAG GGU GUC (SEQ ID NO. 6), UAA UUU CUA CUA AGU GUA GAU GCG CAA UCA GCG UCA GUA AUG UUC (SEQ ID No. 9), UAA UUU CUA CUA AGU GUA GAU CCC AAU AAU ACU GCG UCU UGG (SEQ ID No. 12), GGA CCA CCC CAA AAA CGA AGG GGA CUA AAA CAG AGA AUU CCA UAG UCC AG (SEQ ID No. 13), GGG GAU UUA GAC UAC CCC AAA AAC GAA GGG GAC UAA AAC ACG UUG UUU UGA UCG CGC CCC (SEQ ID No. 15), or CAG AAU GGA GAA CGC AGU GGG GCG CGA UCA AAA CAA CGU CGG CCC CAA GGU UUA CCC AAU AAU ACU GCG UCU UGG UUC AC (SEQ ID No. 16).

In another embodiment, the guide RNA a nucleic acid at least one nucleotide having a different sugar backbone than the naturally occurring nucleic acids DNA or RNA. That is, at least one nucleotide containing a non-natural sugar (e.g. an XNA).

In one embodiment, the reaction ratio of the CRISPR/Cas effector protein to guide RNA ranges from 0.25 to 15.

In another embodiment, the CRISPR/Cas effector ribonucleoprotein (RNP) is present in a reaction mixture at a concentration ranging from about 0.35 ug/mL to about 55 ug/mL. In a preferred embodiment the Cas12a is present in a reaction mixture at a concentration ranging from about 0.39 ug/mL to about 50.34 ug/mL.

In another embodiment, the reaction temperature is room temperature. In another embodiment the reaction temperature ranges from about 22 degrees Celsius to about 37 degrees Celsius.

### Trigger Nucleic Acid Sequences

As described above in contrast to the primary utilization of CRISPR/Cas biosensor systems for the detection of target DNA sequences in a sample, the present invention is directed towards the detection of non-nucleic acid targets. Therefore, the nucleic acid sequences employed for the both the guide RNA and the counterpart trigger nucleic acid sequence which activates the nuclease activity of the CRISPR/Cas effector protein are not dictated by the target.

The inventors have determined that Cas12a trans-cleavage activity remains at similar level despite terminal modifications to triggering nucleic acid sequence (e.g. DNA), such as 5′ or/and 3′ attachments, or conjugation to other molecules such as antibodies (e.g. IgG protein).

Triggering dsDNA requires the TTTN PAM sequence to efficiently activate the Cas12 protein, but triggering ssDNA does not require the existence of PAM sequence (Cell Research (2018) 28:491-493). Triggering dsDNA leads to higher trans-cleavage activity (Chen et al., Science 360, 436-439 (2018), Cell Research (2018) 28:491-493).

In one embodiment, the triggering nucleic acid sequence has a length of from about 18 nucleotides to about 30 nucleotides in length. In another embodiment, the length of the triggering nucleic acid sequences is about 24 nucleotides. In a preferred embodiment, the length of the triggering nucleic acid sequences is 24 nucleotides. In another embodiment, the length of the triggering nucleic acid sequences is about 30 nucleotides. In a preferred embodiment, the length of the triggering nucleic acid sequences is 30 nucleotides. A length of triggering nucleic acid sequence of greater than 30 nucleotides may still be effective to trigger trans-cleavage of Cas protein and may not impact Cas protein activity unless detrimental secondary structures are formed by the sequence.

In one embodiment, the trigger nucleic acid sequence is a double-stranded DNA sequence or RNA sequence.

In one embodiment the trigger nucleic acid sequence comprises a double-stranded DNA sequence. In another embodiment, the trigger nucleic acid sequence comprises a single-stranded RNA sequence. In another embodiment, nucleic acid sequence the trigger comprises a double-stranded RNA sequence.

In one embodiment, the triggering nucleic acid sequence comprises a nucleic acid sequence where at least one of the nucleotides has a different sugar backbone than the naturally occurring nucleic acids DNA or RNA. That is, at least one nucleotide containing a non-natural sugar (e.g. an XNA).

In a preferred embodiment, the triggering nucleic acid sequence is single stranded DNA.

In another preferred embodiment, the triggering nucleic acid comprises the following sequence: GAA GAC ACC CTA CCA ACC CCC TAA ACC (SEQ ID NO. 2).

In another preferred embodiment, the triggering nucleic acid comprises the following sequence: GAA GAC ACC CTA CCA ACC CCC (SEQ ID NO. 3). In another preferred embodiment, the triggering nucleic acid comprises the following sequence: GAA GAC ACC CTA CCA ACC CCC CCC (SEQ ID NO. 5)

In another preferred embodiment, the triggering nucleic acid comprises the following sequence: GGA CUG GAC UAU GGA AUU CUC GGG UGC CAA GG (SEQ ID NO. 14).

In another preferred embodiment, the triggering nucleic acid comprises the following sequence: GAA GAC ACC CTA CCA ACC CCC CCC TAA ACC (SEQ ID NO. 17)

### Reporter Constructs

As used herein, a “reporter construct” refers to a molecule that can be cleaved or otherwise inactivated by an activated CRISPR system effector protein described herein and wherein such cleavage/inactivation is detectable. The term “reporter construct” may alternatively also be referred to as a “detector construct”. Depending on the nuclease activity of the CRISPR effector protein, the reporter construct may be an RNA-based construct or a DNA-based construct. The reporter construct may also be a Xeno nucleic acid (XNA) construct which includes one or more, or consists of Xeno nucleic acids or artificial nucleotides. A Xeno nucleic acid or artificial nucleotide may comprise a non-naturally occurring sugar or nucleobase. The nucleic acid-based reporter construct comprises a nucleic acid element that is cleavable by a CRISPR effector protein. Cleavage of the nucleic acid element releases the agent or produces a conformational change that allows the generation of a detectable signal. Exemplary constructs demonstrating how to use nucleic acid elements to prevent or mask the generation of a detectable signal will be known to the skilled person and exemplary embodiments are described below, and embodiments of the invention include these or variants thereof. Prior to cutting, or when the reporter construct is not in an “active” state, the reporter construct can be designed so that the generation or detection of a positive detectable signal is blocked, masked, quenched or inhibited. It will be appreciated that in certain exemplary embodiments, minimal background signal may be generated in the presence of non-active reporter constructs. The positively detectable signal can be any signal that can be detected using optical, fluorescent, chemiluminescent, electrochemical or other detection methods known in the art. The term “positive detectable signal” is used to distinguish between other detectable signals detectable in the presence of the reporter construct. For example, in certain embodiments, a first signal (i.e., a negative detectable signal) can be detected when a masking or quenching agent is present, which is then converted to a second signal (e.g., a positive detectable signal) when the target molecule is detected and the masking or quenching agent is cleaved or inactivated by the activated CRISPR effector protein.

In certain other exemplary embodiments, the reporter construct may comprise an RNA, a DNA oligonucleotide or a modified or RNA or DNA, comprising one or more Xeno Nucleic Acids (XNA) or artificial nucleotides, to which a detectable label is attached and a masking or quenching agent for the detectable label. Examples of such detectable label/masking agent pairs are fluorophores and quenchers of fluorophores. Quenching of a fluorophore can occur due to the formation of a non-fluorescent complex between the fluorophore and another fluorophore or a non-fluorescent molecule. This mechanism is called ground state complex formation, static quenching or contact quenching. Thus, an RNA or DNA oligonucleotide can be designed such that the fluorophore and quencher are sufficiently close for contact quenching to occur. Fluorophores and their associated quenchers are known in the art and can be selected by one of ordinary skill in the art for this purpose. The particular fluorophore/quencher is not critical in the context of the present invention, so long as the fluorophore/quencher pair is selected to ensure masking of the fluorophore. Upon activation of the effector proteins disclosed herein, the RNA or DNA or XNA oligonucleotides are cleaved, thereby severing the proximity between the fluorophore and quencher needed to maintain the contact quenching effect. Thus, detection of a fluorophore can be used to determine the presence of the target molecule in a sample.

In a preferred embodiment the reporter construct comprises a sequence selected from the group consisting of TTATT, UUAUU, UUUUU, TTXTT or UUXUU, where X represents an artificial nucleotide may comprise a non-naturally occurring sugar or nucleobase.

In a preferred embodiment the reporter construct is a ssRNA construct and labelled with the Fluorophore FAM (e.g. 5′) and related quencher BHQ1 (e.g. 3′). In another preferred embodiment the report construct has the sequence 5′UUAUU3′. In another preferred embodiment the foregoing ssRNA reporter construct is used in combination with Cas 12a or Cas13a.

In another preferred embodiment, the reporter construct is a ssRNA construct and labelled with the Fluorophore Texas Red (e.g. 5′) and related quencher BHQ2 (e.g. 3′). In another preferred embodiment the report construct has the sequence 5′UUAUU3′. In another preferred embodiment the foregoing ssRNA reporter construct is used in combination with Cas 12a or Cas13a.

In a preferred embodiment the reporter construct is a ssDNA construct and labelled with the Fluorophore FAM (e.g. 5′) and related quencher BHQ1 (e.g. 3′). In another preferred embodiment the report construct has the sequence 5′TTATT3′. In another preferred embodiment the foregoing ssDNA reporter construct is used in combination with Cas 12a or Cas13a.

In another preferred embodiment, the reporter construct is a ssRNA construct and labelled with the Fluorophore Texas Red (e.g. 5′) and related quencher BHQ2 (e.g. 3′). In another preferred embodiment the report construct has the sequence 5′TTATT3′. In another preferred embodiment the foregoing ssDNA reporter construct is used in combination with Cas 12a or Cas13a.

The length of RNA or DNA, or XNA oligonucleotide reporter constructs, based on design, are optimally from 4 to 15 nucleotides in length, however they may be longer. The trans cleavage activity of activated Cas 12a is random. In contrast, for different Cas13a proteins, the cutting preference is different. For example LwaCas13a has preference for U-U reporter, PsmCas13a has preference for A-A reporter, CcaCas13b has preference for U-A reporter (J. S. Gootenberg et al., Science 10.1126/science.aaq0179 (2018)).

In another embodiment, the reporter construct may be adapted for endpoint detection via a lateral flow device. The person skilled in the art will appreciate that various arrangements for a lateral flow device may be utilised in connection with the reporter constructs and methods described herein. For example, the reporter construct used in the context of the present invention comprises a first molecule and a second molecule connected by an RNA linker. The lateral flow substrate also includes a sample portion. The sample portion may be equivalent, continuous or contiguous with the reagent portion. The lateral flow strip also includes a first capture line, typically a horizontal line across the device, although other configurations are possible. The first capture area is adjacent to the sample loading portion and on the same end of the lateral flow substrate. A first binding agent that specifically binds to a first molecule of the reporter construct is immobilized or otherwise immobilized to the first capture region. The second capture area is located at an end of the lateral flow substrate opposite the first binding area. The second binding agent is immobilised or otherwise fixed at the second capture area. The second binding agent specifically binds to a second molecule of the reporter construct, or the second binding agent can bind to a detectable ligand. For example, the detectable ligand may be a particle, such as a colloidal particle, that is visually detectable when aggregated. The particles may be modified with an antibody that specifically binds to a second molecule on the reporter construct. If the reporter construct is not cleaved, the detectable ligand will accumulate at the first binding region. If the reporter construct is cleaved, the detectable ligand is released to flow to the second binding region. In such embodiments, the second binding agent is an agent capable of specifically or non-specifically binding a detectable ligand on an antibody on the detectable ligand.

For example, detection may occur via a lateral flow strip based upon degradation of a reporter construct that is labelled on opposing ends with a detection protein and biotin, respectively. The detection protein-biotinylated reporter will attach to gold nanoparticle conjugated mouse antibodies that are specific to the detection protein that are contained within a lateral flow device. If the reporter remains intact, the detection protein-biotin-labelled reporter accumulate at a first line of the strip immobilized by streptavidin (control line). In the presence of activated CRISPR/Cas effector protein (e.g. Cas 12a), i.e. when the target is present, the reporter is cleaved and freed detection protein conjugates are released to accumulate at a second line of the lateral flow strip containing anti-mouse antibody (test line).

In another preferred embodiment, the reporter construct may comprise a Xeno Nucleic Acid (XNA), or consist of XNAs. The inventors have surprisingly discovered that reporter constructs comprising certain XNAs demonstrate compatible and even enhanced performance with a DNA reporter construct.

In a preferred embodiment, the XNA included in the reporter construct is selected from deoxyuridine, 2F-RNA reporter, and 5-Aza-2′-deoxycytidine. In a preferred embodiment the report is sequence and structure: TTXTT, where X is the XNA.

In a preferred embodiment the reporter construct is a ssDNA construct and labelled with the Fluorophore FAM (e.g. 5′) and related quencher BHQ1 (e.g. 3′). In another preferred embodiment the reporter construct has the sequence 5′TTXTT3′. In a further preferred embodiment X is selected from deoxyuridine, 2F-RNA reporter, and 5-Aza-2′-deoxycytidine. In another preferred embodiment the foregoing ssDNA reporter construct is used in combination with Cas 12a or Cas13a.

In a preferred embodiment the reporter construct is a ssDNA construct and labelled with the Fluorophore Texas Red (e.g. 5′) and related quencher BHQ2 (e.g. 3′). In another preferred embodiment the reporter construct has the sequence 5′TTXTT3′. In a further preferred embodiment X is selected from the group consisting of deoxyuridine, 2F-RNA, and 5-Aza-2′-deoxycytidine. In another preferred embodiment the foregoing ssDNA reporter construct is used in combination with Cas 12a or Cas13a.

In another embodiment, the labelled reporter construct comprising a nucleic acid that can be cleaved or otherwise inactivated by an activated CRISPR system effector protein described herein, has an enzyme conjugated to the nucleic acid as the label. In such embodiments the enzyme is compatible with chromogenic, fluorogenic, and chemiluminescent substrates for generation of a detectable signal. In one embodiment, the reporter construct comprises a nucleic acid that can be cleaved or otherwise inactivated by an activated CRISPR system effector protein described herein, conjugated to a Horseradish peroxidase (HRP) or Alkaline Phosphatase (AP) enzyme. In a preferred embodiment reporter construct comprises a nucleic acid that can be cleaved or otherwise inactivated by an activated CRISPR system effector protein described herein, is conjugated to a Horseradish peroxidase (HRP). In another preferred embodiment, the enzyme conjugated nucleic acid reporter construct is also conjugated to a magnetic bead or other particle which facilities removal of uncleaved reporter constructs from a solution or reaction mixture. Such a removal step may be employed when such a reporter construct is employed for the methods described herein. In an exemplary embodiment a chromogenic, fluorogenic, and chemiluminescent substrate is added to the reaction mixture following a step of removal of magnetic beads and thereby any uncleaved reporter constructs, for the generation of a detectable signal.

In another embodiment, the reporter construct has the following structure: magnetic bead (MB)-nucleic acid-enzyme. In a preferred embodiment the reporter construct has the structure of: MB-ssDNA-HRP. In a further preferred embodiment, the nucleic acid of the enzyme-conjugated, and optionally Magnetic bead-conjugated, reporter construct comprises the sequence TTATTTTTTTTATTTTTTAT (SEQ ID NO. 4). In some embodiments, nucleic acid may comprise a tag and/or fluorescent moiety (e.g. biotin, FAM, etc). In another embodiment, the conjugation of the enzyme (e.g. HRP of AP) to the nucleic acid may occur through an enzyme labelled antibody which binds to said tag or fluorescent moiety.

### Target Binding Constructs

As used herein the term “target binding construct” refers to a construct comprising a molecule that interacts in a non-covalent fashion to a target. For example, the target binding construct may comprise a polypeptide of a known amino acid sequence capable of binding to a target of interest, usually a protein target, and usually capable of specifically binding. For example, the target binding construct can be selected to contain the amino acid sequence of the binding partner of the target protein of interest. Cell surface receptors and secreted binding proteins (such as growth factors), soluble enzymes, structural proteins (such as collagen and fibronectin), etc., as an exemplary class of target proteins for which the amino acid sequences of binding partners (such as inhibitors) are well known.

In some embodiments, the target binding construct comprises a full length antibody or an antibody fragment containing an antigen binding domain, antigen binding domain fragment or an antigen binding fragment of the antibody (e.g., an antigen binding domain of a single chain) which is capable of binding, especially specific binding, to a target of interest, usually a protein target of interest. In this embodiment the target binding construct contains an antigen binding domain. In such embodiments, the antigen binding domain can be a binding polypeptide such as, but not limited to variable or hypervariable regions of light and/or heavy chains of an antibody (VL, VH), variable fragments (Fv), F(ab′) 2 fragments, Fab fragments, single chain antibodies (scAb), single chain variable regions (scFv), complementarity determining regions (CDR), or other polypeptides known in the art containing an antigen binding domain capable of binding target proteins or epitopes on target proteins. In further embodiments, the target binding construct may be a chimera or hybrid combination containing a first target binding portion that contains an antigen binding domain and a second target binding portion that contains an antigen binding domain such that each antigen binding domain is capable of binding to the same or different target (e.g. bi-specific or multispecific antibody). In some embodiments, the target binding construct is a bispecific antibody or fragment thereof, designed to bind two different antigens. The origin of the antigen binding domain can be a naturally occurring antibody or fragment thereof, a non-naturally occurring antibody or fragment thereof, a synthetic antibody or fragment thereof, a hybrid antibody or fragment thereof, or an engineered antibody or fragment thereof.

Methods for generating an antibody for a given target are well known in the art. The structure of antibodies and fragments thereof, variable regions of heavy and light chains of an antibody (VH and VL), FV, F(ab′) 2, Fab fragments, single chain antibodies (scAb), single chain variable regions (scFv), and complementarity determining regions (CDR) are also well understood. Methods for generating a polypeptide having a desired antigen-binding domain of a target antigen are known in the art. Methods for modifying antibodies to couple additional polypeptides are also well-known in the art.

In certain embodiments, the target binding constructs employed in the methods and kits of the invention are antibodies which specifically bind to a cytokine or small molecule. In other embodiments the target binding constructs are antibodies which specifically bind to other antibodies, such as to antibodies of a different species to that of the antibody (e.g. anti-mouse-IgG, anti-rabbit-IgG etc.). In other embodiments, the target binding constructs may specifically bind to an enzyme or other label, which may themselves be employed on another target binding construct such as a peptide or antibody or a label, tag or other moiety (e.g. anti-HRP, anti-FITC etc.) which may be linked or conjugated to a peptide or antibody. The skilled person will recognise that the target binding constructs, in particular antibodies, which may be employed in the methods and kits of the invention are many and varied.

Antibodies that may be used in the methods and assays described herein include anti-Cryptosporidium antibodies such as monoclonal antibodies that bind to the surface of the oocyst wall. Alternatively, they may be polyclonal antibodies that bind to the surface of Cryptosporidium oocysts. Other antibodies that may be used in the methods and assays described herein include Anti-Giardia antibodies such as monoclonal or polyclonal antibodies that react with the surface of Giardia cysts. The antibodies may react with the Giardia cyst antigen Cyst-Wall-Protein-1 (CWP1). Giardia antibodies may be monoclonal or polyclonal antibodies that react with Giardia trophozoites.

The Cryptosporidium oocyst antigen may be detected on intact oocysts or the antigen may be released from the oocyst by stripping the antigen from the surface of the oocyst by chemical and or heat treatment. For example, the Cryptosporidium oocyst antigen may be stripped from the oocyst by boiling in 1% sodium dodecyl sulfate (SDS) for 15 minutes. The stripped antigen can then be separated from particulates by centrifuging the sample. This approach enables detection of the Cryptosporidium oocyst antigen in a sample that is free of contaminating particles.

The anti-Cryptosporidium antibodies may react with sporozoite antigens. The sporozoite antigens may be released from oocysts by chemical treatment or by performing an excystation procedure such as incubating the oocysts in acidified Hanks Bufferred Saline at 37° C. and then incubating sodium deoxycholate at 37° C. The released sporozoite antigens may be detected once the sporoziotes have infected cells in a tissue culture sample.

In certain embodiments the target binding constructs may be tagged or labelled. In one embodiment, the target binding construct is biotinylated. In another embodiment, the target binding construct is conjugated to streptavidin. In one embodiment the target binding construct is linked or conjugated to a type V or type VI CRISPR/Cas effector protein, a trigger nucleic acid sequence, a guide RNA, or a type V or type VI CRISPR/Cas effector protein in combination with the guide RNA, or a type V or type VI CRISPR/Cas effector protein in combination with the guide RNA and the trigger nucleic acid sequence. In one embodiment the target binding construct is linked or conjugated to a trigger nucleic acid sequence as described herein. In another embodiment the target binding construct is linked or conjugated to a guide RNA as described herein. In another embodiment the target binding construct is linked or conjugated to a type V or type VI CRISPR/Cas effector protein as described herein. In another embodiment the conjugation of the type V or type VI CRISPR/Cas effector protein or trigger nucleic acid sequence according to the foregoing embodiments occurs via a streptavidin-biotin interaction.

In a particular embodiment, the target binding construct is attached to solid support or substrate. An immobilized substrate may refer to any material that is suitable for, or may be modified to, the attachment of a polypeptide or polynucleotide. Possible substrates include, but are not limited to, glass and modified functionalized glass, plastic (including acrylics, polystyrene and copolymers of styrene with other materials, polypropylene, polyethylene, polybutylene, polyurethane, Teflon etc.), polysaccharides, nylon or nitrocellulose, ceramics, resins, silica or silica-based materials (including silicon and modified silicon), carbon, metals, inorganic glass, plastics, fibre optic strands, and various other polymers. In some embodiments, the solid support comprises a patterned surface suitable for immobilizing molecules in an ordered pattern. In certain embodiments, a patterned surface refers to an arrangement of distinct regions in or on an exposed layer of a solid support. In some embodiments, the solid support comprises an array of wells (e.g. a microtitre plate) or recesses in the surface. The composition and geometry of the solid support may vary depending on its use. In some embodiments, the solid support is a planar structure, such as a slide, chip, microchip and/or array. Thus, the surface of the substrate may be in the form of a planar layer. In some embodiments, the solid support comprises one or more surfaces of a flow cell. In some embodiments, the solid support or surface thereof is non-planar, such as an inner or outer surface of a tube or container. In some embodiments, the solid support comprises a microsphere or bead. “microsphere,” “bead,” “particle” is intended to mean, in the context of a solid substrate, small discrete particles made from a variety of materials including, but not limited to, plastics, ceramics, glass, and polystyrene. In certain embodiments, the microspheres are magnetic microspheres or beads. Alternatively, or additionally, the beads may be porous. The beads range in size from nanometres (e.g., 100 nm) to millimetres (e.g., 1 mm).

As described herein, the target binding constructs employed in the compositions, methods and kits of the present invention may be conjugated to a type V or type VI CRISPR/Cas effector protein, a trigger nucleic acid sequence, a guide RNA, or the type V or type VI CRISPR/Cas effector protein in combination with the guide RNA optionally in further combination with the trigger nucleic acid. In a preferred embodiment the target binding construct is conjugated to the type V or type VI CRISPR/Cas effector protein in combination with the guide RNA, wherein the type V or type VI CRISPR/Cas effector protein has been combined with or pre-loaded with the guide RNA prior to conjugation to the target binding construct.

As will be appreciated by the skilled person, there are well established methods and commercially available kits enabling the conjugation of wide variety of molecules (eg. biotin, nucleic acids, enzymes (such as Horse Radish Peroxidase (HRP)), fluorophores, etc.) to target binding constructs (e.g. antibodies) including labels. The use of such methods and materials are applicable for the generation of such conjugates described herein. In a preferred embodiment the conjugated target binding constructs described herein are generated using the methods detailed in the Examples.

### Methods

The Type V and Type VI CRISPR/Cas effector proteins, Guide RNAs, Trigger Nucleic Acid Sequences, Reporter constructs, and Target binding constructs described in embodiments above may be employed in any suitable combination in the methods described and exemplified below.

In one embodiment, the present invention provides a method for the detection of a target in a sample, the method comprising: (a) contacting the sample with: (i) a first target binding construct; (ii) a type V or type VI CRISPR/Cas effector protein; (iii) a trigger nucleic acid sequence; (v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and (iv) a labelled reporter construct, wherein said reporter construct comprises a nucleic acid that does not hybridize with the guide sequence of the guide RNA and is cleavable by the nuclease activity of the activated type V or type VI CRISPR/Cas effector protein; and (b) measuring a detectable signal produced following cleavage of the labelled nucleic acid reporter by the type V or type VI CRISPR/Cas effector protein, thereby detecting the target in the sample; wherein the type V or type VI CRISPR/Cas effector protein, the trigger nucleic acid sequence, the guide RNA, or the type V or type VI CRISPR/Cas effector protein in combination with the guide RNA optionally in further combination with the trigger nucleic acid, is conjugated to the first target binding construct to thereby co-locate the type V or type VI CRISPR/Cas effector protein and the target when present in the sample; and wherein the target is not a nucleic acid sequence. In another embodiment the first target binding construct is immobilised on a surface. In another embodiment the first target binding construct is conjugated to the type V or type VI CRISPR/Cas effector protein. In a preferred embodiment the first target binding construct is conjugated to the type V or type VI CRISPR/Cas effector protein and the guide RNA, wherein type V or type VI CRISPR/Cas effector protein has been combined with or pre-loaded with the guide RNA prior to conjugation. In a further preferred embodiment said type V or type VI CRISPR/Cas effector protein is Cas12a.

In one embodiment, the present invention provides a method for the detection of a target in a sample, the method comprising (a) contacting the sample with: (i) a first target binding construct; (ii) a second target binding construct to thereby immobilise or capture the target; (iii) a type V or type VI CRISPR/Cas effector protein; (iv) a trigger nucleic acid sequence; (v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and (vi) a labelled reporter construct, wherein said reporter construct comprises a nucleic acid that does not hybridize with the guide sequence of the guide RNA and is cleavable by the nuclease activity of the activated type V or type VI CRISPR/Cas effector protein; and (b) measuring a detectable signal produced following cleavage of the labelled nucleic acid reporter by the type V or type VI CRISPR/Cas effector protein, thereby detecting the immobilised or captured target. In such a method, the type V or type VI CRISPR/Cas effector protein, the trigger nucleic acid sequence, the guide RNA, or the type V or type VI CRISPR/Cas effector protein and the guide RNA optionally in further combination with the trigger nucleic acid, is conjugated to the first target binding construct to thereby co-locate the type V or type VI CRISPR/Cas effector protein and the target (e.g. act as a bridge) when present in the sample.

According to the foregoing embodiment, the skilled person will appreciate that the sample may be contacted with the first binding construct prior to being contacted with the second binding construct or subsequent to the sample being contacted with the second binding construct. In some embodiments the second binding construct may be immobilised on a substrate prior to coming into contact with the sample or subsequent to being contacted with said sample. For example, the second binding construct may be immobilised on a surface (e.g. of a microtitre plate) and the sample applied to that surface so as to contact the sample with the second binding construct. Subsequently, the first binding construct may be applied to the surface so as to contact the sample with the first binding construct. The type V or type VI CRISPR/Cas effector protein, trigger nucleic acid sequence, guide RNA or labelled reporter construct may, depending which of the type V or type VI CRISPR/Cas effector protein, trigger nucleic acid sequence, guide RNA or combination of type V or type VI CRISPR/Cas effector protein and guide RNA are conjugated to the first binding construct, may be contacted with the sample simultaneously with the first binding construct or subsequently. In another embodiment the first target binding construct is conjugated to the type V or type VI CRISPR/Cas effector protein. In a preferred embodiment the first target binding construct is conjugated to the type V or type VI CRISPR/Cas effector protein and the guide RNA, wherein type V or type VI CRISPR/Cas effector protein has been combined with or pre-loaded with the guide RNA prior to conjugation. In a further preferred embodiment said type V or type VI CRISPR/Cas effector protein is Cas12a.

In another embodiment, the invention provides a method for the detection of a target in a sample, the method comprising:
(a) contacting the sample with:
(i) a first target binding construct to thereby immobilise or capture the target;
(ii) a second target binding construct;
(iii) a third binding construct which binds to the second target binding construct;
(iii) a type V or type VI CRISPR/Cas effector protein;
(iv) a trigger nucleic acid sequence;
(v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
(vi) a labelled reporter construct, wherein said reporter construct comprises a nucleic acid that does not hybridize with the guide sequence of the guide RNA and is cleavable by the nuclease activity of the activated type V or type VI CRISPR/Cas effector protein; and
(b) measuring a detectable signal produced following cleavage of the labelled nucleic acid reporter by the type V or type VI CRISPR/Cas effector protein, thereby detecting the immobilised or captured target;
wherein the type V or type VI CRISPR/Cas effector protein, the trigger nucleic acid sequence, the guide RNA, or the type V or type VI CRISPR/Cas effector protein and the guide RNA, optionally in further combination with the trigger nucleic acid, is conjugated to the third binding construct to thereby co-locate the type V or type VI CRISPR/Cas effector protein and the target when present in the sample; and wherein the target is not a nucleic acid sequence.

In an embodiment the third target binding construct is conjugated to the type V or type VI CRISPR/Cas effector protein. In a preferred embodiment the third target binding construct is conjugated to the type V or type VI CRISPR/Cas effector protein and the guide RNA, wherein type V or type VI CRISPR/Cas effector protein has been combined with or pre-loaded with the guide RNA prior to conjugation. In a further preferred embodiment said type V or type VI CRISPR/Cas effector protein is Cas12a.

In another embodiment, the invention provides a method according to the aspects and embodiments described and exemplified herein, further comprising a step of conjugating the type V or type VI CRISPR/Cas effector protein, the trigger nucleic acid sequence, the guide RNA, or the type V or type VI CRISPR/Cas effector protein and the guide RNA optionally in further combination with the trigger nucleic acid, to said target binding construct prior to use. In an embodiment the target binding construct is conjugated to the type V or type VI CRISPR/Cas effector protein. In a preferred embodiment the target binding construct is conjugated to the type V or type VI CRISPR/Cas effector protein and the guide RNA, wherein type V or type VI CRISPR/Cas effector protein has been combined with or pre-loaded with the guide RNA prior to conjugation. In a further preferred embodiment said type V or type VI CRISPR/Cas effector protein is Cas12a.

In another embodiment, the target may be immobilised on a substrate or expressed on the surface of a cell.

In a further embodiment, the invention provides a method for the detection of a nucleic acid target in a sample, comprising: (a) contacting the sample with a reaction mixture comprising: (i) a type V CRISPR/Cas effector protein (ii) a guide RNA comprising: a region that binds to the type V or type VI CRISPR/Cas effector protein, and a guide sequence that hybridizes with the target nucleic acid sequence, wherein hybridization between the guide sequence and the target nucleic acid sequence activates the nuclease activity of the CRISPR/Cas effector protein; (iii) a labelled reporter construct, wherein said reporter construct is a ssRNA acid that does not hybridize with the guide sequence of the guide RNA and is cleavable by the nuclease activity of the activated type V or type VI CRISPR/Cas effector protein; and iv) a sulfhydryl reductant, and/or v) a non-ionic surfactant; and (b) measuring a detectable signal produced following cleavage of the labelled nucleic acid reporter by the type V CRISPR/Cas effector protein, thereby detecting the target in the sample.

As described herein the inventors have surprisingly found that the trans-cleavage efficiency of both Type V and type VI Cas effector proteins can be improved with chemical enhancers. Optimisation of trans-cleavage rates have previously focused on modification of crRNA in some instances leading to 2-3.5 fold increase in trans-cleavage activity. The inventors have discovered that certain sulfhydryl reductants, including Dithiothreitol (DTT), or Tris(2-carboxyethyl) phosphine (TCEP), when added to biosensing systems incorporating type V and type VI Cas effectors enhance the trans-cleavage rates of Cas 12a and Cas13a. Furthermore, certain non-ionic surfactants, including Brij L23 and poly(vinyl alcohol) (PVA), were also observed to have a similar effect. However, the inventors have surprisingly discovered that a combination of DTT and PVA is capable of yielding a synergistic increase of trans-cleavage activity of Cas12a and Cas13a of greater than 5 fold. Such enhancement is capable of substantially increasing the detection sensitivity of detection systems utilising Cas 12a and Cas13a and decrease reaction times.

In a particularly preferred embodiment, the reaction mixture comprises said sulfhydryl reductant, and said non-ionic surfactant.

In one embodiment, the sulfhydryl reductant is selected from Dithiothreitol (DTT), or Tris(2-carboxyethyl) phosphine (TCEP) to and 2-Mercaptoethanol (2-ME)). In a preferred embodiment the sulfhydryl reductant is DTT. In a further preferred embodiment DTT is provided at a concentration ranging from 100 μM to 20 mM. In a further preferred embodiment DTT is provided at a concentration of 10 mM for Cas12a, and at 5 mM for Cas13).

As detailed herein, the inventors have identified that in particular embodiments, the rate of signal production can be substantially increased through the addition of DTT alone, and further augmentation can occur when the reaction is carried out at about 37° C.

Accordingly, in a preferred embodiment of the foregoing methods, DTT is added to the reaction mixture and where reduction of time for the production of a signal is required the reaction is preferably carried out at about 37° C.

In one embodiment, the non-ionic surfactant is selected from Brij L23 and poly(vinyl alcohol) (PVA). In a preferred embodiment the non-ionic surfactant is PVA. In a further preferred embodiment PVA is 87-90% hydrolyzed, average mol wt 30,000-70,000. In a further preferred embodiment PVA is provided at a concentration ranging from 0.33% to 3.3%. In a further preferred embodiment PVA is provided at a concentration of 1% for Cas12a and 0.05% for Cas13a.

In another embodiment of the detection of the nucleic acid target comprises an RT PCR step or an amplification step.

In one embodiment, the invention provides a method of enhancing a type V or Type VI CRISPR/Cas detection system comprising adding a sulfhydryl reductant, and/or a non-ionic surfactant to a reaction mixture comprising the type V or Type VI CRISPR/Cas effector of the system.

In a further embodiment, the method of enhancing a type V or Type VI CRISPR/Cas detection system comprises adding a sulfhydryl reductant, and a non-ionic surfactant to a reaction mixture comprising the type V or Type VI CRISPR/Cas effector of the system. In one embodiment, the sulfhydryl reductant is selected from Dithiothreitol (DTT), or Tris(2-carboxyethyl) phosphine (TCEP) to and 2-Mercaptoethanol (2-ME)). In a preferred embodiment the sulfhydryl reductant is DTT. In a further preferred embodiment DTT is provided at a concentration ranging from 100 μM to 20 mM. In a further preferred embodiment DTT is provided at a concentration of 10 mM for Cas12a, and at 5 mM for Cas13). In one embodiment, the non-ionic surfactant is selected from Brij L23 and poly(vinyl alcohol) (PVA). In a preferred embodiment the non-ionic surfactant is PVA. In a further preferred embodiment PVA is 87-90% hydrolyzed, average mol wt 30,000-70,000. In a further preferred embodiment PVA is provided at a concentration ranging from 0.33% to 3.3%. In a further preferred embodiment PVA is provided at a concentration of 1% for Cas12a and 0.05% for Cas13a. In a preferred embodiment, the method of enhancing a type V or Type VI CRISPR/Cas detection system comprises adding DTT and PVA.

According to another aspect, the present invention provides a method of modifying an immunoassay comprising replacing a labelled target binding construct to be employed for signal generation in said immunoassay with a replacement target binding construct directed to the same target; and a) contacting the sample with a reaction mixture comprising: i) said replacement target binding construct; ii) a type V or type VI CRISPR/Cas effector protein; (iii) a trigger nucleic acid sequence; (iv) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and (v) a labelled reporter construct, wherein said reporter construct comprises a nucleic acid that does not hybridize with the guide sequence of the guide RNA and is cleavable by the nuclease activity of the activated type V or type VI CRISPR/Cas effector protein; and (b) measuring a detectable signal produced following cleavage of the labelled nucleic acid reporter by the type V or type VI CRISPR/Cas effector protein, thereby detecting the immobilised or captured target;

wherein the type V or type VI CRISPR/Cas effector protein, the trigger nucleic acid sequence, the guide RNA, or the type V or type VI CRISPR/Cas effector protein and the guide RNA, optionally in further combination with the trigger nucleic acid, is conjugated to the replacement binding construct to thereby co-locate the type V or type VI CRISPR/Cas effector protein and the target when present in the sample; and wherein the target is not a nucleic acid sequence.

In another embodiment, the invention provides a method for the detection of a target in a sample, the method comprising: (a) contacting the sample with: (i) a first target binding construct; (ii) a type V or type VI CRISPR/Cas effector protein; (iii) a trigger nucleic acid sequence; (v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and (iv) a labelled reporter construct, wherein said reporter construct comprises a nucleic acid that does not hybridize with the guide sequence of the guide RNA and is cleavable by the nuclease activity of the activated type V or type VI CRISPR/Cas effector protein; and (b) measuring a detectable signal produced following cleavage of the labelled nucleic acid reporter by the type V or type VI CRISPR/Cas effector protein, thereby detecting the target in the sample; wherein the type V or type VI CRISPR/Cas effector protein, the trigger nucleic acid sequence, the guide RNA, or the type V or type VI CRISPR/Cas effector protein in combination with the guide RNA optionally in further combination with the trigger nucleic acid, is conjugated to the first target binding construct to thereby co-locate the type V or type VI CRISPR/Cas effector protein and the target when present in the sample; and wherein the target is not a nucleic acid sequence.

### Kits

Also provided herein is a kit comprising one or more of: a first binding construct; a type V or type VI CRISPR/Cas effector protein; a trigger nucleic acid sequence; a guide RNA; a labelled reporter construct as described in various embodiments hereinabove. In one embodiment, the kit further comprises one or more of: a medium, buffer, reagent comprising a sulfhydryl reductant and/or a non-ionic surfactant, a reagent for fluorometric, chemiluminescent or colorimetric detection, apparatus or other component that can be used in a method described herein, and instructions for use, for example instructions on how to perform an assay, and a vial or other container for housing one of these aforementioned components.

### EXAMPLES

### Example 1. Generation of a CRISPR/Cas-Based Biosensing System

### Materials and Reagents

High binding plate (Corning, 9018), DNase/RNase free water (ThermoFisher), Phosphate Buffered Saline (PBS) (Sigma, 10 mM, pH=7.4), streptavidin (Sigma), Crypto incubation buffer (Biopoint Pty Ltd), Crypto wash buffer (Biopoint Pty Ltd), Customised CRISPR buffer (CC buffer: 1×PBS buffer, 0.04% Tween20, 10 mM MgCl2, 10 μg/mL BSA), inactivated Crypto (Biopoint Pty Ltd), Tex-Crypto (Biopoint Pty Ltd), Crypto antibody (Cry104, mouse IgG; Biopoint Pty Ltd) acting as Crypto capture antibody, FITC-labelled Cry104 acting as Crypto detection antibody tests, anti-mouse IgG antibody (NL637), biotin conjugation kit (ABCAM), streptavidin conjugation kit (ABCAM), EnGen® Lba Cas12a (Cpf1) protein (New England Biolab), 10×NEB 2.1 buffer (New England Biolab), All RNA and DNA oligos were synthesized and modified by Sango Biotech.

TABLE 1
Nucleic acid sequences used in Example 1

| | Oligo | | Length | Modifi- |
|---|---|---|---|---|
| Name | type | Sequence 5′-3′ | (nt) | cation |
| Guiding | ssRNA | UAA UUU CUA | 42 | N/A |
| RNA | | AGU GUA GAU | | |
| (gRNA) | | GGG GGU UGG | | |
| | | UAG GGU GUC | | |
| | | (SEQ ID No. 1) | | |
| Triggering | ssDNA | GAA GAC ACC | 30 | 5′- |
| ssDNA | | CTA CCA ACC | | biotin |
| | | CCC TAA ACC | | |
| | | (SEQ ID No. 2) | | |
| ssDNA | ssDNA | TTATT | 5 | 5′-Texas |
| reporter | | | | Red; |
| | | | | 3′-BHQ2 |

### Fabrication of Abs-ssDNA Conjugates

The streptavidin conjugated Crypto antibodies were synthesized according to a commercially available streptavidin conjugation kit (ABCAM). First, 1 μL modifier was mixed with 10 μL (1 mg mL-1) antibody (66.7 μmol) gently. Then 10 μL (1 mg mL-1) streptavidin (189.4 μmol) was added into the solution and gently mixed at room temperature for 3 h. Finally, 1 μL quencher reagent was gently mixed into the solution for 30 min at room temperature. Afterwards, the biotinylated triggering ssDNA was applied to form the Abs-ssDNA composites followed by mixing of 10 μL 10 μM (100 μmol) biotinylated triggering ssDNA with 1 μg prepared streptavidin conjugated antibody (6.7 μmol) for 3 h at room temperature, then the solution was centrifugation-filtered using low binding PES filter with 10 k molecular separation pores to remove the unattached antibody. The spin was set as 12,000 rpm for 5 mins. PBS buffer washing was applied three times, and the collected Abs-ssDNA composites were stored at 4° C. for further use.

### Fabrication of Biotinylated Crypto Antibody

The biotinylated Crypto antibodies were synthesized according to the instruction in the biotin conjugation kit from ABCAM. First, 1 μL modifier was mixed with 10 μL (1 mg/mL) antibody (66.7 μmol) gently. Then 10 μL biotin solution was added into the antibody mixture and gently mixed at room temperature for 3 h. Finally, 1 μL quencher reagent was gently mixed into the solution for 30 min at room temperature. The prepared biotinylated Crypto antibody was stored at 4° C. for further use.

### Preparation of CRISPR/Cas12a Reaction Mixture

The CRISPR/Cas12a reaction mixture was prepared as follows: 10 μL of 10 μM (100 μmol) Cas12a protein was gently mixed with 5 μL of 20 μM (100 μmol) gRNA in 5 mL 1×NEB 2.1 buffer, which was diluted by DNase/RNase free water. Then, 10 μL of 100 μM (1 nmol) ssDNA linked fluorescent reporter was added and well mixed to form the final reaction mixture.

### Preparation of Deactivated Crypto Samples

Following deactivation of fresh live Crypto samples involving heat inactivation applied at 90° C. for 15 min, a final Crypto stock solution was stained with 10 μL of 1 mg/mL propidium iodide (37° C., 5 min) for the evaluation of viability. The prepared sample was analysed on flow cytometer using forward scatter (FSC) and FL-2 detectors. The final demonstrated deactivated Crypto samples were used in the examples described herein.

The Crypto samples with specified number of Crypto microorganisms were prepared and identified on a sorting flow cytometer using forward scatter (FSC) and side scatter (SSC) detectors. The population of Crypto was gated by using with the FSC and FL-2 detectors. Defined number of Crypto microorganisms were dispensed to individual test tubes.

### The Development of Plate Based CRISPR/Cas Biosensing Assay

The plate based CRISPR/Cas biosensing assay was developed stepwise as below. The high binding plate was rinsed with PBS buffer to clean the wells. Then 100 μL of 10 μg/mL streptavidin solution was added in each well of the high binding plate to create a streptavidin-modified interface. After 1 hour incubation at room temperature (RT), the excess streptavidin molecules were washed away using 350 μL of a PBS buffer. Subsequently, 350 μL of Crypto incubation buffer was applied to each well for 1 hour at RT for the blocking of empty spots. 5 μg/mL biotinylated Crypto antibody was applied to the well blocked plates to generate the biosensing interface. The Crypto sample was then added to the antibody modified plate for 1 hour incubation at RT, then the non-specifically attached Crypto were washed away by using the Crypto wash buffer. After capturing the Crypto, 5 μg/mL of antibody-ssDNA conjugate was applied for 1 hour at RT to form the sandwich structure, and the residual unbound conjugates was washed away using the Crypto wash buffer. Finally, 100 μL CRISPR/Cas solution was added into each well and incubated for 30 min before testing the fluorescence signal. The fluorescent signal was detected using ID3 plate reader several times at 30 mins intervals with excitation wavelength of 570 nm and emission wavelength of 615 nm.

### The Characterization of Crypto Using Confocal Microscopy

In all confocal microscopy imaging, the same parameters were applied: intensity 3%, PMT 500 volts, 20 magnification, 630×630 μm2 field of view, offset 3%, 1.0 pinhole. The excitation wavelength for Tex-Crypto was 561 nm, and the emission range for Tex-Crypto was 570-670 nm. The excitation wavelength for FAM-antibody was 488 nm, and the emission range for FAM-antibody was 500-540 nm.

### Results

### 1. Assay Principle

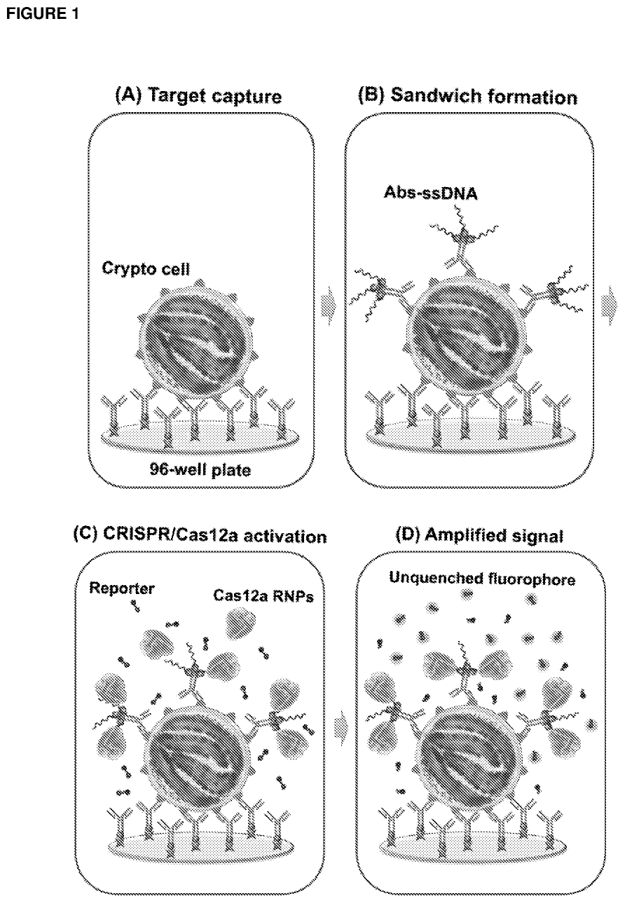

FIG. 1 shows the basic schematics of the principle of the CRISPR/Cas12a-based whole cell level biosensing for Cryptosporidium microorganisms. After the sensing interface has been prepared on the surface of a high-binding 96-well plate coated with streptavidin and biotinylated anti-Cryptosporidium antibody, the Cryptosporidium oocysts in the sample are captured onto the sensing interface (FIG. 1 A). Then, pre-made anti-Cryptosporidium antibodies and single strand DNA (Abs-ssDNA) conjugates are added to recognize and bind to the remaining free anti-Cryptosporidium antigens on the surface of the captured Cryptosporidium oocyst (FIG. 1 B). These two steps realise a standard “sandwich” immunoassay scheme where the Cryptosporidium oocysts act as the “analyte”; this is possible because of the much larger size of Cryptosporidium oocyst (˜4 μm) compared to the antibodies. Afterwards, the CRISPR/Cas12a reaction mixture with the guide RNA integrated with the CRISPR/Cas12a RNP having a complementary sequence to the triggering ssDNA on the Ab-ssDNA conjugate is introduced. This complementarity allows this RNP to be specifically activated by the recognition of the triggering ssDNA by the Ab-ssDNA conjugate (FIG. 1 C). An abundant supply of quenched ssDNA-based fluorescent reporters is also provided (FIG. 1 C). The activated CRISPR/Cas12a RNPs then indiscriminately and continuously cut the surrounding reporter molecules, owing to their collateral cleavage property upon RNP activation by the conjugate. This, in turn, unquenches the ssDNA-based fluorescent reporters and generates the amplified fluorescence signal required for analyte detection (FIG. 1 D). In this way our sensing scheme is able to take advantage of the highly efficient collateral cleavage activity of the programmable Cas12a nuclease which is triggered by an antibody-cell sandwich immunoassay.

### 2. Cryptosporidium Sample Preparation and Cell Morphology Test

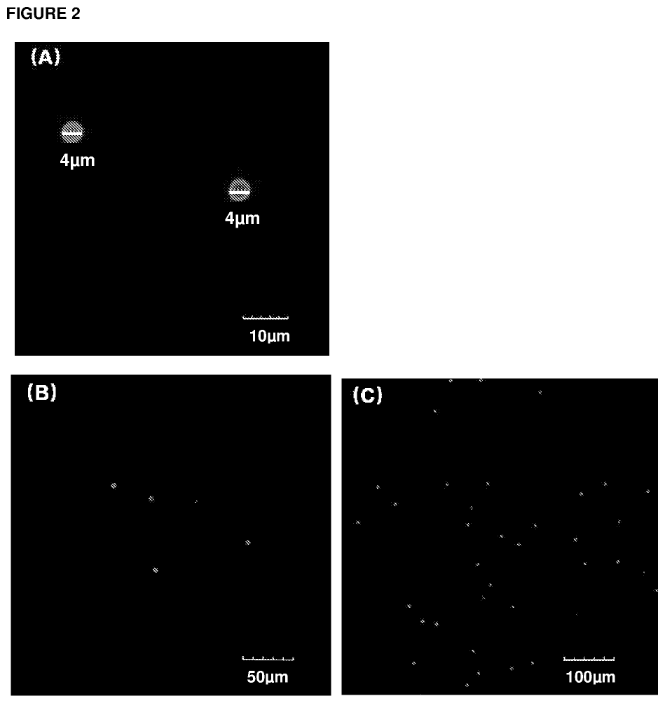

Deactivated (no longer viable) Texas Red-labelled Cryptosporidium oocysts and native Cryptosporidium oocysts (Biopoint Pty Ltd) were employed. Oocysts in the deactivated Cryptosporidium samples have been visualised by confocal microscopy (FIG. 2), which shows the expected Cryptosporidium oocyst size of ˜4 μm. The Cryptosporidium oocysts in saline solution have no tendency for clustering (FIG. 2), which is beneficial for quantitative immunoassay detection. Stock samples with concentration of 1.6×10⁶ cells/mL of deactivated Cryptosporidium have been prepared in saline solution. Additionally, 200 μL 1×PBS samples with precise number of Cryptosporidium oocysts (0, 1, 5, 10 oocysts) have also been prepared by using a sorting flow cytometer. All prepared Cryptosporidium samples were stored at 4° C. for later use.

### 3. Validating the Affinity of Anti-Cryptosporidium Antibody to Cryptosporidium Oocysts

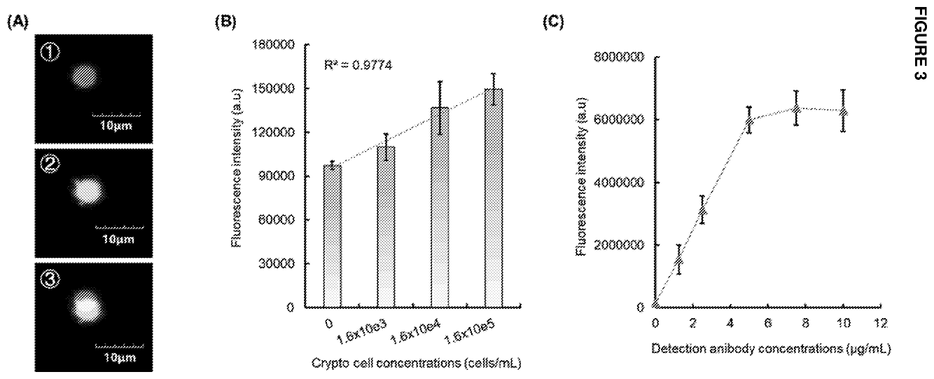

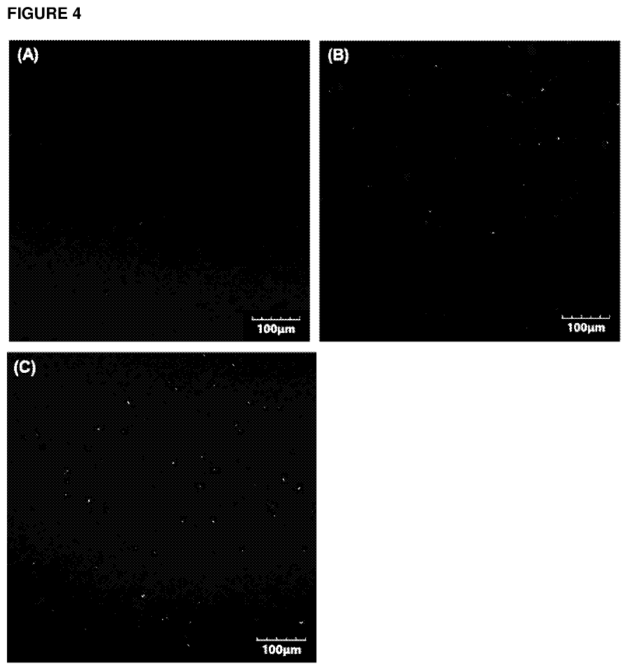

A FITC-labelled anti-Cryptosporidium antibody has been used to verify the binding affinity to Cryptosporidium cell surface. To help locate and visualize the binding of antibody on the cell surface, Texas Red-labelled Cryptosporidium oocysts have been mixed with the FITC-labelled anti-Cryptosporidium antibody which produces a green fluorescence signal. Confocal microscopy imaging at 60× magnification shows a green fluorescent signal colocalised with red fluorescent signal from the Texas Red-coated Crypto oocysts (FIG. 3A), which indicates specific binding of the anti-Cryptosporidium antibody to the Cryptosporidium cell surface antigens. In addition, as the Cryptosporidium oocyst concentration increases, a positive correlation with the FITC signal intensity has also been observed, which further confirms the antibody binding (FIG. 4).

### 4. Forming the Anti-Cryptosporidium Antibody-Cell Sandwich Structure

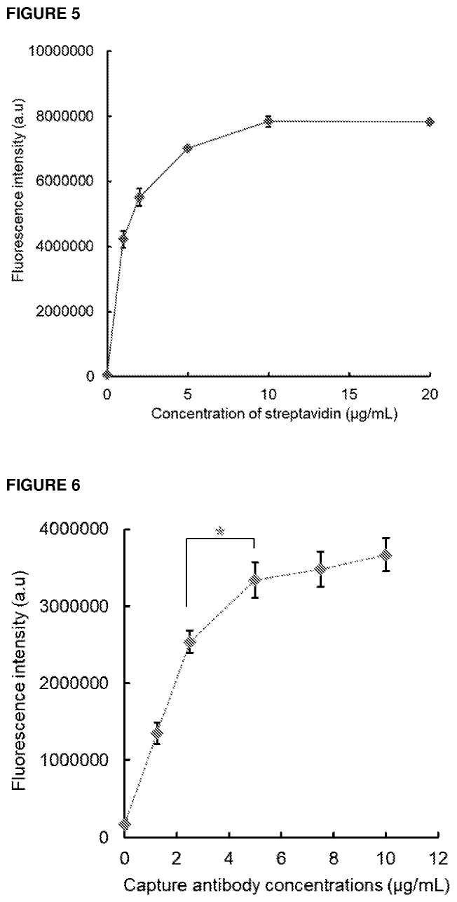

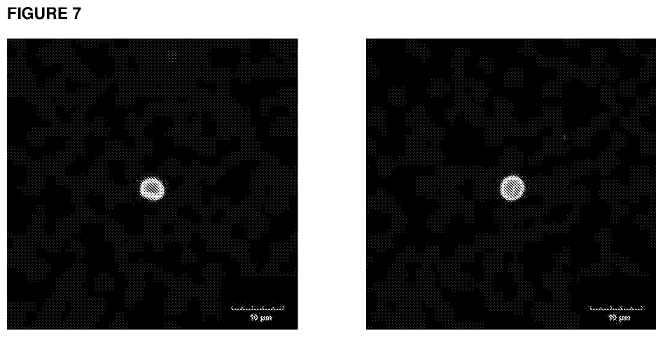

Firstly, the basic sensing interface has been established on a high-binding 96 well plate surface. The plate was coated with streptavidin at the optimal concentration of 10 μg/mL (FIG. 5). Then the biotinylated anti-Cryptosporidium antibody prepared by a commercial biotin-antibody conjugation kit has been applied to this streptavidin-coated surface to form the target capture interface. The formation of the capture interface has been verified by applying the anti-mouse IgG antibody (NL637) to recognize the anti-Cryptosporidium antibody on the sensing interface. An increased florescent signal has been observed with the increase of the biotinylated anti-Cryptosporidium antibody concentration, which, eventually, saturated at 5 μg/mL (FIG. 6). In this way, the inventors verified a successful immobilization of the biotinylated anti-Cryptosporidium antibody onto the high-binding surface, and found that 5 μg/mL is an optimum coating concentration. Subsequently, the capability to capture Cryptosporidium oocysts by this sensing interface has been investigated by applying different concentrations of Texas Red-coated Cryptosporidium samples (0, 1.6×103, 1.6×104, 1.6×105 oocysts/mL). A positive linear correlation was found between the Cryptosporidium oocysts concentrations and Texas Red fluorescence intensity (FIG. 3B), which confirmed a successful capture of target Cryptosporidium oocysts onto the established sensing interface, along with the capability to differentiate concentration changes. With a relatively large cell size of the Cryptosporidium oocysts (˜4 μm) and uniform distribution of anti-Cryptosporidium antigens across the oocyst surface (FIG. 7), it is reasonable to expect that the same anti-Cryptosporidium antibody can be used both as the capture antibody and detection antibody for a whole microorganism-based sandwich immunoassay. To verify this, a FITC-labelled anti-Cryptosporidium antibody was applied as the detection antibody after a high concentration of Cryptosporidium oocysts have been captured on the sensing interface. With increasing concentration of the FITC-labelled anti-Cryptosporidium antibody, the fluorescent signal intensity correspondingly increased, and it reached the saturation level at the antibody concentration of 5 μg/mL (FIG. 3C). These data confirm the successful formation of the expected sandwich structure, and indicate the optimum concentration of the detection antibody.

### 5. CRISPR/Cas12a-Based Signal Transduction and Amplification

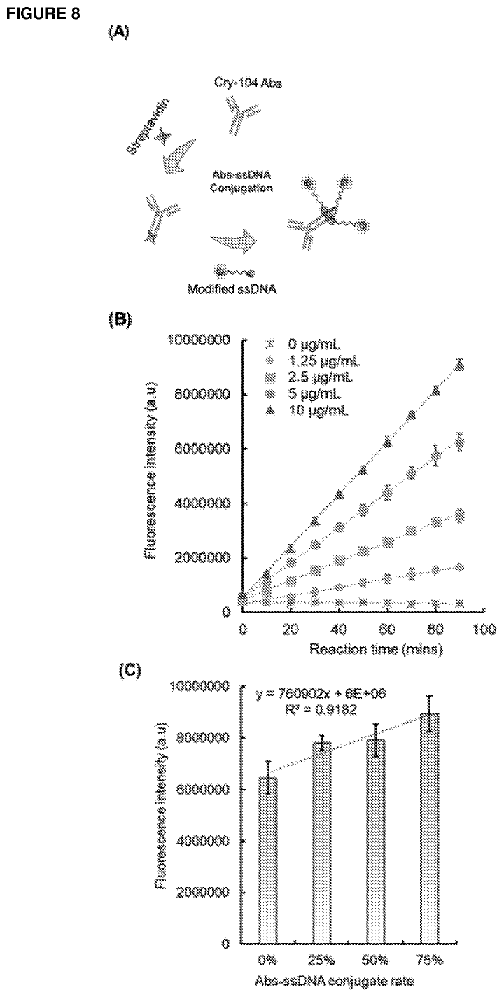

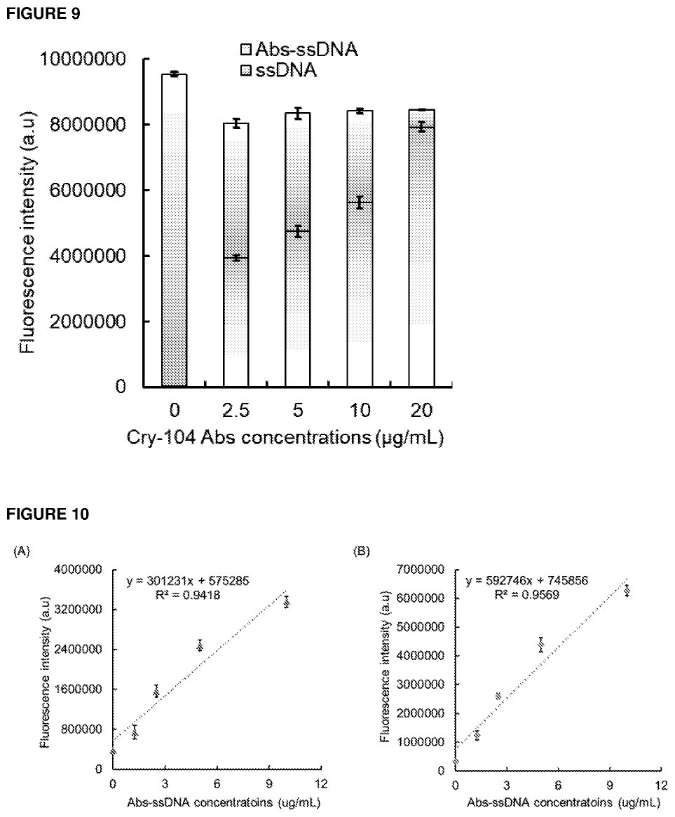

Cas12a is a programmable nuclease that can be activated by a DNA molecule with a sequence that is complementary to its guide RNA. This recognition event then triggers sequence-nonspecific cleavage of the surrounding ssDNA. Therefore, for a successful deployment of a CRISPR/Cas12a effector system to detect the presence of Cryptosporidium oocyst, a bridge or co-location between Cryptosporidium oocyst recognition and CRISPR/Cas12a RNP activation is critical. This has been realized here by conjugating the anti-Cryptosporidium antibody with single strand DNA molecules which are complementary to the RNA guide. In FIG. 8 the inventors illustrate the procedures used for synthesis of the Abs-ssDNA conjugate. A specifically designed 24 nt short ssDNA oligonucleotide was first synthesized with 5′ labelled biotin and 3′ labelled Texas Red fluorophore, which acts as the “triggering ssDNA” for the downstream CRISPR/Cas12a RNPs. In order to conjugate this ssDNA oligonucleotide to the antibody, streptavidin was directly linked onto the antibody primary amine groups by using a commercial conjugation kit. Subsequently, the biotinylated triggering ssDNA was attached onto the antibody through streptavidin-biotin binding, forming a functional Abs-ssDNA conjugate. After centrifugation-based filtration by using a low binding PES membrane (10 kD), the excess unbound ssDNA oligonucleotides were separated and removed due to their significantly lower molecular weight compared to the antibody. As here the fluorescence signal only came from the 3′ labelled Texas Red fluorophore, the fluorescence signal changes between the remaining and filtered samples represents the binding of ssDNA to the antibody. With the increase of the antibody concentration, the increased fluorescence signal (3′ labelled Texas Red fluorophore from the attached ssDNA) of the expected Abs-ssDNA conjugate indicated the successful formation of the Abs-ssDNA conjugates (FIG. 9).

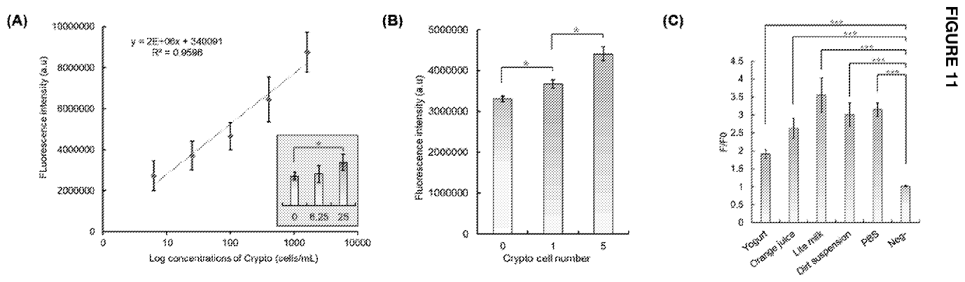

The ability of the antibody-based Abs-ssDNA conjugate to trigger the CRISPR/Cas12a collateral cleavage activity is critical for generating the final amplified fluorescent signal. The inventors evaluated the interaction between our anti-Cryptosporidium Abs-ssDNA conjugate and its corresponding CRISPR/Cas12a RNPs with the guide RNA sequence complementary to the triggering ssDNA. To this aim, different concentrations (0, 1.25, 2.5, 5 and 10 μg/mL) of Abs-ssDNA conjugate (where triggering ssDNA was conjugated without 3′ Texas Red labelling) have been mixed with the CRISPR/Cas12a reaction mixture. Compared to the sample without the Abs-ssDNA conjugate, significantly increased fluorescent signals observed for all Abs-ssDNA concentrations demonstrated a successful activation of the CRISPR/Cas12a nuclease activity (FIG. 8B). Notably, the Abs-ssDNA conjugates at different concentrations produced CRISPR/Cas12a-based signal amplification with a close linear correlation between the fluorescent intensity and the quantity of Abs-ssDNA. This reveals the potential to realise quantitative detection with this Abs-ssDNA conjugate (FIGS. 8B, FIG. 10).

A competitive immunoassay has also been performed to investigate the change of the binding affinity of the ssDNA-modified anti-Cryptosporidium antibody in comparison to the unmodified one. To this aim, the Cryptosporidium oocysts have been captured at the sensing interface, and the antibody mixtures with different proportions of the prepared Abs-ssDNA conjugates and unmodified antibody have been applied to form the antibody-microorganism sandwich structure. The final fluorescent signal was then generated by the activation of CRISPR/Cas12a RNP by the triggering ssDNA of the Abs-ssDNA conjugate in the sandwich. With the increase of the proportion of the Abs-ssDNA conjugate in different antibody mixtures (from 0% to 75% of the Abs-ssDNA conjugate) the fluorescent signal has also increased, and a positive linear correlation (R²=0.92) has been found between signal intensity and the proportion of Abs-ssDNA conjugate (FIG. 8C). This indicates that the binding affinity of the Abs-ssDNA conjugate has not been significantly compromised through conjugation with the triggering ssDNA.).

### 6. Performance of the CRISPR/Cas12a-Based Whole Cell Immunosensor for Cryptosporidium Detection

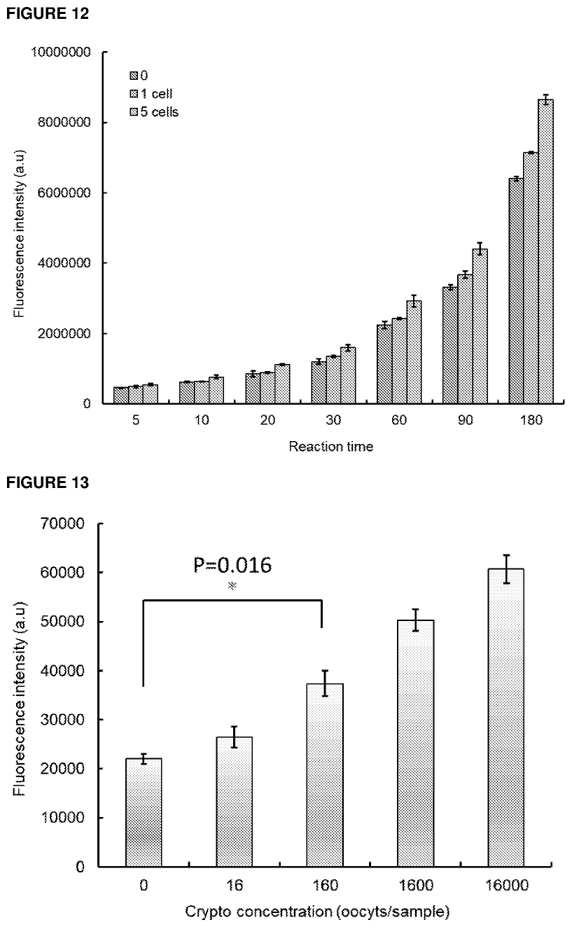

With the plate-based sensing interface, the Abs-ssDNA conjugate and the CRISPR/Cas12a reaction system prepared as described above, the performance of the CRISPR/Cas12a-based whole microorganism immunoassay has been investigated. The inventors applied this assay for Cryptosporidium oocyst detection with different microorganism concentrations, using 4× dilution from stock concentration of 1600 oocysts/mL. The inventors found that with the increase of the Cryptosporidium concentration from 6.25 oocysts/mL to 1600 oocysts/mL, a positive linear correlation is observed between pathogen concentration and the fluorescence intensity, with linear range spanning 3 orders of magnitude, and with correlation coefficient R²=0.9596 (FIG. 11A). Notably, as 100 μL of each Cryptosporidium sample has been used here for detection, the sensitivity observed in these samples was within the range of single Cryptosporidium oocyst per sample at the lowest 6.25 oocysts/mL concentration. Therefore, to further verify the ability of our assay for single Cryptosporidium oocyst detection, samples with precise numbers of individually dispensed Cryptosporidium oocysts prepared on a sorting flow cytometer (0, 1, 5 microorganisms per sample) were tested. As expected, it was observed that the fluorescence signal increases with the increase of reaction time for all tested samples, and statistically significant differences between different Cryptosporidium concentrations in fluorescence intensity were observable after 60 mins (FIG. 12). Eventually, after a reaction time of 90 mins, the fluorescent signal intensity of the sample containing 1 Cryptosporidium oocyst had become significantly higher when compared to the sample with no Cryptosporidium oocysts, which demonstrates the single-microbe level detection in our system (FIG. 11B). In comparison, Texas Red labelled anti-Cryptosporidium antibody was also used to replace the CRISPR/Cas12a-based signal amplification for fluorescence signal measurement, and the data shown 2-logs decrease in sensitivity down to 160 microorganisms per sample (FIG. 13). Reaching single microorganism level sensitivity is particularly important for screening pathogens that have extremely low infectious dose, such as Cryptosporidium.

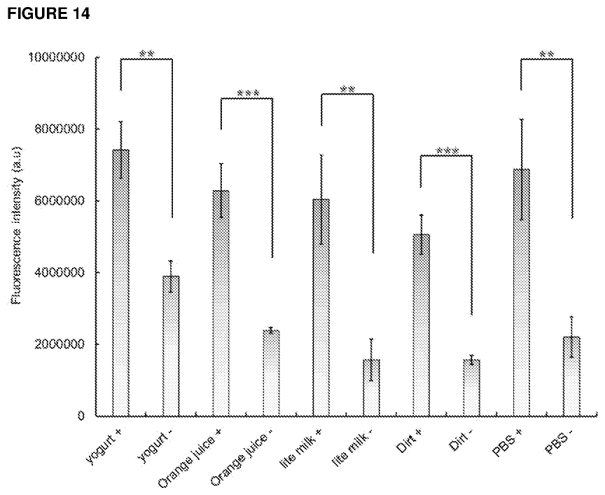

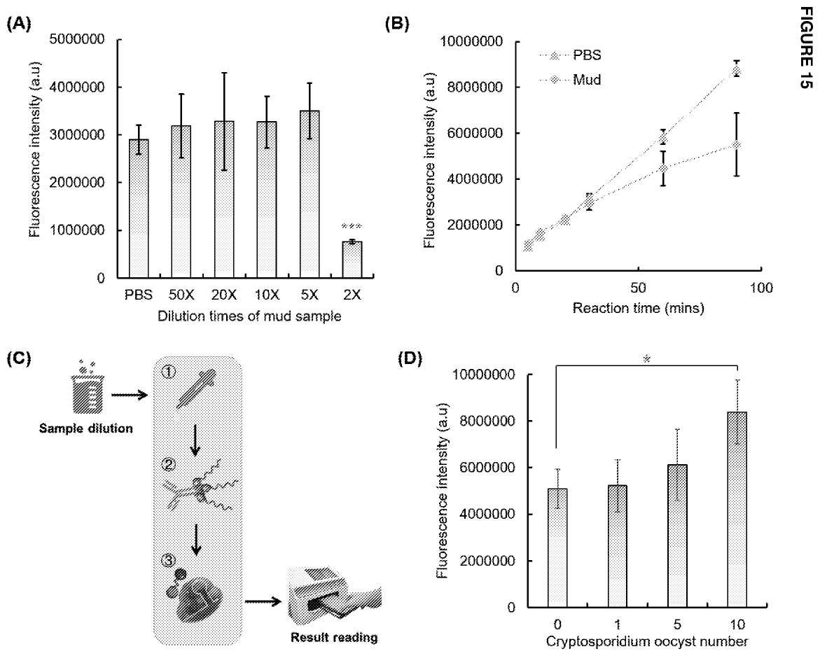

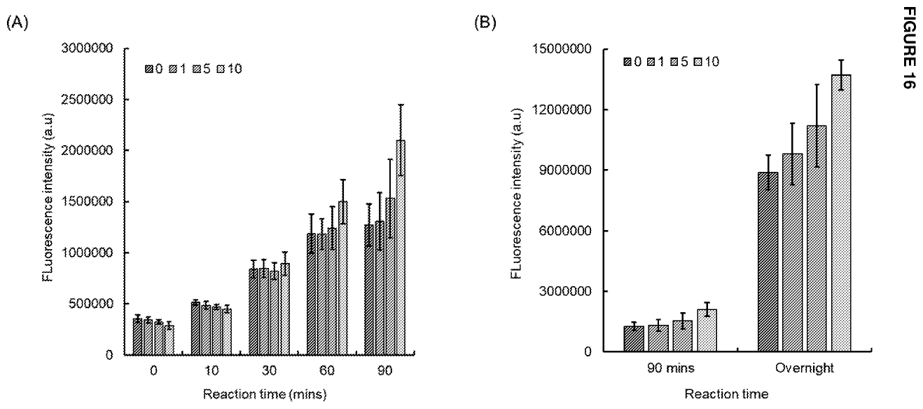

The system selectivity has also been investigated by comparing assay performance in 4 different types of complex sample matrices (yogurt, orange juice, lite milk and dirt suspension), which are representative for real testing environments commonly found in food or water monitoring. After each of these 4 matrix samples were diluted 10 times in 1×PBS buffer, Cryptosporidium oocysts were added to a final concentration of 800 oocysts/mL on average (80 oocysts per 100 μl sample), and then compared with control samples containing the same concentration of Cryptosporidium oocysts in PBS. The results have shown significantly higher fluorescent signal intensities in all tested sample matrices in the presence of Cryptosporidium oocysts (FIG. 11C and FIG. 14), which indicate satisfactory system selectivity. It can thus be concluded that even in complex sample environments, the methods of the present invention can specifically respond to the presence of its particular target and differentiate Cryptosporidium oocysts from various interferences commonly found within these matrix types.

Additionally, to assess the performance of our CRISPR/Cas12a-based biosensing system in comparison to the established Cryptosporidium detection methods, the currently widely used magnetic bead-based Cryptosporidium oocysts isolation followed by microscopy inspection and cell counting, has also been performed. The test results indicated that the magnetic beads approach can reach sensitivity of ˜62.5 oocysts/mL with two orders of magnitude linear range from 32.15 to 1000 oocysts/mL. The relatively lower performance of the magnetic bead method is tentatively attributed to higher non-specific absorption of antibody to the beads than to our plate. In comparison, our CRISPR/Cas12a-based system has a superior sensitivity reaching down to 1 single Cryptosporidium oocyst, and it also has a slightly wider linear range than the magnetic beads method. More importantly, the accuracy and sensitivity of results and data interpretation in the microscopy-based method strongly depends on operator experience, while our system uses a simple experimental protocol, practically identical to the common ELISA assays, and with no need for expensive microscopy and special training.

### 7. Deploying the Biosensing System for Cryptosporidium Detection in Water Treatment Samples

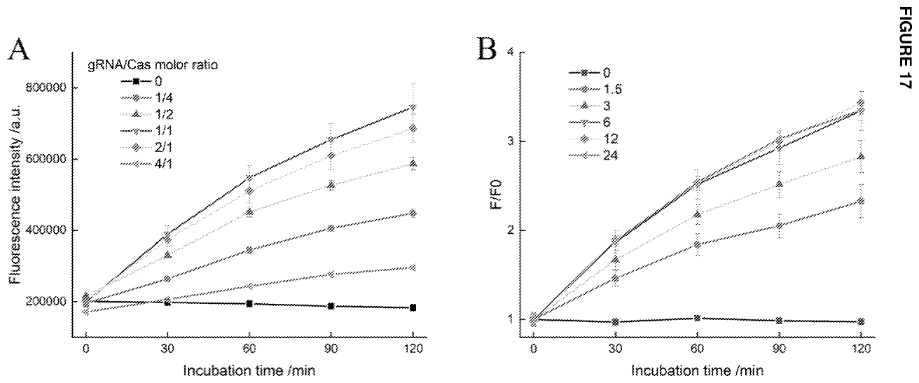

Samples from water treatment plants are commonly used in environmental surveillance or water quality control and here the inventors demonstrated the potential application of our CRISPR/Cas-based whole cell biosensing system for directly detecting Cryptosporidium oocysts from the filter back-wash mud samples from a water treatment plant with no complicated sample pre-treatment. Firstly, the tolerance of the system described herein to such mud samples has been tested by using diluted mud samples without spiked Cryptosporidium. It is reasonable to expect that these mud samples contain no native Cryptosporidium. Compared to background fluorescence signal generated in the PBS sample, the diluted mud samples with no added Cryptosporidium show no significant changes in their fluorescence levels between 5 to 50 times dilution (FIG. 15A). The fluorescent signal intensity significantly drops when the mud sample is diluted by a factor of 2. Therefore, sample dilution by at least 5-fold, is critical for our system to overcome sewage interferences.

To test the sewage for the presence of Cryptosporidium, a precise number of 5 Cryptosporidium oocysts have been spiked into each sample. It was found that in contrast to pristine PBS samples, testing of mud samples produces a significantly lower fluorescent signal intensity, particularly when the CRISPR/Cas12a reaction time is longer than 30 mins (FIG. 15B). With increasing reaction time, the difference in fluorescence intensity between these two types of samples also became more pronounced, along with significantly higher variations for the mud sample (FIG. 15B). Although the biosensing system remained functional in the mud samples, loss of signal intensity and reduced system signal intensity may indicate that the complex sample matrix in mud samples could potentially suppress the CRISPR/Cas12a enzymatic activity. In this case extended reaction time may help to compensate the signal intensity loss (FIG. 15B).

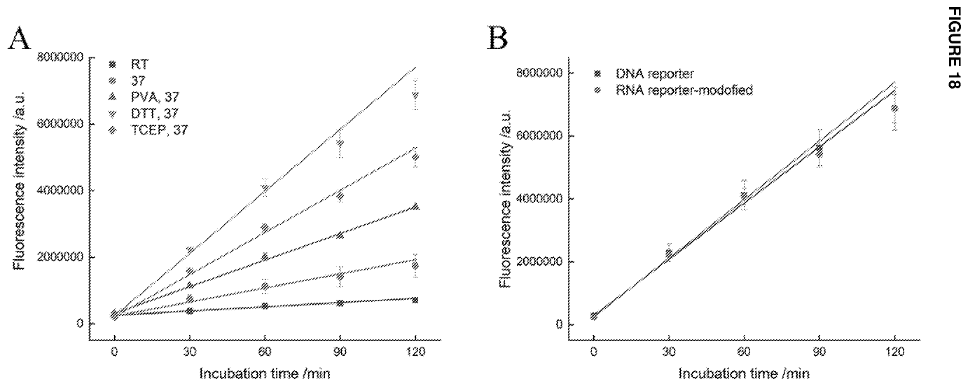

After the system has been successfully demonstrated in the mud samples with appropriate dilution pre-treatment, the performance for differentiating low levels of Cryptosporidium oocysts has been investigated. Here, the same ELISA-like protocol has been used with an additional sample dilution procedure to generate 10× diluted mud samples with precise Cryptosporidium oocysts numbers (0, 1, 5, 10 oocysts per sample, FIG. 15C). After 90 mins CRISPR/Cas12a reaction for signal generation and amplification, the results showed the expected positive correlation between Cryptosporidium numbers and fluorescent signal intensities (FIG. 15D). We found that only the mud samples with 10 Cryptosporidium oocysts were able to yield a statistically different signal intensity compared to negative controls (FIG. 15D). The overnight reaction time was also investigated to explore its effect on system sensitivity. With the reaction time increased to overnight, an improved linear correlation has been observed between 0 and 10 cells, but statistical analysis still did not show a significant difference compared with the results of a 90 mins reaction (FIG. 16).

### Discussion

The programmable recognition of nucleic acid sequences by Cas proteins has also been successfully applied in biosensing. Nucleic acid detection with remarkable sensitivity, specificity and speed has clearly demonstrated the potential of CRISPR/Cas systems for novel biosensor development and recently, CRISPR/Cas-based biosensing have been expanded to include various non-nucleic acid targets, such as protein, small molecules, ions. However, unlike the recognition of nucleic acid targets that can directly be carried out by the CRISPR/Cas RNPs, an intermediate component is needed to bridge the recognition of non-nucleic acid targets and the CRISPR/Cas activity.

The formation of the antibody and DNA hybrid, the Abs-ssDNA conjugate introduced in this work for the first time, has allowed us to create a simple and universal bridge to recognize the target and enable the generation of the amplified assay signal. The triggering ssDNA on the Abs-ssDNA conjugate acts as a nucleic acid target for CRISPR/Cas12a RNP, where the sequence-dependent specific recognition allows the RNP to become a transducer and amplifier to generate the final fluorescent signal for detection.

Advantageously, in the system described for the first time herein, between target cell recognition and generation of the final fluorescence signal, a 3-level signal amplification cascade has been implemented. Its stages include binding of multiple Abs-ssDNA conjugates to the surface of an individual microbe because of large oocyst surface area, multiple triggering ssDNA attached to one anti-Cryptosporidium antibody due to the multiple binding channel of conjugated streptavidin, and enzymatic activity of the Cas nuclease where one activated CRISPR/Cas12a RNP cleaves multiple nucleic acid reporters by its collateral cleavage activity. These three stages enhance one another, allowing the system to achieve single microbe sensitivity.

In contrast to other methods for CRISPR/Cas biosensing for microbial detection, which are focused on microbial nucleic acid recognition, the system developed by the present inventors targets surface antigens, which is an important alternative indicator of the specific microorganisms. Furthermore, the methods of the present invention do not require additional nucleic acid amplification methods, such as the RPA (Recombinase Polymerase Amplification) in SHERLOCK or PCR (Polymerase Chain Reaction) in HOLMES, to reach a satisfactory detection sensitivity. This is important because in nucleic acid detection approaches, the presence of targeted fragment of genomic material may be accompanied by nucleic acid contamination, which does not necessarily indicate the existence of functional microorganisms. In contrast, the antigen-based test described for the first time herein can provide evidence of whole microbial structures. More importantly, targeting antigens can also provide an advantage in the investigation of certain surface virulence factors in pathogen detections, such as the type 1 fimbrial adhesin, which has been identified on the tips of the enterotoxigenic E. coli fimbrial. Additionally, directly targeting whole microorganisms eliminates the need for lysis and DNA/RNA extraction, which are essential for nucleic acid based approaches, and are time consuming and inefficient and can lead to additional target loss or contamination.

Currently, Immune-Magnetic Separation (IMS) and immunofluorescence staining followed by microscopy examination is the gold-standard for Cryptosporidium detection (R. M. Chalmers, F. Katzer, Trends Parasitol 29(5) (2013) 237-51; Hassan, E., et. al., Talanta 222 (2021); J. L. Sinclair, Enumeration of Cryptosporidium spp. in water with US EPA method 1622, USA, J AOAC Int 83(5) (2000) 1108-14). A definitive positive diagnostic of the Cryptosporidium oocysts in this method requires microscopic identification which can be challenging due to its small size (˜4 μm), their scarcity in environmental samples, variation in recovery rate (Hassan, E., et. al., Talanta 222 (2021)) and poor reproducibility especially in wastewater samples (L. Xiao, K. A. Alderisio, J. Jiang, Detection of Cryptosporidium oocysts in water: effect of the number of samples and analytic replicates on test results, Appl Environ Microbiol 72(9) (2006) 5942-7). Microscopic identification requires trained experts and the whole process is time consuming and labour intensive. It is not suitable for large-scale screening or implementation using automatic platforms, which can be extremely valuable for daily safety surveillance in food or water industry or fast response to outbreaks. As an alternative approach, immunoassay-based methods for targeting Cryptosporidium antigens have also been demonstrated, but they require high concentrations of Cryptosporidium oocysts (with detection limit generally ranging between 10 to 30 oocysts), so they are more suitable to clinical samples and not to testing environmental samples such as water.

In conclusion, in this work the inventors have successfully targeted complex whole microbial structures using a CRISPR/Cas-associated detection assay for the first time. As a new ultrasensitive pathogen detection approach, the assay permits the flexibility on account of a sandwich immunoassay setup, excellent specificity, and short assay time. Surprisingly, the assay also reached single pathogen level sensitivity without significant increase of diagnostic cost. By changing the antibodies, the approach has the potential to be applied to a variety of different targets, and as an isothermal detection approach it is suitable for low resource settings. With the potential for transferring the sensing interface onto solid surfaces, such as paper-based material or polymer-based membranes, this technology can also represent a valuable approach towards better Point-of-Care diagnostics and rapid detection of pathogens relevant in public health.

### Example 2. RNA Reporter-Based CRISPR/Cas12a Biosensing System for the Detection of Single Cryptosporidium

### Materials and Methodology

EnGen® Lba Cas12a (Cpf1) protein (New England Biolab), 10×NEB 2.1 buffer (New England Biolab), Biotinylation Kit/Biotin Conjugation Kit-Lightning-Link® (Abcam, ab201796), Streptavidin Conjugation Kit—Lightning-Link® (Abcam, ab102921), High binding plate (Corning, 9018), Phosphate Buffered Saline (PBS) (Sigma, 10 mM, pH=7.4), streptavidin (Sigma), Crypto incubation buffer (provided by Biopoint Pty Ltd), EasyStain wash buffer (provided by Biopoint Pty Ltd), anti-Cryptosporidium antibody (monoclonal mouse IgG, Biopoint Pty Ltd.), Milli-Q water.

All nucleotide oligos were synthesized by Sangon Co. Ltd. with designed terminal modifications.

TABLE 2
Sequences Oligos used in Example 2.
Sequences were synthesized by Sangon Co. Ltd.

| | Oligo | | Length | Modifi- |
|---|---|---|---|---|
| Name | type | Sequence 5′-3′ | (nt) | cation |
| Guiding | ssRNA | UAA UUU CUA | 42 | N/A |
| RNA | | AGU GUA GAU | | |
| (gRNA) | | GGG GGU UGG | | |
| | | UAG GGU GUC | | |
| | | (SEQ ID No. 1) | | |
| Triggering | ssDNA | GAA GAC ACC | 24 | 5′- |
| ssDNA | | CTA CCA ACC | | biotin |
| | | CCC CCC | | |
| | | (SEQ ID No. 5) | | |
| ssRNA | ssRNA | UUAUU | 5 | 5′-FAM; |
| reporter | | | | 3′-BHQ1 |

### Preparation of Standard CRISPR/Cas12a Reaction Mixture

10 μL 10 μM (100 μmol) Cas12a protein was gently mixed with 5 μL 20 μM (100 μmol) gRNA in 3.6 mL 1×NEB 2.1 buffer, which was diluted by Milli-Q water. Then, 6 μL 100 μM (0.6 nmol) ssRNA linked fluorescent reporter was added and well mixed to form the final reaction mixture. The prepared reaction mixture was kept on ice before use.

### Optimization of RNA Reporter Based CRISPR/Cas12a System

The optimization of gRNA concentration was based on the standard CRISPR/Cas12a reaction mixture. The concentration of Cas12a effector was fixed at 10 μL 10 μM (100 μmol) and the concentration of RNA reporter was fixed at 0.6 nmol, while the concentration of gRNA was changed from 25, 50, 100, 200, and 400 pmol. After preparation of the reaction mixture, 5 μL 1 μM trigger DNA was added into 100 μL standard CRISPR/Cas12a and incubate for 120 min.

The optimization of RNA reporter concentration was based on the standard CRISPR/Cas12a reaction mixture. The concentration of Cas12a effector and gRNA were fixed at 100 μmol, while the concentration of RNA reporter was changed from 150, 300, 600, 1200, and 2400 pmol. After preparation of the reaction mixture, 5 μL 1 μM trigger DNA was added into 100 μL standard CRISPR/Cas12a and incubate for 120 min.

### Optimization of Chemical-Based Reaction Buffer.

Three different chemicals were applied in the reaction buffer system, and the concentration of 1,4-dithiothreitol (DTT) was 10 mM, the concentration of Tris(2-carboxyethyl)phosphine hydrochloride (TCEP) was 0.5 mM, and the concentration of Polyvinyl alcohol (PVA) was 10 mg/mL.

### Synthesis the Abs-ssDNA Conjugate Using Anti-Cryptosporidium Antibody

Firstly, the pristine anti-Cryptosporidium antibody (Cry104) was conjugated with streptavidin according to the instruction in the antibody-streptavidin conjugation kit (Abcam, ab102921). Briefly, 1 μL modifier was mixed with 10 μL (1 mg/mL) antibody (66.7 μmol) gently. Then 10 μL (1 mg/mL) streptavidin (189.4 μmol) was added into the solution and gently mixed at room temperature for 3 h. 1 μL quencher reagent was then gently mixed into the solution for 30 min at room temperature. Afterwards, 10 μL 10 μM (100 μmol) commercially synthesized biotinylated triggering ssDNA (either with or without 5′ Texas Red labelling) was mixed with 1 μg prepared streptavidin conjugated antibody (6.7 μmol) for 3 h at room temperature. Then the mixture was centrifugation-filtered at 12,000 rpm at 4° C. for 5 mins using low binding 10 kD PES filter (PALL) to remove the unbound free triggering ssDNA. 1×PBS buffer washing was applied 1 times, and the collected Abs-ssDNA conjugate was resuspended to 10 μg/mL concentration in 1×PBS buffer and stored at 4° C. for further use.

### The Detection of Single Crypto Using RNA Reporter Based CRISPR/Cas12a System

First, add 100 μL 10 μg/mL streptavidin PBS solution into the wells of 9018 high-binding plate, and incubate at room temperature for one hour. Then wash the plate using 350 μL PBA buffer, twice.

Second, add 350 μL incubation buffer into each well at room temperature for one hour. Then remove the incubation buffer.

Third, add 100 μL 10 μg/mL biotinylated Cry-104 antibody into each well at room temperature for one hour. Then wash the plate using 350 μL EasyStain wash buffer, twice.

Fourth, add 200 μL Crypto samples with different numbers (0, 1, 2, 5) into each well and incubate for 1 hour at room temperature, then wash with 350 μL EasyStain wash buffer, twice.

Fifth, add 100 μL 10 μg/mL antibody-ssDNA conjugate into each well at room temperature for one hour. Then wash the plate using 350 μL EasyStain wash buffer, twice.

Sixth, add 100 μL standard CRISPR/Cas12a reaction solution into each well and incubate for 120 min at room temperature.

Finally, test the fluorescence signal by iD3 plate reader with excitation wavelength of 480 nm and emission wavelength of 520 nm (RNA reporter).

### Results

### Optimization of RNA Reporter Based CRISPR/Cas12a System

In order to make the RNA reporter based CRISPR/Cas12a system work as good as the DNA reporter based CRISPR/Cas12a system, the concentrations of gRNA and RNA reporter were firstly investigated. As shown in FIG. 17A, the concentration of Cas12a effector was fixed at 100 μmol, then various gRNA concentrations were applied (25, 50, 100, 200, and 400 μmol). The optimum gRNA concentration was verified at 100 μmol, and the molar ratio of gRNA to Cas12a effector was 1:1, which indicates that one Cas12a effector with one gRNA sequence was the optimum combination.

In addition to the gRNA, the fluorescent reporter was another significant effector for CRISPR/Cas biosensing system. As shown in FIG. 17B, the concentrations of Cas12a effector and gRNA were fixed at 100 μmol, then a variety of fluorescent RNA reporter were applied (150, 300, 600, 1200, and 2400 μmol). The relative fluorescence was investigated and compared in FIG. 17B. After the molar ratio of RNA reporter to Cas12a effector reached 6 times, the relative fluorescence no longer increased, which indicates that 6 times of RNA reporter was the saturate point for current CRISPR/Cas system.

After optimization of the basic components of CRISPR/Cas12a system, the investigation of temperature and various chemicals in the standard NEB2.1 buffer were investigated. As shown in FIG. 18A, the influence of temperature was first investigated, and the performance of CRISPR/Cas12a biosensing system was better at 37° C. than that at room temperature (RT). In order to further enhance the performance of CRISPR/Cas12a biosensing system, various chemicals were added in the standard reaction buffer NEB2.1. And DTT added CRISPR/Cas12a biosensing system exhibits the best performance.

As known, the conventional CRISPR/Cas12a system exhibits much better trans-cleavage ability to DNA sequence than RNA sequence. In order to verify the optimized RNA reporter based CRISPR/Cas12a biosensing system, the conventional DNA reporter based CRISPR/Cas12a system was applied as control in FIG. 18B. Compatible performance of modified RNA reporter based CRISPR/Cas12a system was observed with DNA reporter based CRISPR/Cas12a system. Therefore, modified RNA reporter based CRISPR/Cas12a system was an alternative option for conventional CRISPR/Cas biosensing system.

### Application of Optimized RNA Reporter Based CRISPR/Cas12a System for the Detection of Crypto.

The mechanism for the detection of Crypto using RNA reporter based CRISPR/Cas12a system is as shown in FIG. 1. In order to immobilize intense capture antibodies on the high binding plate, 10 μg/mL streptavidin was first immobilized, then 350 μL incubation buffer was applied for blocking. 10 μg/mL biotinylated Cry104 antibody was then applied to form the capture sensing interface. After capture of the target Crypto, Abs-ssDNA was applied to form the sandwich structure, then RNA reporter based CRISPR/Cas solution was applied for fluorescent signal readout.

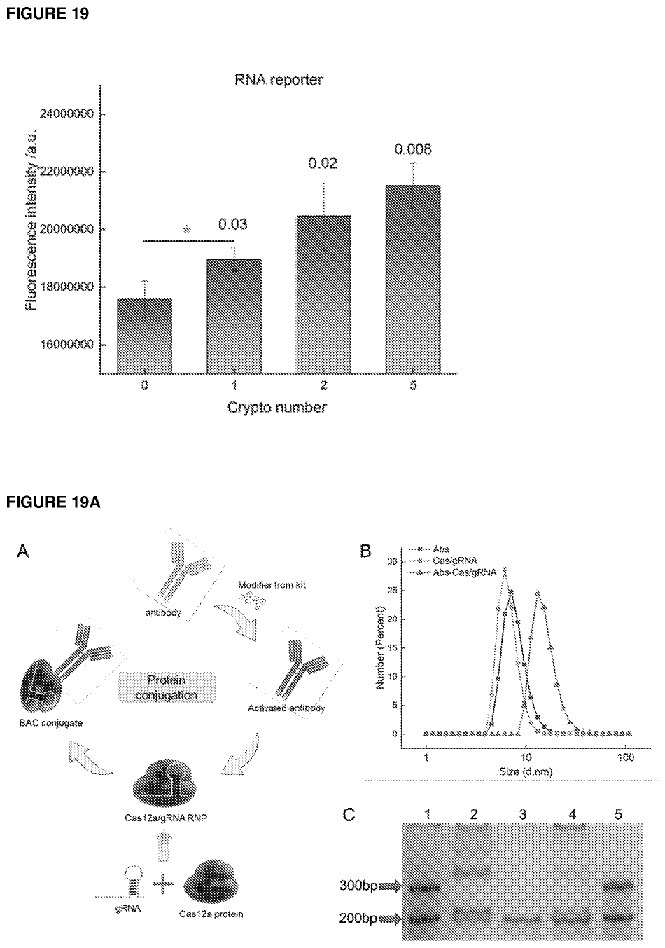

As shown in FIG. 19, various Crypto samples with exact number (0, 1, 2, 5 in 200 uL saline sample) were applied for the evaluation of the optimized RNA reporter based CRISPR/Cas12a biosensing system. After incubation two hours of the CRISPR/Cas solution, significant fluorescence difference was observed. The fluorescence signal linearly increased with the increase of Crypto numbers, and the calculated P value between single Crypt and 0 was 0.03, which indicates that the modified RNA reporter based CRISPR/Cas biosensing system is able to detect single Crypto.

### Conclusion

In this research, the inventors have successfully developed a modified RNA reporter based CRISPR/Cas12a system. The concentrations of gRNA and RNA reporter were first investigated, and 100 pmol gRNA and 600 pmol RNA reporter were the optimum conditions. Then various chemicals in the standard NEB2.1 reaction buffer were investigated, and the best performance of CRISPR/Cas12a biosensing system was observed at 10 mM DTT at 37° C., which has compatible performance to DNA reporter based CRISPR/Cas12a biosensing system. Finally, the optimized RNA reporter based CRISPR/Cas12a biosensing system was applied to detect the actual Crypto samples, and single Crypto was detected. Therefore, this modified RNA reporter based CRISPR/Cas12a system provides an alternative approach for the application of CRISPR/Cas12a biosensing system for the measurement of diverse nucleic acids, small molecule and small proteins.

### Example 3. Detection of Cryptosporidium in a Water Sample

### Spiking and Concentration of River Water

A spike dose containing 1 Cryptosporidium oocyst was prepared by flow cytometry as described previously (Reynolds DT, Slade RB, Sykes NJ, Jonas A, Fricker CR. Detection of Cryptosporidium oocysts in water: techniques for generating precise recovery data. J Appl Microbiol. 1999 December; 87(6):804-13. doi: 10.1046/j.1365-2672.1999.00862.x. PMID: 10664905). The spike dose was added to a 20 litre sample of river water.

The sample was concentrated by filtration through a mixed-cellulose ester (cellulose acetate-cellulose nitrate) membrane filter (diameter, 142 mm) with a pore size of 3 μm within a stainless steel filter housing equipment (Millipore Australia Pty., Ltd., New South Wales, Australia).

The membrane was removed from the filter housing and washed in a plastic dish by the addition of 20 ml of PBS plus 0.1% Tween 80. A stainless steel paint scraper was then used to scrape the surface of the membrane. Washings from this procedure were collected into a 200-ml centrifuge tube. The procedure was repeated three times, and the eluates from all washes were combined and centrifuged at 1,100×g for 15 min.

### Immunomagnetic Separation

The concentrated water sample was purified using immunomagnetic separation. Magnetic beads coated with anti-Cryptosporidium monoclonal antibody (Biopoint Pty Ltd). The water sample was placed into a Leighton tube and 100 μl of the IMS beads were added. The sample was rotated at room temperature for 30 minutes and then placed into a magnetic tube holder (TCS Biosciences, UK) and left for 2 minutes. The sample was then tipped off. The tube was then removed from the magnet and the beads were eluted from the side of the tube and transferred to an Eppendorf tube. The Leighton tube was rinsed with PBS plus 0.1% Tween 80 and the rinsing volume added to the Eppendorf tube.

The Eppendorf was placed in a magnetic tube holder and the supernatant was removed with a pipette leaving behind a bead pellet.

The bead pellet was then assayed using the methods described in the foregoing examples.

### Example 4. Antibody-Cas Conjugates for the Ultrasensitive Detection of Single Cryptosporidium

### Materials and Methodology

EnGen® Lba Cas12a (Cpf1) protein (New England Biolab), 10×NEB 2.1 buffer (New England Biolab), Streptavidin Conjugation Kit—Lightning-Link® (Abcam, ab102921), High binding plate (Corning, 9018), Phosphate Buffered Saline (PBS) (Sigma, 10 mM, pH=7.4), streptavidin (Sigma), Crypto incubation buffer (Biopoint Pty Ltd), EasyStain wash buffer (Biopoint Pty Ltd), Crypto and ColorSeed Texas red labelled Cryptosporidium (Biopoint Pty Ltd), anti-Cryptosporidium antibody (Cry104 monoclonal mouse IgG, Biopoint Pty Ltd.), 9018 high binding plate (Corning, Life Science), and Milli-Q water.

All nucleotide oligos were synthesized by Sangon Co. Ltd. with designed terminal modifications.

TABLE 3
Oligos used in Example 4.
Sequences were synthesized by Sangon Co. Ltd.

| | Oligo | | Length | Modifi- |
|---|---|---|---|---|
| Name | type | Sequence 5′-3′ | (nt) | cation |
| Guiding | ssRNA | UAA UUU CUA | 42 | N/A |
| RNA | | CUA AGU GUA | | |
| (gRNA) | | GAU GGG GGG | | |
| | | GGU UGG UAG | | |
| | | GGU GUC | | |
| | | (SEQ ID No. 6) | | |
| Triggering | ssDNA | GAA GAC ACC | 24 | 5′- |
| ssDNA | | CTA CCA ACC | | biotin |
| | | CCC CCC | | |
| | | (SEQ ID No. 5) | | |
| ssDNA | ssDNA | TTATT | 5 | 5′-Texas |
| reporter | | | | Red; |
| | | | | 3′-BHQ2 |

### Synthesis of the Cry104 Antibody-Cas12a/gRNA Conjugate

Firstly, 2 uL 100 μM (30 ug) Cas12a protein and 10 uL 20 μM gRNA were mixed in 30 uL NEB2.1 buffer to form the Cas12a/gRNA mixture solution. In the meanwhile, 3 uL modifier from the streptavidin conjugation kit (Abcam, ab102921) was mixed with 23 uL 1.3 mg/mL (30 ug) Cry104 antibody gently. Then, the two types of solutions were mixed together and incubated for 3 hours at room temperature. After the incubation, 3 uL quencher from the streptavidin conjugation kit (Abcam, ab102921) was added into the solution to stop the excess conjugation reaction. Finally, 50% glycerol (volume) was added into the prepared antibody-Cas12a/gRNA solution for long time storage in −20° C. freezer.

For comparison, an antibody-Cas12a conjugate was prepared according to the same procedure, where only Cas12a protein was mixed with the Cry104 antibody and modifier (i.e. without gRNA).

Evaluation of the Trans-Cleavage Ability of Cry104-Cas12a/gRNA Conjugate with ssDNA Reporters

The standard CRISPR/Cas12a mixture was first prepared using Cry104-Cas12a/gRNA conjugate. Briefly, 100 pmol of Cry104-Cas12a/gRNA conjugate was added into 3.6 mL 1×NEB 2.1 buffer. Then, 6 μL 100 μM (0.6 nmol) ssDNA linked fluorescent reporter and 36 μL 1 M DTT were added and well mixed to form the final reaction mixture. The prepared reaction mixture was kept on ice before use.

In order to evaluate the trans-cleavage ability of Cry104-Cas12a/gRNA conjugate, 5 μL 1 μM triggering ssDNA was added into 100 μL prepared standard CRISPR/Cas12a mixture and incubated for 120 min at room temperature or 37° C. before fluorescence signal test using ID3 plate reader (Excitation wavelength of 570 nm and emission wavelength of 615 nm).

### Evaluation of the Recognition Ability of Cry104-Cas12a/gRNA Conjugate

The recognition ability of Cry104-Cas12a/gRNA conjugate for the recognition of Crypto was evaluated based on the fluorescence signal of recognized Tex-Crypto. Briefly, 100 uL 10 ug/mL of Abs-Cas12a/gRNA conjugates were added into the 9018 high binding plate and incubated at RT for 1 hour. In the meanwhile, 100 uL 10 ug/mL pristine Cry104 antibody was added into the 9018 high binding plate for the same procedure for comparison. After the incubation, 350 μL PBS buffer was added into each well to remove the free biomolecules. Then, 350 μL incubation buffer was added into each well and incubated for 1 hour at room temperature for blocking. The incubation buffer was removed and various concentrations of Tex-Crypto were added into the high binding plate (100000, 50000, 25000, 12500, 0) for 1 hour incubation. The unbound Tex-Crypto was washed away using 350 μL wash buffer. Finally, the fluorescence signal was tested using ID3 plate reader with excitation wavelength of 570 nm and emission wavelength of 615 nm.

### The Detection of Single Crypto Using Cry104-Cas12a/gRNA Conjugate Based CRISPR/Cas12a System

First, 100 μL of 10 μg/mL streptavidin PBS solution was added into the wells of 9018 high-binding plate, and incubated at room temperature for one hour. Plates were then washed using 350 μL PBS buffer, twice.

Secondly, 350 μL incubation buffer was added into each well and incubated at room temperature for one hour before removal of the incubation buffer.

Thirdly, 100 μL of 10 μg/mL biotinylated Cry-104 antibody was added into each well at room temperature for one hour to form the sensing interface. Then we washed the plate using 350 μL EasyStain wash buffer, twice.

Fourth step was to add 200 μL Crypto samples with different numbers (0, 1, 2, 5) into each well and incubate for 1 hour at room temperature, then wash with 350 μL EasyStain wash buffer, twice.

Fifth step was to add 100 μL 10 μg/mL Cry104-Cas12a/gRNA conjugate into each well at room temperature for one hour. Then wash the plate using 350 μL PBS buffer.

Sixth step was to add 100 μL CRISPR/Cas12a reporter solution into each well, and then add 5 μL 1 μM triggering ssDNA into each well. The final mixture was incubated for 120 min at room temperature.

Finally, the fluorescence signal was tested using an iD3 plate reader with excitation wavelength of 570 nm and emission wavelength of 615 nm.

### Results

### Synthesis and Evaluation of Trans-Cleavage Ability of Cry104-Cas12a/gRNA Conjugate

The synthesis of Cry104-Cas12a/gRNA conjugate (or bi-functional antibody Cas12a/gRNA (BAC) conjugate) was performed using a commercial conjugation kit and detailed procedures were listed in Materials and Methodology point 2 above. For comparison, Cry104-Cas12a was prepared using the same procedure, and gRNA was not added in the synthesis step.

A schematic of the formation of the BAC is shown in FIG. 19a panel A. The formation of the BAC conjugate was investigated by using size distribution determined by the Zeta-sizer. As shown in FIG. 19a panel B), the peak of size distribution of Cas/gRNA was 6.1+/−0.1 nm, and the peak size of the antibody was 7.1+/−0.1 nm. After the formation of the conjugate, the peak size distribution of the conjugate was found to be 13.1+/−0.1 nm. This closely corresponds with the expected size increase when two protein molecules are conjugated, confirming a successful synthesis of the conjugate. Additionally, EMSA has been used to verify the successful formation of the BAC conjugate. As shown in FIG. 19a panel C), as the Cas12a nuclease (˜156 kDa) has a similar molecular weight to a typical antibody (IgG, ˜150 kDa), hence they have shown a similar movement pattern in gel electrophoresis. However, the significantly delayed movement pattern on the gel for the putative BAC conjugate indicated its successful formation, as the conjugation of both proteins led to a significantly increased total molecular weight.

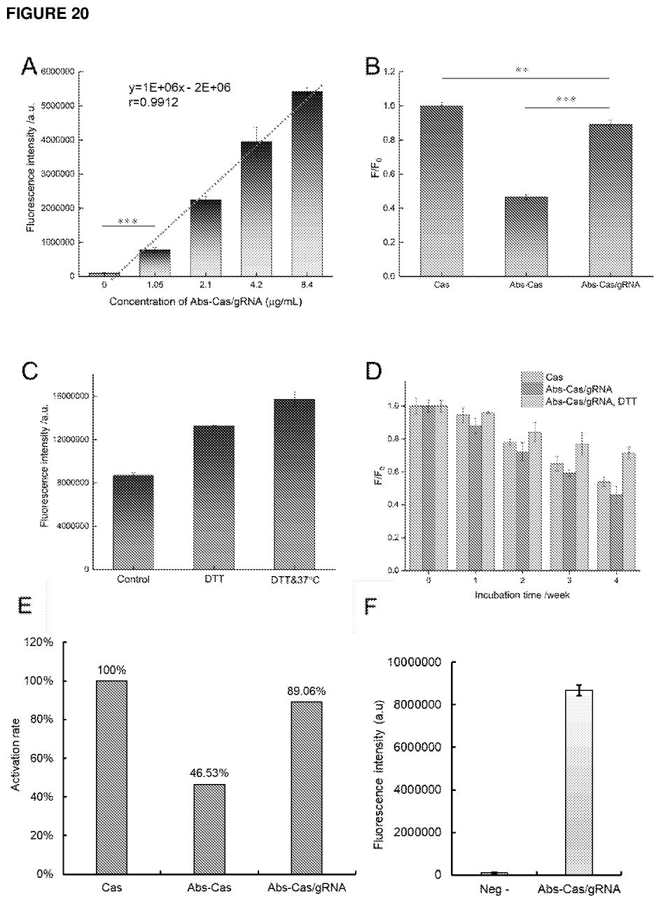

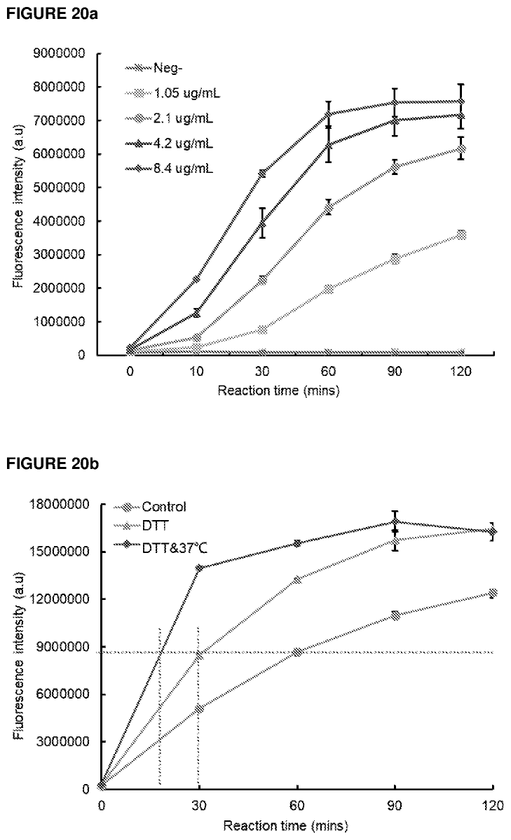

After the formation of BAC conjugate, its trans-cleavage of ssDNA was investigated. The intact antibody was used as a negative control, as well as different concentrations of BAC conjugate and the CRISPR reaction solution with 1 μM of triggering ssDNA. The fluorescence signals were tested after 30 min incubation. The result show that all concentrations of the BAC conjugate, from 1.05 μg/mL to 8.4 μg/mL, generated a significantly higher fluorescence signals compared with negative control with no BAC conjugate (FIG. 20, panel A), FIG. 20a), indicating that the BAC conjugate retains its original Cas12a-based trans-cleavage activity for signal generation and amplification. More importantly, the observed a high degree of linear correlation (R=0.9912) between the concentration of conjugate and the fluorescence signal. This indicates that the fluorescence signal generated by the BAC conjugate—can be used for quantitative detection of target analytes (FIG. 20, panel A)).

In addition, the change of trans-cleavage efficiency of Cas12a forming part of the BAC conjugate was evaluated. To this end, Cas12a protein was conjugated with the anti-Crypto antibody to create the BAC conjugates, with two different approaches, where Cas12a was either loaded with its gRNA before or after conjugation with the anti-Crypto antibody. Trans-cleavage of the Cas12a in both BAC conjugates thus prepared was then evaluated, in comparison to unconjugated Cas12a protein. Both conjugated Cas12a demonstrated the expected trans-cleavage function (FIG. 20, panel B), panel E)). However, loading gRNA to Cas12a protein after the conjugation process led to the loss of Cas12a nuclease activity by more than 50%, while preparing Cas12a/gRNA RNP before conjugation to antibody was able to prevent such loss and retain of ˜90% Cas12a activity. Hence, the trans-cleavage efficiency of the BAC conjugate is significantly higher when using Cas12a/gRNA RNP instead of a Cas12a protein not pre-loaded with gRNA in the conjugation process.

Overall, the application of the BAC conjugate loaded with its gRNA before conjugation produced >80 times increase in fluorescent intensity for signal amplification compared with control reaction with antibody only and without BAC conjugate (FIG. 20, panel F)).

In order to further enhance the trans-cleavage activity of the BAC conjugate, the effects of chemical enhancers and temperature were also investigated. The inventors found that with the addition of DTT (10 mM), the fluorescence signal increased ˜1.4 times. In addition, combining the use of DTT with the increase of reaction temperature from room temperature (25° C.) to 37° C. can further lift the fluorescence signal output by a factor of approximately 2 in comparison to the control reaction without the use of DTT and 37° C. (FIG. 20, panel C)). More importantly, this result also indicates that the reaction time to reach a designated final fluorescence intensity level can be reduced by up to 70% by adjusting these two factors (FIG. 20b). The potential interference of EasyStain buffers has been tested as well, and no significant negative effects have been observed on the Cas12a trans-cleavage activity. Furthermore, the stability of BAC conjugate's nuclease activity was also investigated. To this end, the prepared BAC conjugates with or without adding DTT, were stored in the reaction buffer at 4° C. for four weeks, and their trans-cleavage was compared to that in an unconjugated Cas12a protein. For the unconjugated Cas12a, its trans-cleavage efficiency was found to be reduced to ˜53.9% after four weeks, which is similar to the BAC conjugate (˜trans-cleavage efficiency reduced to ˜46.1%) (FIG. 20, panel D)). In the same conditions, the BAC conjugate with added DTT has shown a significantly higher fluorescent signal, indicative of ˜71.4% enzymatic activity still remaining. Therefore, it has been identified that DTT is both an effective trans-cleavage enhancer and also acts as a protective agent supporting the stability of BAC conjugate during 4° C. storage.

### Evaluation of the Recognition Ability of Cry104-Cas12a/gRNA Conjugate

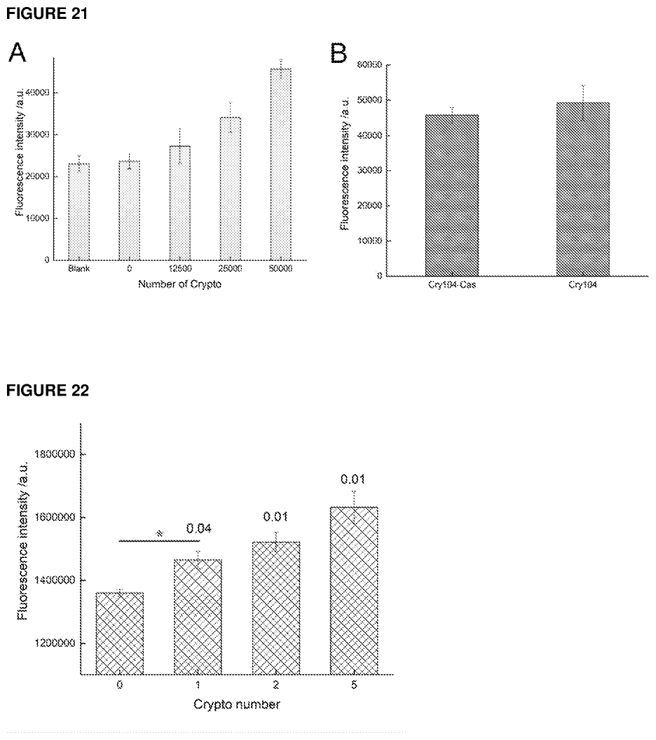

In order to evaluate the ability of Cry104-Cas12a/gRNA conjugate to recognize and bind Crypto, a plate based method was applied. The Cry104-Cas12a/gRNA conjugate was first immobilized on the high binding plate with following blocking, then various concentrations of Tex-Crypto were applied. As shown in FIG. 21A, the fluorescence signal increased with the increase of Crypto concentration, indicating the feasible target recognition and binding ability of the Cry104-Cas12a/gRNA conjugate. Further comparison was evaluated as shown in FIG. 21B. Compatible fluorescence signals of Cry104-Cas12a/gRNA conjugate and Cry104 were observed, indicating the compatible performance of Cry104-Cas12a/gRNA conjugate with Cry104.

### Application of Cry104-Cas12a/gRNA Conjugate for the Detection of Cryptosporidium

As shown in FIG. 22, various Crypto samples with exact number (0, 1, 2, 5 in 200 uL saline sample) were applied for the evaluation of the Cry104-Cas12a/gRNA conjugate based CRISPR/Cas12a biosensing system. After two hours incubation of the CRISPR/Cas solution, significant fluorescence difference was observed. The fluorescence signal linearly increased with the increase of Crypto numbers, and the calculated P value between single Crypto and 0 was 0.04, which indicates that the Cry104-Cas12a/gRNA conjugate based CRISPR/Cas biosensing system is able to detect single Crypto.

### Conclusions

In this research, the inventors have demonstrated for the first time the successful development of an Antibody-CRISPR/Cas effector protein conjugate (Cry104-Cas12a/gRNA conjugate) which can be applied for the detection of an analyte (e.g. a low abundance analyte). In this particular example for the sensitive detection of single microorganism has been exemplified. The synthesis of the Cry104-Cas12a/gRNA conjugate described herein represents the first demonstration of the use of a chemical conjugation method for the conjugation of any antibody with a Cas protein. The trans-cleavage ability of Cry104-Cas12a/gRNA conjugate was first evaluated, and it was surprisingly determined that performance was markedly increased where the Cas protein was conjugated in combination with gRNA. Furthermore, it was surprisingly found that the trans-cleavage ability of the Ab-Cas12a/gRNA conjugate was further enhanced by adding DTT in the reaction buffer. In addition, the recognition ability of Cry104-Cas12a/gRNA conjugate was demonstrated and evaluated, and compatible performance was observed. Finally, the optimized Cry104-Cas12a/gRNA conjugate based CRISPR/Cas12a biosensing system was successfully applied for the detection of signal Crypto. This is a significantly improved performance comparing to common commercial immunoassay kits for Crypto, which generally only produce Positive/Negative results for the presence of high concentration of Crypto. The ultralow sensitivity of the CRISPR-powered BAC conjugate immunoassay represents a significant step forward for waterborne pathogen surveillance, where the required detection limits is beyond the capability of conventional immunoassays. Therefore, the conjugation method of antibody with Cas protein provides an effective approach for the development of CRISPR/Cas based biosensing system for the measurement of diverse targets including microorganisms, small molecules and small proteins.

### Example 5. Enhancement of Trans-Cleavage of Cas12a and Cas13a with Chemical Enhancer Enables Amplified Nucleic Acid Detection

### Materials and Methodology

### 1. Materials and Reagents

Bioreagents: EnGen® Lba Cas12a (Cpf1) protein (New England Biolab), 10×NEB 2.1 buffer (New England Biolab), Agarose tablet (Meridian Bioscience, BIO-41027), 10 bp DNA Ladder (NEB, N0558S), 6× loading dye (NEB, B7024S), 1× buffer TBE. 10,000× Gel Red (Biotium, 41003), QuantiNova SYBR Green RT-PCR kit (Qiagen, 208352), DNase/RNase free water (ThermoFisher), phosphate buffered saline (PBS) (Sigma, 10 mM, pH=7.4), E. coli (pC013-Twinstrep-SUMO-huLwCas13a, Addgene), Tryptone (Thermo Fisher Scientific, Cat No. LP0042T), yeast extract (Thermo Fisher Scientific, Cat No. 212750),

Chemicals: Dithiothreitol (DTT) (Sigma, DTT-RO), Tris(2-carboxyethyl)phosphine (TCEP) (Sigma, C4706), 2-Mercaptoethanol (2-ME) (Sigma, 63689), Tween 20 (Sigma, T2700), Triton X-100 (Sigma, X100), Brij L23 (Sigma, B4184), Sodium deoxycholate (NaDC) (Sigma, D6750), Sodium dodecyl sulfate (SDS) (Sigma, L3771), Hexadecyltrimethylammonium bromide (CTAB) (Sigma, H5882), Poly(ethylene glycol) (PEG) (Sigma, P5413), Poly(vinyl alcohol) (PVA) (Sigma, P8136, 363138), KH₂PO₄ (Thermo Fisher Scientific, Cat No. AJA391-500), K₂HPO₄ (Thermo Fisher Scientific, Cat No. AJA621-500), glycerol (Chem-supply, Cat No. GA010), Na₂HPO₄ (Thermo Fisher Scientific, Cat No. AJA621-500), NaH₂PO₄ (Thermo Fisher Scientific, Cat No. AJA471-500), NaCl (Thermo Fisher Scientific, Cat No. AJA465-5), imidazole (Astral Scientific, Cat No. BIOIB0277-500), MgCl₂ (Thermo Fisher Scientific, Cat No. AJA296-500), CaCl₂ (Thermo Fisher Scientific, Cat No. AJA127-500), Tris-HCl (Sigma, Cat No. T3253), HEPES (Sigma, Cat No. H3375), DTT (PanReac AppliChem, Cat No. A29480025), protease inhibitors (Sigma, Cat No. 4693132001) DNase (Sigma, Cat No. D5025-150KU), 5 mL HisTrap column (QIAGEN, Cat No. 30761), HiLoad 16/60 Superdex 200 (GE Healthcare Life Sciences, Cat No. 17-1069-01), Nuclease-Free water (Sigma, Cat No. W4502),

Oligos: All designed RNA and DNA oligos were synthesized by Sango Biotech. For the Texas Red reporter based CRISPR/Cas biosensing system, the excitation wavelength was 570 nm and emission wavelength was 615 nm. For the Cy5.5 reporter based CRISPR/Cas biosensing system, the excitation wavelength was 680 nm and emission wavelength was 720 nm. For the FAM reporter based CRISPR/Cas biosensing system, the excitation wavelength was 480 nm and emission wavelength was 520 nm.

TABLE 4
RNA and DNA oligo sequences used in CRISPR/Cas12a
system for the optimization and evaluation
of the performance of chemical activators.

| | Oligo | | Length | Modifi- |
|---|---|---|---|---|
| Name | type | Sequence 5′-3′ | (nt) | cation |
| Guiding | ssRNA | UAA UUU CUA | 42 | N/A |
| RNA | | CUA AGU GUA | | |
| (gRNA) | | GAU GGG GGG | | |
| | | GGU UGG UAG | | |
| | | GGU GUC | | |
| | | (SEQ ID No. 6) | | |
| Triggering | ssDNA | GAA GAC ACC | 24 | N/A |
| ssDNA | | CTA CCA ACC | | |
| | | CCC CCC | | |
| | | (SEQ ID No. 5) | | |
| ssDNA | ssDNA | TTATT | 5 | 5′-Texas |
| target 1 | | | | Red; |
| | | | | 3′-BHQ2 |
| ssDNA | ssDNA | TTATT | 5 | 5′-Cy5.5; |
| target 2 | | | | 3′-BHQ2 |
| ssRNA | ssRNA | UUAUU | 5 | 5′-FAM; |
| target | | | | 3′-BHQ1 |

TABLE 5
RNA and DNA oligos sequences used in CRISPR/
Cas12a system for the detection of HP gene.

| | Oligo | | Length | Modifi- |
|---|---|---|---|---|
| Name | type | Sequence 5′-3′ | (nt) | cation |
| HP 16s FW | ssDNA | TCG TTT AGG | 20 | N/A |
| primer | | GCG TGG ACT | | |
| | | AC | | |
| | | (SEQ ID No. 7) | | |
| HP 16s RV | ssDNA | GGC GAC CTG | 20 | N/A |
| primer | | CTG GAA CAT | | |
| | | TA | | |
| | | (SEQ ID No. 8) | | |
| HP 16s | ssRNA | UAA UUU CUA | 45 | N/A |
| gRNA | | CUA AGU GUA | | |
| | | GAU GCG CAA | | |
| | | UCA GCG UCA | | |
| | | GUA AUG UUC | | |
| | | (SEQ ID No. 9) | | |
| ssDNA | ssDNA | TTATT | 5 | 5′-Texas |
| reporter | | | | Red; |
| | | | | 3′-BHQ2 |

TABLE 6
RNA and DNA oligos sequences used in CRISPR/
Cas12a system for the detection of SARS-Cov-2 gene.

| | Oligo | | Length | Modifi- |
|---|---|---|---|---|
| Name | type | Sequence 5′-3′ | (nt) | cation |
| CoV-N FW | ssDNA | CAGAATGGAGAACGC | 19 | N/A |
| primer | | AGTG | | |
| | | (SEQ ID No. 10) | | |
| CoV-N RV | ssDNA | GTGAACCAAGACGCA | 20 | N/A |
| primer | | GTATT | | |
| | | (SEQ ID No. 11) | | |
| Cas12a- | ssRNA | UAA UUU CUA CUA | 42 | N/A |
| CoV-N | | AGU GUA GAU CCC | | |
| gRNA | | AAU AAU ACU GCG | | |
| | | UCU UGG | | |
| | | (SEQ ID No. 12) | | |
| ssDNA | ssDNA | TTATT | 5 | 5′-Texas |
| reporter | | | | Red; |
| | | | | 3′-BHQ2 |

TABLE 7
RNA oligo sequences used in CRISPR/Cas13a
system for the optimization and evaluation
of the performance of chemical activators.

| | Oligo | | Length | Modifi- |
|---|---|---|---|---|
| Name | type | Sequence 5′-3′ | (nt) | cation |
| Guiding | ssRNA | GGA CCA CCC CAA | 50 | N/A |
| RNA | | AAA CGA AGG GGA | | |
| (gRNA) | | CUA AAA CAG AGA | | |
| | | AUU CCA UAG UCC | | |
| | | AG | | |
| | | (SEQ ID No. 13) | | |
| Triggering | ssRNA | GGA CUG GAC UAU | 32 | N/A |
| ssRNA | | GGA AUU CUC GGG | | |
| | | UGC CAA GG | | |
| | | (SEQ ID No. 14) | | |
| ssRNA | ssRNA | UUUUU | 5 | 5′-FAM; |
| target | | | | 3′-BHQ1 |

TABLE 8
RNA oligo sequences used in CRISPR/Cas13a
system for the detection of SARS-Cov-2 gene.

| | Oligo | | Length | Modifi- |
|---|---|---|---|---|
| Name | type | Sequence 5′-3′ | (nt) | cation |
| Cas13a- | ssRNA | GGG GAU UUA GAC | 60 | N/A |
| CoV-N | | UAC CCC AAA AAC | | |
| gRNA | | GAA GGG GAC UAA | | |
| | | AAC ACGU UGUUU | | |
| | | UGAUC GCGCC CC | | |
| | | (SEQ ID No. 15) | | |
| CoV-N | ssRNA | CAGAA UGGAG AACGC | 80 | N/A |
| target | | AGUGG GGCGC GAUCA | | |
| | | AAACA ACGUC GGCCC | | |
| | | CAAGG UUUAC CCAAU | | |
| | | AAUAC UGCGU CUUGG | | |
| | | UUCAC | | |
| | | (SEQ ID No. 16) | | |
| ssRNA | ssRNA | UUUUU | 5 | 5′-FAM; |
| reporter | | | | 3′-BHQ1 |

### 2. Reverse Transcription Polymerase Chain Reaction (RT-PCR) Amplification of SARS-CoV-2 Gene Used in the Optimized CRISPR/Cas 12a Biosensing Assay.

The 20 μM (520 ng/mL) synthesized SARS-CoV-2 RNA gene fragment has been 10 times series diluted in PBS buffer for one-step RT-PCR. Then, the QuantiNova SYBR Green RT-PCR kit was used for each RT-PCR reaction: 10 μL 2×RT-PCR master mix, 0.2 μL QN RT mix, 7.3 μL RNase free water, 1 μL 10 μM of each primer, and adding with 0.5 μL each concentration of the diluted SARS-CoV-2 RNA gene sample. The reaction was set at condition as: 50° C. 10 mins, 95° C. 2 mins, and 35 cycles for 95° C. 8 sec, 60° C. 15 sec.

### 3. Purification of LwCas13a Protein

The LwCas13a protein was produced and purified as described¹. Briefly, 10 mL overnight culture in Terrific Broth medium (12 g/L tryptone, 24 g/L yeast extract, 2.3 g/L KH₂PO₄, 12.54 g/L k₂HPO₄, 4 mL/L glycerol) (TB) was used to inoculate 1 L of TB for growth at 37° C. and 180 RPM. When OD600 achieved 0.6, the LwCas13a expression was induced with IPTG to a final concentration of 500 μM. The cells were then cultured at 18° C. for 18 h for protein production. Cells were then harvested and resuspended in the lysis buffer (50 mM sodium phosphate, 300 mM NaCl, 20 mM imidazole, pH 8.0) supplemented with protease inhibitors, MgCl₂/CaCl₂ and DNase. The cells were disrupted through sonication. The cell lysate was removed by centrifugation for 30 minutes at 4° C. and 10,000 g. The supernatant was filtered through 0.45 μm filters and then 0.22 μm filters. Protein was loaded onto a 5 mL HisTrap column via FPLC with binding buffer (50 mM sodium phosphate, 300 mM NaCl, 20 mM imidazole, pH 8.0) and eluted with elution buffer (50 mM sodium phosphate, 300 mM NaCl, 500 mM imidazole, pH 8.0). The eluted fractions were tested for presence of LwCas13a by SDS-PAGE. The fractions containing LwCas13a were pooled and concentrated via a Centrifugal Filter Unit to 5 mL. The concentrated protein was loaded onto a HiLoad 16/60 Superdex 200 column via FPLC with running buffer (10 mM HEPES, 1 M NaCl, 5 mM MgCl₂, 2 mM DTT, pH 7.0). The components of eluted fractions from size exclusion chromatography were characterized by SDS-PAGE. The fractions containing LwCas13a were pooled and concentrated into storage buffer (600 mM NaCl, 50 mM Tris-HCl, 15% glycerol, 2 mM DTT, pH 7.5) and frozen at −40° C. for further use.

### 4. Preparation of Reaction Buffer for CRISPR/Cas13a Biosensing System

Briefly, the high concentration storage solutions for each component (1 M Tris-HCl, 5 M NaCl, and 1 M MgCl₂) were prepared in Nuclease-Free Water and filtered through 0.22 μm filters. Then, the 10× Reaction Buffer (400 mM Tris-HCl, 600 mM NaCl, 60 mM MgCl₂, pH 7.3) was prepared and stored in −20° C. for further use.

### 5. Standard Procedure for the Evaluation of Trans-Cleavage Activity of Cas12a

For all trans-cleavage activity evaluation experiments, the CRISPR/Cas12a reaction mixture was prepared as follows: 1 μL 100 μM (100 μmol) of Cas12a protein was gently mixed with 5 μL 20 μM (100 μmol) gRNA in 3.6 mL 1×NEB 2.1 buffer. Then, 6 μL 100 μM (0.6 nmol) of pre-synthesized ssDNA or ssRNA targets were added and well mixed to form the standard reaction mixture. Different chemical enhancers (sulfhydryl reductant and surfactant) were first dissolved in MilliQ water to produce high concentration storage solutions, then a certain amount of storage solution was added into the standard CRISPR/Cas12a reaction mixture to realize the final reaction mixture with defined concentration of enhancers. All the relevant DNA and RNA oligo sequences are listed in Table 4.

5 μL 1 μM single strand trigger DNA was applied in 100 μL prepared CRISPR/Cas12a reaction mixture for starting the CRISPR reaction process. SpectraMax i3x multi-Mode Microplate Reader (Molecular devices) was applied for the detection of fluorescence readout.

### 6. Electrophoretic Mobility Shift Assay for Verifying the Performance of Chemical Enhancers on the CRISPR/Cas12a Biosensing System

The electrophoretic mobility shift assay (EMSA) was also applied to evaluate the trans-cleavage performance of the CRISPR/Cas12a system in a way that does not depend on the fluorescent sensing signal. This is because fluorescence process may be independently affected by chemical enhancers used here. Briefly, the standard CRISPR/Cas12a reaction mixture was prepared as per point 1 above, then chemical enhancers were added at concentrations determined to be optimal (10 mM DTT, 500 μM TCEP, 1% PVA, 0.04% Brij23), and the same volume of 1×PBS was also used as the control. Then, 50 μL of CRISPR/Cas12a and CRISPR/Cas12a enhancer mixture was mixed with 4 μL 10 μM trans-revealing ssDNA, and 1 μL of triggering ssDNA at different concentrations (0, 1 μM, 2 μM, 5 μM, 10 μM). Alternatively, 50 μL of CRISPR/Cas12a and the chemical enhancer mixture was firstly mixed with 1 μL different concentrations of target ssDNA (5 μM, 10 μM, 15 μM, 20 μM), and then either 1 μL 1 μM triggering ssDNA was added or 1 μL PBS as control. The reaction mixtures were incubated at 26° C. for 60 mins, and then deactivated at 65° C. for 15 mins. Afterwards, 10 μL of each deactivated reaction mixture was mixed with 2 μL 6× loading dye and loaded onto 3% Agarose gel with 1× GelRed nucleic acid dye for voltage-constant electrophoresis at 60V for 100 mins.

7. Application of Optimized CRISPR/Cas12a Biosensing System for the Detection of Helicobacter pylori (HP) Gene

The optimized CRISPR/Cas12a reaction mixtures for the detection of Helicobacter pylori (HP) gene (16S) were prepared as follows: 360 μL 100 mg/mL (36 mg) PVA, 36 μL 1M (36 μmol) DTT, and 360 μL 10 times concentrated NEB 2.1 buffer were mixed together, then add MilliQ water to the mixed solution until 3.6 mL. Subsequently, 1 μL 100 μM (100 μmol) of Cas12a protein, 5 μL 20 μM (100 μmol) HP gRNA, and 6 μL 100 μM (0.6 nmol) of pre-synthesized ssDNA linked fluorescent reporter (Texas Red) were added in the solution and mixed well to form the optimized reaction mixture. All the relevant DNA and RNA oligo sequences are listed in Table 5.

5 μL of different concentrations of double strand target genes generated by PCR (0, 12, 49, 195, 780, 3125, and 12500 pM) were added to 100 μL of the prepared CRISPR/Cas12a reaction mixture for the CRISPR reaction process.

### 8. Application of Optimized CRISPR/Cas12a Biosensing System for the Detection of SARS-CoV-2 Gene

The optimized CRISPR/Cas12a reaction mixtures for the detection of SARS-CoV-2 gene were prepared as follows: 360 μL 100 mg/mL (36 mg) PVA, 36 μL 1 M (36 μmol) DTT, and 360 μL 10 times concentrated NEB 2.1 buffer were mixed together, then add MilliQ water to the mixed solution until 3.6 mL. Subsequently, 1 μL 100 μM (100 μmol) of Cas12a protein, 5 μL 20 μM (100 μmol) SARS-CoV-2 gRNA, and 6 μL 100 μM (0.6 nmol) of pre-synthesized ssDNA linked fluorescent reporter (Texas Red) were added in the solution and mixed well to form the optimized reaction mixture. All the relevant DNA and RNA oligo sequences are listed in Table 6.

5 μL of different concentrations of double strand target SARS-CoV-2 gene generated by RT-PCR (0, 0.78, 3.1, 12.5, 50, 100, and 200 fM) were applied in 100 μL prepared CRISPR/Cas12a reaction mixture for starting the CRISPR reaction process.

### 9. Standard Procedure for the Evaluation of Trans-Cleavage Activity of Chemically Induced CRISPR/Cas13a Biosensing System

For all trans-cleavage evaluation experiments, the CRISPR/Cas13a reaction mixture was prepared as follows: 10 μL 4 μM (40 μmol) of Cas13a protein was gently mixed with 2 μL 20 μM (40 μmol) gRNA in 1 mL reaction buffer. Then, 6 μL 20 μM (120 μmol) of pre-synthesized ssRNA target (FAM) was added and well mixed to form the standard reaction mixture. All the relevant DNA and RNA oligo sequences are listed in Table 7.

Diverse types of chemicals were first dissolved in MilliQ water to produce high concentration storage solutions, then a certain amount of storage solution was added into the standard CRISPR/Cas13a reaction mixture to realize the certain concentration of chemical involved final reaction mixture.

2 μL 1 μM single strand trigger RNA was applied in 100 μL prepared CRISPR/Cas13a reaction mixture for starting the CRISPR reaction process. ID3 plate reader was applied for the detection of fluorescence readout from FAM modified target with the excitation wavelength of 480 nm and emission wavelength of 520 nm.

### 10. Application of Optimized CRISPR/Cas13a Biosensing System for the Detection of SARS-CoV-2 Gene

The optimized CRISPR/Cas13a reaction mixtures for the detection of SARS-CoV-2 gene were prepared as follows: 10 μL 4 μM (40 μmol) of Cas13a protein was gently mixed with 2 μL 20 μM (40 μmol) gRNA in 1 mL reaction buffer. Then, 6 μL 20 μM (120 μmol) of pre-synthesized ssRNA linked fluorescent reporter (FAM) was added and well mixed to form the standard reaction mixture. Afterwards, 5 μL 100 mg/mL (0.5 mg) PVA, and 5 μL 1 M (5 μM) DTT were added into the mixture to form the optimized reaction mixture. All the relevant DNA and RNA oligo sequences are listed in Table 8.

5 μL of different concentrations of target genes (0, 195, 780, 3125 and 12500 pM) were applied in 100 μL prepared CRISPR/Cas13a reaction mixture for starting the CRISPR reaction process.

### Results

### 1. Sulfhydryl Reductant-Induced Enhancement of Trans-Cleavage in Cas12a

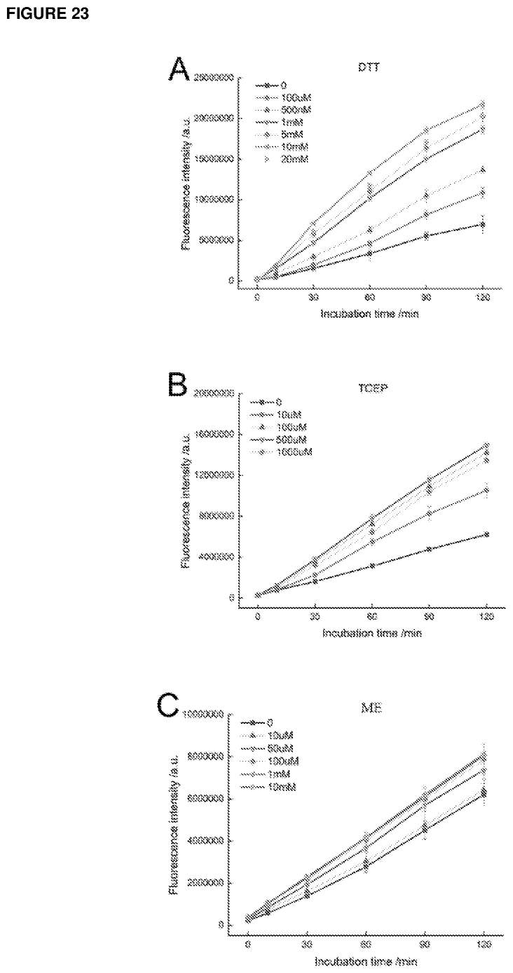

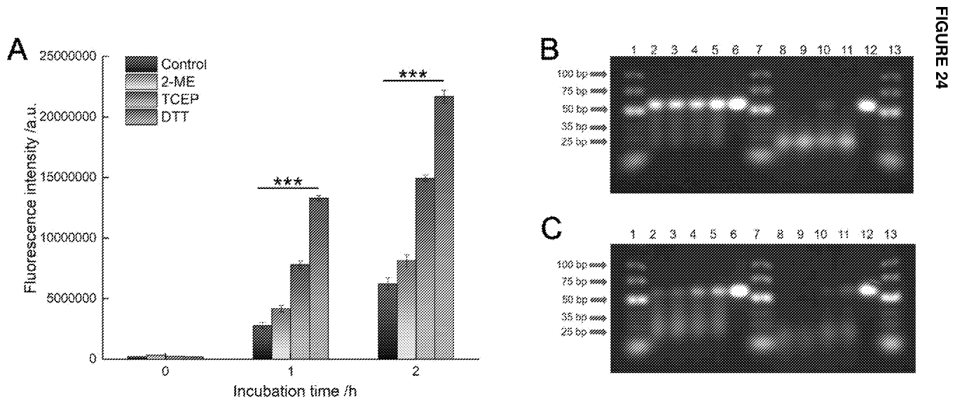

The inventors explored the effect of conventional sulfhydryl reductants on the trans-cleavage activity of Cas12a by adding three common sulfhydryl reductants (Dithiothreitol (DTT), Tris(2-carboxyethyl) phosphine (TCEP), and 2-Mercaptoethanol (2-ME)) to the reaction buffer. All were enhancing the trans-cleavage and the optimum concentrations leading to the highest trans-cleavage rates for DTT, TCEP, and 2-ME were found to be 10 mM, 500 μM, and 1 mM, respectively (FIG. 23). In order to compare the trans-cleavage enhancement effect between these three sulfhydryl reductants, their optimised concentrations were applied, and the fluorescence signals obtained in the standard procedure were compared (FIG. 24A). It was found that at the same incubation time point, the sequence of fluorescence intensity was lowest in the control sample increasing for 2-ME, TCEP, and was highest for DTT.

Additionally, based on the actual fluorescence intensity values at the incubation time of 2 h, the fluorescence intensity values of 2-ME, TCEP, and DTT were 3.4, 2.4, 1.3 fold of that in the control, respectively. Therefore, DTT shows the best enhancement effect of Cas12a biosensing which could be due to trans-cleavage or modification (e.g. enhancement) of fluorescence by the chemical enhancer.

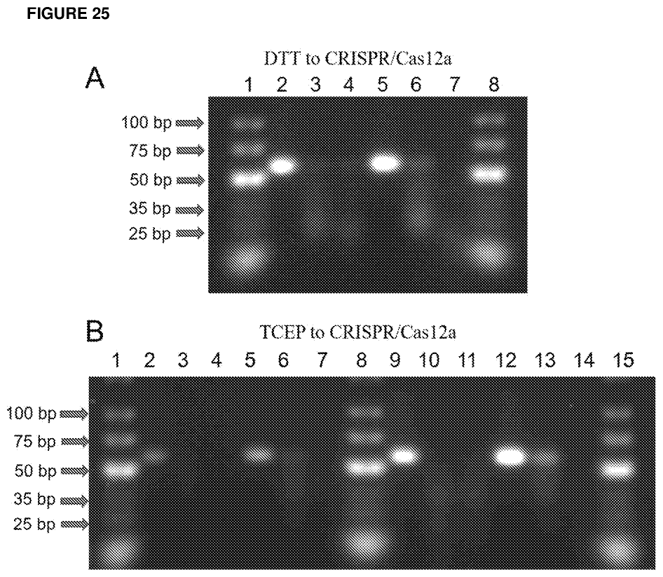

In order to validate that the observed enhancement effect is indeed due to trans-cleavage modification in a sulfhydryl reductant-assisted CRISPR/Cas12a biosensing system, the activator DTT was tested with Cas12a using an electrophoretic mobility shift assay (FIG. 24B). The results show that the brightness of strips at 50 bp increased between columns 2 and 6, which was due to the decrease of trigger ssDNA concentration from 10 μM to 0. After adding the optimized concentration of DTT (10 mM) to Cas12a, the corresponding columns from 8 to 11 at 50 bp were all shifted downwards, indicating an increased cleavage of the target DNA consistent with a strong enhancement effect of activator DTT. A similar effect was observed for TCEP (FIG. 24C). Further demonstration of the enhancement effects of DTT or TCEP in a complete CRISPR/Cas12 biosensing systems was investigated in FIG. 25, and similar enhancement effects were observed.

### 2. Non-Ionic Surfactant Induced Trans-Cleavage Enhancement for Cas12a

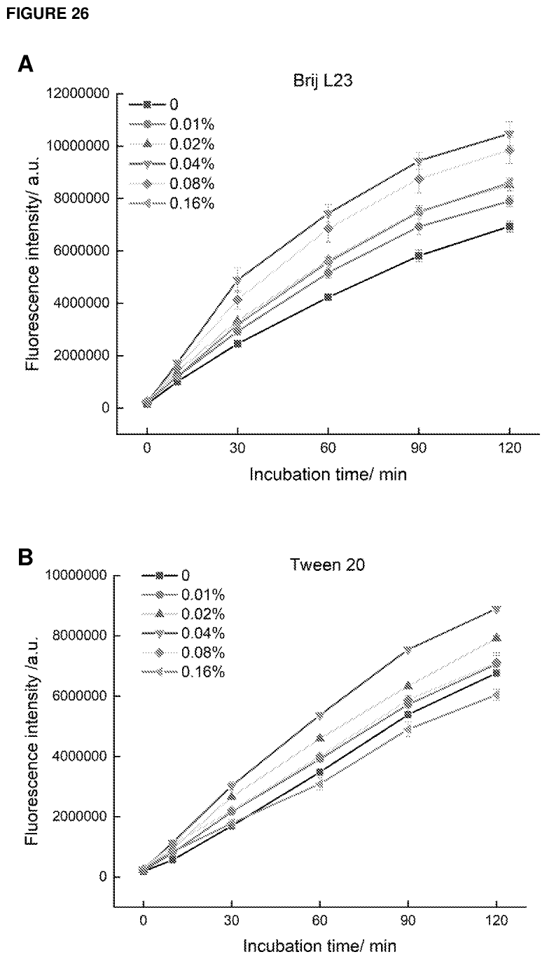

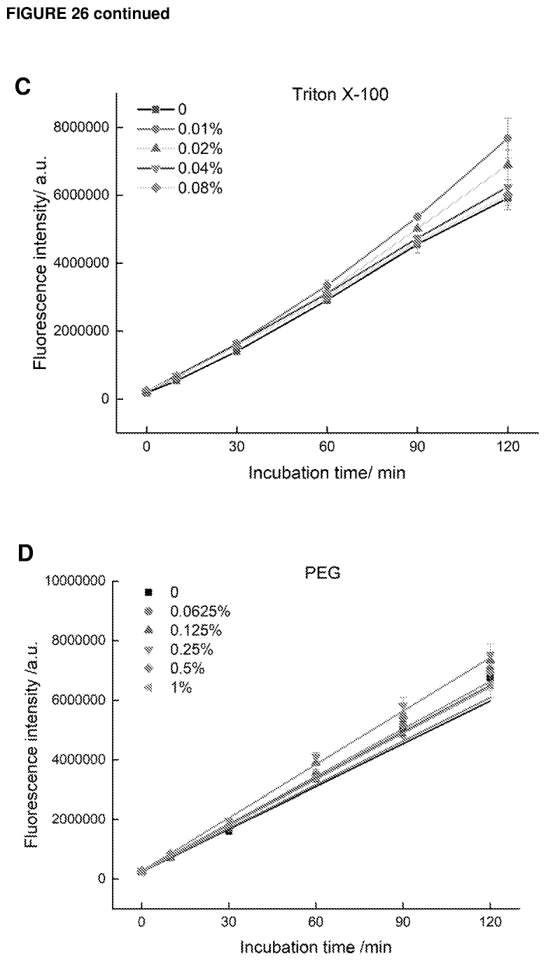

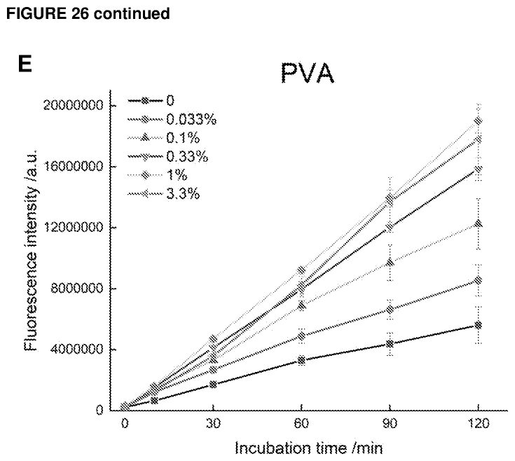

In this section, the impact of non-ionic surfactants on the trans-cleavage ability of Cas12a was investigated. Non-ionic polyol type surfactants Brij L23, Tween 20, Triton X-100, PEG and two types of polyol surfactant PVA-P8136 and PVA-363138 from Sigma were studied, and their optimum concentrations were found to be 0.04%, 0.04%, 0.01%, 0.25% and 1%, respectively (FIG. 26). The comparison of all the non-ionic surfactants was investigated based on their specific optimum concentrations. As shown in FIG. 27A, among the polyoxyethylene type non-ionic surfactants Brij L23 shows the best performance, with approximately a factor of two increase of the fluorescence signal compared with control, at the incubation time point of 120 min.

The investigation of polyol type non-ionic surfactant with Cas12a was conducted using common PVA surfactants. Two types of PVA (PVA-P8136 and PVA-363138 from Sigma) were investigated and enhanced Cas 12a trans-cleavage activity was observed with both (FIG. 27A). In addition, PVA-P8136 shows a stronger effect. Without being bound by any theory it is considered that this is related to higher molecular weight of PVA-P8136 (30,000-70, 000) compared to PVA-363138 (31,000-50, 000), which makes it a better stabilizer. The fluorescence intensity of our standard system with PVA-P8136 was approximately three times that of control at 120 min incubation.

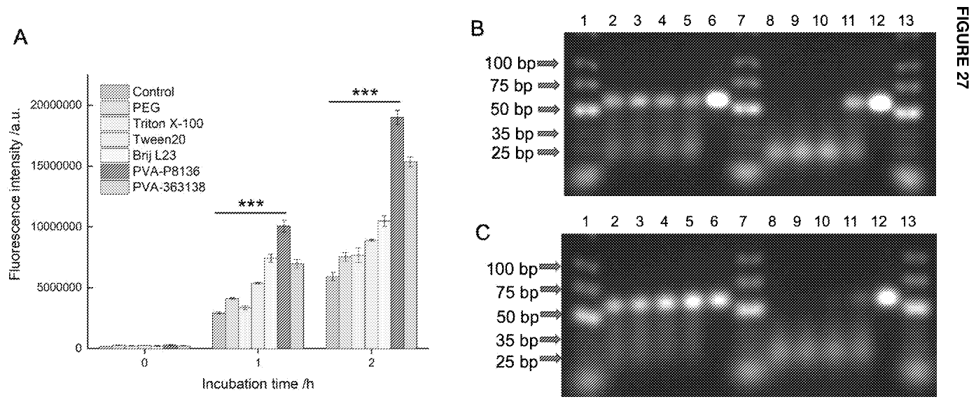

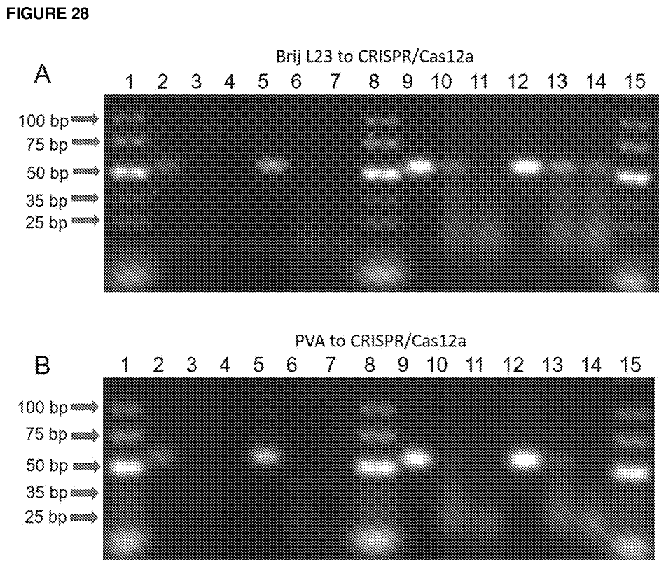

In order to validate that the observed enhancement effect is indeed due to trans-cleavage modification by non-ionic surfactant used with Cas12a, and not to some fluorescence enhancement/quenching effects, an electrophoretic mobility shift assay was applied. And Brij L23 (FIG. 27B) and PVA-P8136 (FIG. 27C) were selected as example non-ionic surfactants. As shown in FIG. 24B, the bands from column 8 to 11 shifted downwards with the addition of Brij L23, while the corresponding bands from 2 to 5 remain at around 50 bp in the absence of Brij L23, indicating the enhancement effect of non-ionic surfactant Brij L23 on the trans-cleavage activity of Cas12a. As shown in FIG. 24C, the bands present in columns 8 to 11 shifted to downwards with the addition of PVA compared to the corresponding columns from 2 to 5 without PVA, indicating the enhancement effect of PVA. Further demonstration of the enhancement effects of Brij L23 and PVA was conducted (FIG. 28), and similar enhancement effects were also observed.

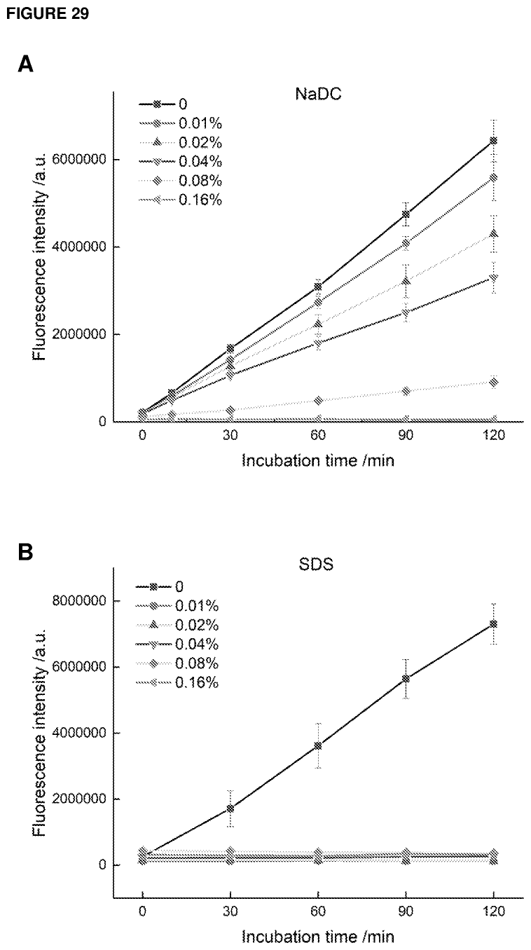

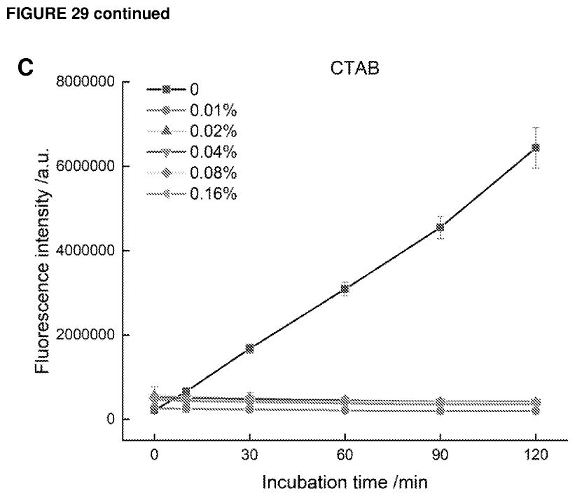

After investigation of the effects of non-ionic surfactant to the trans-cleavage ability of Cas12a, the effect of ionic (anionic and cationic) surfactants on the trans-cleavage ability of Cas12a was also tested. (FIG. 29). Negative impact was observed for all of the ionic surfactants test, which was possible due to the electrostatic interaction between the ionic surfactants and the CRISPR/Cas12a mixture.

### 3. Characterization of Chemically Enhanced Trans-Cleavage Ability of Cas12a

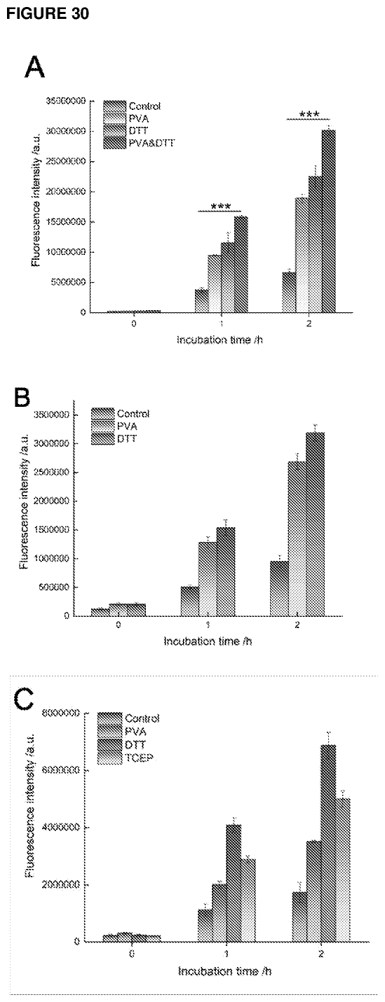

After demonstration of the enhancement effect of sulfhydryl reductants and non-ionic surfactants used with Cas12a, their combinations were compared (FIG. 30A). DTT and PVA were selected as the representative sulfhydryl reductant and non-ionic surfactant, respectively. As shown in FIG. 30A, DTT shows a higher enhancement effect than PVA. After combining of DTT with PVA, the fluorescence signal was further enhanced, demonstrating a synergistic 6 fold increase of the fluorescence signal of PVA and DTT alone after 120 min incubation. Therefore, PVA and DTT was the preferred combination, which was used for the following detection of the microbial nucleic acid target.

The effect of different fluorophores on target ssDNA of Cas12a was investigated as shown in FIG. 30B. As in all the previous experiments, Texas red-modified ssDNA target was applied, Cy5.5 was selected as an alternative to Texas Red on the ssDNA targets. Both fluorophores were then applied at the same concentration in the PVA or DTT-assisted CRISPR/Cas12a system (FIG. 30B). The fluorescence intensity value in the DTT-assisted Cas12a system in FIG. 30B for Cy5.5 was much lower than that of DTT-assisted Cas12a system with Texas red as shown in FIG. 30A. The same effect was also observed in the control and PVA-assisted Cas12a systems. The possible reason was due to the fluorescence efficiency of Cy5.5 was lower that of Texas red. However, the enhancement effect of both PVA and DTT were observed (FIG. 30B) and the enhanced fluorescence ratio in FIG. 25B was similar to that in FIG. 30A. For instance, the fluorescence signal of DTT assisted CRISPR/Cas12a biosensing system was around three times of that of controls without DTT at 120 min (FIG. 30B), which closely corresponds to the enhancement effect of DTT in the CRISPR/Cas12a biosensing system Texas with Texas Red-ssDNA target (FIG. 30A). Therefore, fluorophore differences on the ssDNA target have a negligible influence on the enhancement effect of chemical enhancers, and the enhancement performance was essentially due to the enhanced trans-cleavage ability of Cas12a effector to target ssDNA.

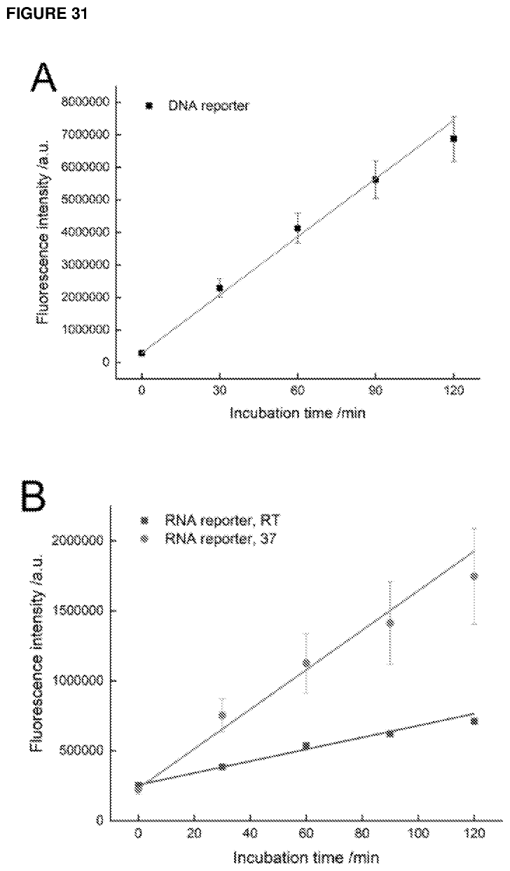

The specificity of target differences in a chemically enhanced Cas12a system was investigated. In addition to the trans-cleavage ability of Cas12a to target ssDNA, the trans-cleavage effect of ssRNA target of Cas12a is also observed. However, the trans-cleavage rate of ssRNA targets by Cas12a was found to be approximately 10-fold lower than the trans-cleavage rate of ssDNA targets (FIG. 31). In order to further enhance the trans-cleavage ability of CRISPR/Cas12a to ssRNA targets, sulfhydryl reductant (DTT and TCEP) and PVA were applied in the reaction buffer system with 37° C. incubation temperature, which was found to be optimal for the ssRNA target-based CRISPR/Cas12a biosensing systems. Enhanced performances of chemical enhancer-assisted CRISPR/Cas12 biosensing system were observed (FIG. 30C). Therefore, the enhancement effect of chemical enhancers to the trans-cleavage ability of Cas12a is effective both with ssDNA and ssRNA targets.

### 4. Chemically Enhanced CRISPR/Cas12a Biosensing System for the Detection of HP Gene and SARS-CoV-2 Gene.

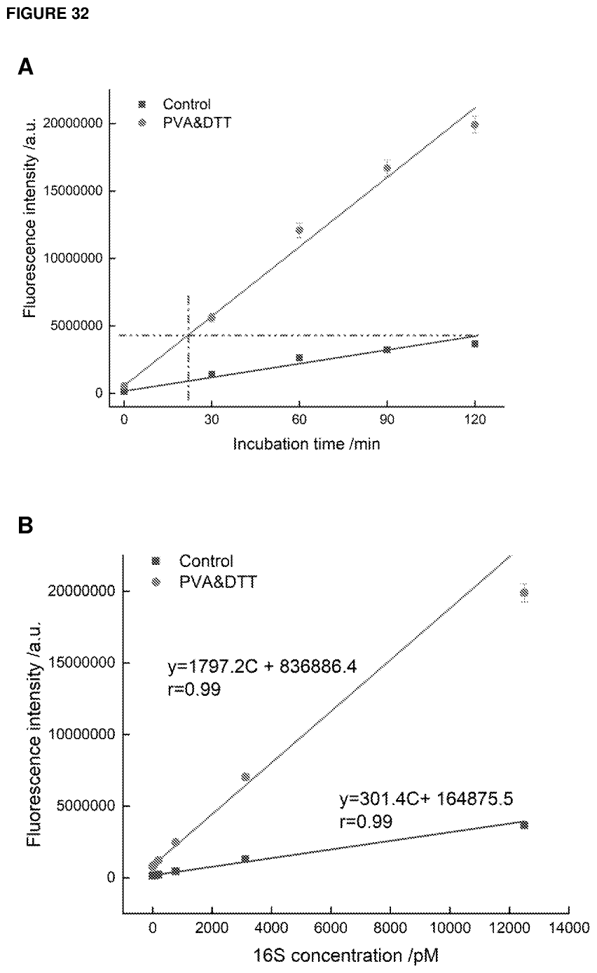

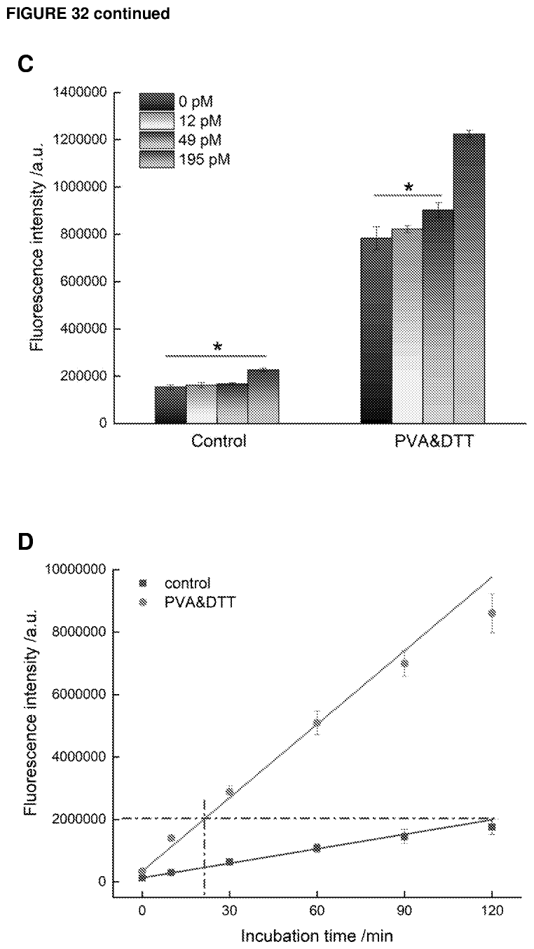

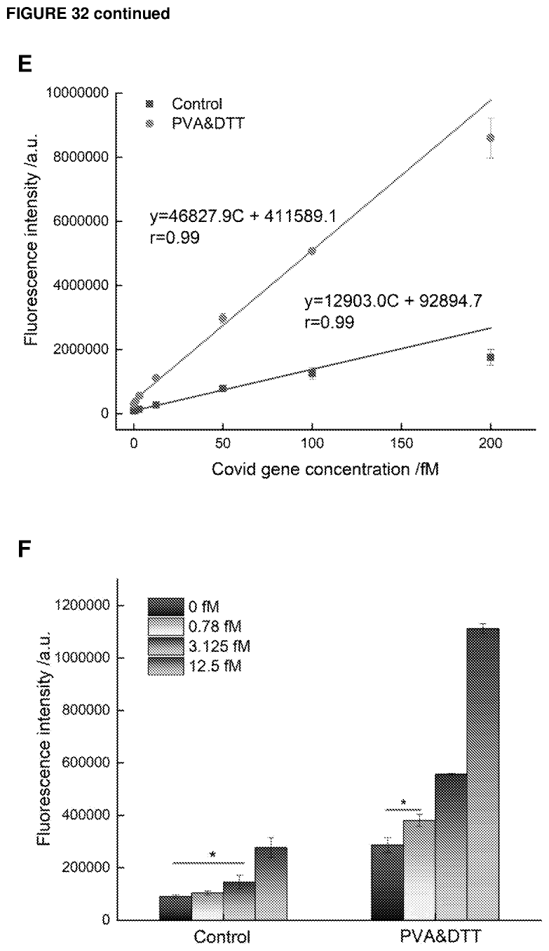

We applied the above findings to create an optimised original CRISPR/Cas12a biosensing system for the detection of pathogenic bacteria HP gene (16S). In this system, PVA+DTT assisted CRISPR/Cas12a biosensing system was applied with Texas red-modified ssDNA reporters. The CRISPR reaction incubation time was first investigated by using the optimized (with PVA+DTT) and original (without PVA+DTT) CRISPR/Cas12a biosensing system for the detection of the same amount of HP gene (12500 pM), and the fluorescence signal changes were recorded as shown in FIG. 32A. Both of the fluorescence signals increased with increasing incubation time, and the fluorescence increments between the original and optimized systems became larger with increasing incubation time. When the optimized system reaches the same level of fluorescence intensity with the original system at 120 min incubation time, less than 30 min incubation time in the optimized system was required. The inventors found that the optimized system has the capability of reducing incubation time for the detection of the same amount of HP gene target, to less than ¼ of the original system.

Further investigation of the sensitivity enhancement by the optimized system was conducted in FIG. 32B. Both of the optimized and original systems were employed to detection the HP gene with a 4 times series dilution, and the calibration curves with relevant fits are shown in FIG. 32B. The slope of optimized system was approximately 6 times of the slope of original system. The limit of detection (LOD) of optimized system and original system were calculated using the equation of LOD=3*standard derivation/slope, and the calculated LOD values of optimized system and original system were 17.7 pM and 96.3 pM, respectively. Therefore, the optimized system increased the LOD around 5.4 times. In addition, with the higher slope of optimized system, the detection resolution of optimized system also improved. An alternative investigation of detection limit was using statistical analysis (FIG. 32C). The background increase after addition of PVA+DTT was mainly due to the dispenser effect of non-ionic surfactant PVA. In the original system, only at a target concentration of 195 pM, did the P value reach 50.05. Therefore, statistic detection sensitivity for original system was 195 pM. The P value was also applied in the optimized system, and 49 pM shows a significant difference with the control (0 pM). Thus, around 4 times of detection sensitivity increased after using the optimized system.

The second application of optimized CRISPR/Cas12a biosensing system was applied in the detecting the pathogenic virus SARS-CoV-2 gene. The synthetic SARS-CoV-2 gene RNA fragment was first reverse transcribed and amplified using reverse transcription polymerase chain reaction (RT-PCR), and the final products were collected for further detection. A series of 4 times dilution of final RT-PCR products were detected using optimized and original CRISPR/Cas12a biosensing systems. The incubation time was first investigated using both systems for the detection of the same amount of target gene (200 fM) (FIG. 32D). The optimized system reaches the same level of fluorescence intensity as the original system at 120 min but in less than 30 min incubation time. Therefore, the optimized system has the capability of reducing the CRISPR/Cas incubation time to less than ¼ for reaching the same level of fluorescence intensity.

The sensitivity enhancement for optimized system was evaluated in FIG. 32E. The calculated LOD for optimized system and original system were 0.36 fM, and 1.38 fM, respectively, which increase approximately 3.8 times. Further statistical analysis of sensitivity was shown in FIG. 32F. The sensitivity (p<0.05) for original system was 3.125 fM, while the sensitivity (p<0.05) for optimized system was 0.78 fM, which is around four times increase of the detection sensitivity.

### 5. Chemical Enhanced CRISPR/Cas13a Biosensing System for the Detection of SARS-CoV-2 Gene

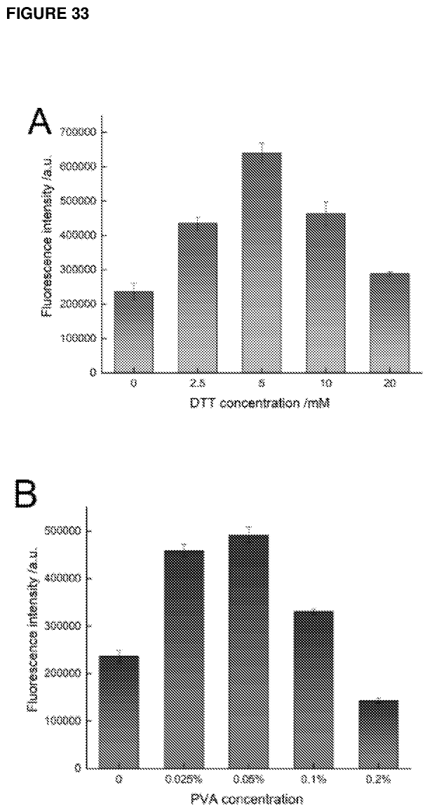

After investigation of the chemical induced trans-cleavage enhancement of CRISPR/Cas12a biosensing system, further investigation of the chemical induced trans-cleavage change of type VI CRISPR/Cas biosensing system was conducted, and Cas13a was selected as a typical representative. The optimization of chemical enhancers DTT and PVA were investigated (FIG. 33), and the optimum concentrations were determined to be 5 mM and 0.05%, respectively. The effect of combined PVA with DTT was investigated in FIG. 34A. The fluorescence signal of PVA+DTT was enhanced by a factor of approximately six times compared with control and was applied for further detection of SARS-CoV-2 gene.

In order to evaluate the reduction of incubation time, the enhanced and original CRISPR/Cas13a biosensing systems were applied for the detection of the same amount of SARS-CoV-2 gene (1000 nM) (FIG. 34B). The fluorescence intensities of both systems increased with the increased incubation time, and the optimized system took approximately 10 min to reach the same level of fluorescence intensity as the original system after a 60 m incubation. Therefore, the enhanced system is characterised by a reduced CRISPR incubation time.

Furthermore, the evaluation of sensitivity enhancement was conducted (FIGS. 34 C&D). Both systems were applied for the detection of different concentrations of SARS-CoV-2 gene. The calibration curves with lines of best fit are shown in FIG. 34C. The LOD of original and optimized systems were 61.3 pM and 11.9 pM, respectively. Therefore, the LOD of optimized system increased around 5 times with the application of chemical enhancers. Further evaluation of the sensitivity enhancement was conducted in FIG. 34D. The statistically determined detection limit for original system was 195 pM, and 49 pM for the optimized system was 49 pM, (around 4 times increased sensitivity). Therefore, an optimized biosensing system shows a reduced incubation time with a concurrent increase of sensitivity.

### Example 6. CRISPR/Cas-Based Signal Amplification for Immunoassays

In this example, the inventors report a simple but versatile strategy to successfully integrate CRISPR/Cas-mediated biosensing into conventional immunosorbent assays as a universal and robust signal amplification module, termed CRISPR-based Universal Immunoassay Signal Enhancer (CRUISE). CRUISE does not require any additional recognition elements other than antibodies and has been established on a standard microtitre (96-well) plate.

### Materials and Methodology

Chemicals: DNase/RNase free water (ThermoFisher), phosphate buffered saline (PBS) (Sigma, 10 mM, pH=7.4), agarose (Sigma), recombinant human IFN-γ protein (R&D, 285-IF-100), recombinant Human EGFR/ErB1 protein (R&D, 1095-ER-002), recombinant human IL-13 protein (R&D, 201-LB), recombinant human IL-6 protein (R&D, 206-IL), recombinant human TNF-α protein (R&D, 210-TA), human IFN-γ quantikine ELISA kit (R&D, SIF50), plasma diluent (Abcam, ab221826), Lightning-Link Streptavidin Conjugation Kit (Abcam, ab102921), EnGen® Lba Cas12a (Cpf1) protein (New England Biolab), 10× NEB 2.1 buffer (New England Biolab), streptavidin (Sigma), bovine serum albumin (Sigma), human serum (Sigma), 10,000×SYBG Gold dye (ThermoFisher), DNA loading dye (ThermoFisher), 10 bp DNA ladder (ThermoFisher).

Antibodies were obtained from R&D Systems: human IFN-γ biotinylated antibody (BAF285), human IFN-γ monoclonal antibody (MAB285), human EGF R/ErbB1 biotinylated antibody (BAF231), Human EGF R/ErbB1 polyclonal antibody (AF231), goat IgG (H+L) PE-conjugated polyclonal donkey IgG (F0107), mouse IgG (H+L) antibody (D-201-C-ABS2), donkey anti-mouse IgG NorthernLights NL637-conjugated antibody (NL008), R&D Systems Donkey Anti-Mouse IgG (H+L) Affinity Purified PAb (D201CABS2), R&D Systems Donkey Anti-Rabbit IgG (H+L) Affinity Purified PAb (D301CABS2), R&D Systems Donkey Anti-Rabbit IgG NL557 Affinity Purified PAb (NL004).

Oligos: All designed RNA and DNA oligos were synthesized by Sango Biotech (Table 9).

TABLE 9
RNA and DNA oligos sequences

| | Oligo | | Length | Modifi- |
|---|---|---|---|---|
| Name | type | Sequence 5′-3′ | (nt) | cation |
| Guiding | ssRNA | UAA UUU CUA CUA | 42 | N/A |
| RNA | | AGU GUA GAU GGG | | |
| (gRNA) | | GGG GGU UGG UAG | | |
| | | GGU GUC | | |
| | | (SEQ ID No. 6) | | |
| Triggering | ssDNA | GAA GAC ACC CTA | 30 | 5′-biotin |
| ssDNA 1 | | CCA ACC CCC CCC | | 3′-Texas |
| | | TAA ACC | | Red; |
| | | (SEQ ID No. 17) | | |
| Triggering | ssDNA | GAA GAC ACC CTA | 30 | 5′-biotin |
| ssDNA 2 | | CCA ACC CCC CCC | | |
| | | TAA ACC | | |
| | | (SEQ ID No. 17) | | |
| Triggering | ssDNA | GAA GAC ACC CTA | 30 | N/A |
| ssDNA 3 | | CCA ACC CCC CCC | | |
| | | TAA ACC | | |
| | | (SEQ ID No. 17) | | |

### Methods

Conjugation of Antibodies with Triggering ssDNA

The streptavidin-conjugated antibodies were synthesized according to the protocol of the streptavidin conjugation kit (Abcam, ab102921). First, 1 μL of the kit modifier was gently mixed with 10 μL (1 mg mL-1) of the antibody (66.7 μmol). Then 10 μL (1 mg mL-1) streptavidin (189.4 μmol) was added into the solution and gently mixed at room temperature for 3 h. Finally, 1 μL of the quencher reagent was gently mixed into the solution for 30 min at room temperature. Afterwards, the biotinylated triggering ssDNA was applied to form the Abs-ssDNA conjugates followed by mixing of 10 μL 10 μM (100 μmol) biotinylated triggering ssDNA together with 1 μg of the previously prepared streptavidin-conjugated antibody (6.7 μmol) for 3 h at room temperature. Then the solution was centrifugation-filtered using low binding PES filter with 100 k molecular separation pores to remove the unattached triggering ssDNA. The centrifugation spin was set as 10,000 rpm for 5 mins at 4° C. Wash with PBS buffer and then resuspend the reminding residue in PBS buffer, and the collected Abs-ssDNA conjugates were stored at 4° C. for further use.

Verification of Conjugating ssDNA onto the Antibodies

In order to verify the successful conjugation of ssDNA with the antibody, first 10 μL 10 μM (100 μmol) of fluorescent biotinylated triggering ssDNA (5′ Texas Red, 3′ biotin) was mixed with 1 μg streptavidin-conjugated antibody (6.7 μmol) for 3 h at room temperature. The solution was centrifugation-filtered using a low binding PES filter with 100 k molecular separation pores at 10,000 rpm for 5 mins at 4° C. Then, the fluorescent intensity of the resuspended solution was measured. Concurrently, 10 μL 10 μM (100 μmol) of the fluorescent biotinylated triggering ssDNA was also centrifugated alone, and fluorescence of the resuspended solution was measured. The fluorescence signal was tested using an SpectraMax iD3 Multi-Mode Microplate Reader (Molecular Devices), at the excitation wavelength of 570 nm (Ex=570 nm), and at the emission wavelength of 615 nm (Em=615 nm).

### Functional Test of the Abs-ssDNA Conjugate

### a) Preparation of the CRISPR/Cas12a Reaction Mixture

For all the CRUISE detections, the CRISPR/Cas12a reaction mixture was prepared as follow: 10 μL 10 μM (100 μmol) of Cas12a protein was gently mixed with 5 μL 10 μM (50 μmol) gRNA in 3.6 mL 1×NEB 2.1 buffer. Then, 6 μL 100 μM (0.6 nmol) of pre-synthesized ssDNA linked fluorescent reporter (Table 9) was added and well mixed to form the final reaction mixture.

### b) Abs-ssDNA Triggered CRISPR/Cas12a Collateral Cleavage Activity

To verify the capability of the prepared Abs-ssDNA for triggering CRISPR/Cas12a nuclease activities, 5 μL of different concentrations of prepared Abs-ssDNA conjugate (1.67 μg mL-1, 3.33 μg mL-1, 6.67 μg mL-1, 13.33 μg mL-1) have been mixed with 100 μL of the standard CRISPR/Cas12a reaction system (10 μL 10 μM (100 μmol) Cas12a protein, 5 μL 20 μM (100 μmol) gRNA, 3.6 mL 1×NEB 2.1 buffer, 6 μL 100 μM (0.6 nmol) of fluorescent-quenched reporter). Then, a dynamic fluorescent signal intensity has been measured by iD3 plate reader at Ex=570 nm, Em=615 nm for 100 mins with 10 mins intervals.

### c) Target Specific Binding Affinity of Abs-ssDNA Conjugate

To verify the capability of the prepared Abs-ssDNA for binding to its target analyte, a competitive binding test has been performed. After the analyte has been fixed onto the 96-well plate surface, 100 μL of 4 μg mL-1 Abs-ssDNA or 100 μL of 4 ug mL-1 Abs-ssDNA/pristine Abs mixture (Abs-ssDNA/pristine=1:1) has been added to the 96-well plate with analyte for 1 hour at room temperature. After 3 times of 350 μL PBS washing, 100 μL of standard CRISPR/Cas12a reaction system has been added, and incubated at room temperature for 1 hour. The fluorescent signal then been tested by iD3 plate reader at Ex=570 nm, Em=615 nm.

To verify the capability of the prepared Abs-ssDNA for binding to its target analyte, a competitive binding test has been performed. After the analyte has been fixed onto the 96-well plate surface, 100 μL of 4 μg mL-1 Abs-ssDNA or 100 μL of 4 ug mL-1 Abs-ssDNA/pristine Abs mixture (Abs-ssDNA/pristine=1:1) has been added to the 96-well plate with analyte for 1 hour at room temperature. After 3 times of 350 μL PBS washing, 100 μL of standard CRISPR/Cas12a reaction system has been added, and incubated at room temperature for 1 hour. The fluorescent signal then been tested by iD3 plate reader at Ex=570 nm, Em=615 nm.

### d) Universality of Abs-ssDNA Conjugate Preparation

To verify the universality of making the Abs-ssDNA conjugate, additional 3 different antibodies (anti-IL-1p, anti-IL-6, anti-EGFR) has been used to form the Abs-ssDNA separately followed the same previous described protocol. Then, 5 μL of each prepared Abs-ssDNA has been mixed with 100 μL of standard CRISPR/Cas12a reaction mixture. After 1-hour incubation at room temperature, the fluorescent signal has been tested by iD3 plate reader at Ex=570 nm, Em=615 nm.

### CRUISE as Primary Antibody for Analytes Detection

a) Coating Capture Antibody onto 96-Well Plate

Firstly, A high-binding polystyrene flat bottom 96-well plate was coated with 100 μL10 μg mL-1 streptavidin at 4° C. for 2 h. After 3 times of 1×PBS wash, followed by the application of 0.5 mg mL-1 BSA blocking solution at room temperature for 1 h. Afterwards, 4 μg mL-1 of biotinylated capture antibody was immobilized on this polystyrene plate at room temperature for 1 h. After 3 times of 1×PBS wash, the sensing interface thus prepared was stored at 4° C. for further use.

### b) Standard Sandwich CRUISE Protocol

For each of the CRUISE reaction, after the sensing interface was fabricated on the 96-well plate, 100 μL of sample solution (where the sample could be PBS, serum or plasma) was added at room temperature for 1 h, followed by washing 3 times with 1×PBS. Then, 100 μL 4 μg mL-1 of the detection Abs-ssDNA conjugate was applied at room temperature for another 1 h. After washing 3 times in 1×PBS, 100 μL of the prepared standard CRISPR/Cas12a reaction mixture was added for signal generation and incubated at room temperature for 30 mins. The fluorescence signal was detected by the iD3 plate reader with Ex=570 nm and Em=615 nm.

c) CRUISE Protocol without Capture Antibody

100 μL of different concentrations of analyte protein (IFN-γ: 0.2, 2, 20, 200 and 2000 pg mL-1) has firstly been applied directly onto a high-binding polystyrene flat bottom 96-well plate, and followed with 1.5 hour incubation at room temperature. Then, 3 times of 1×PBS wash has applied. Afterwards, 200 μL 0.5 mg mL-1 BSA blocking solution has applied and incubated at room temperature for 1 h. After 3 times of 1×PBS wash, 100 μL 4 μg mL-1 of the detection Abs-ssDNA conjugate was applied at room temperature for another 1 h. After washing 3 times in 1×PBS, 100 μL of the prepared standard CRISPR/Cas12a reaction mixture was added for signal generation and incubated at room temperature for 30 mins. The fluorescence signal was detected by the iD3 plate reader with Ex=570 nm and Em=615 nm.

### d) Application of CRUISE for Cytokines Detection

By applying cytokines (IFN-γ, EGFR) as examples, the above standard protocol of CRUISE protocol was applied in the pre-made 96-well plate with target cytokine and its corresponding Abs-ssDNA conjugate. Testing samples were prepared by spiking human IFN-γ analyte into either 1×PBS or serum at different concentrations between 1 ng mL-1 and 1 fg mL-1 in a 10× serial dilution. Then, 100 μL of each prepared sample was applied, followed by the standard protocol of CRUISE for detection of target analytes.

iCRUISE as Secondary Antibody for Analyte Detection

To demonstrate the feasibility of using CRUISE as the secondary antibody in immunoassay, the 96-well plate sensing interface for IFN-γ (EGFR) and its corresponding Abs-ssDNA conjugate were firstly prepared following the same method as above. Then, 100 μL of the prepared IFN-γ (EGFR) samples with concentrations between 1 ng mL-1 and 1 fg mL-1 were added at room temperature and incubated for 1 h. After 3 times washing with 1×PBS, 100 μL 4 μg mL-1 of pristine IFN-γ detection antibody was applied at room temperature for 1 h, followed by 3 cycles of 1×PBS washing. Afterwards, 100 μL of 10 μg mL-1 anti-IgG Abs-ssDNA conjugate was applied at room temperature and incubated for another 1 h. After 3 times washing with 1×PBS, 100 μL of pre-made CRISPR/Cas12a reaction mixture was added at room temperature and incubated for 30 mins for signal generation. The fluorescence signal was then detected using the iD3 plate reader at Ex=570 nm and Em=615 nm.

### Calculation of the Limits of Detection (LOD)

Each of the generated calibration curve was fitted with 4 parametric logistic plots and its LOD was calculated by the application of the standard equation “LOD=3×SD/k”, where the SD stands for the standard deviation of the background signal, and the k stands for the slope of the linear curve.

### Results

### 1. Principle of the CRUISE Biosensing Platform

The CRUISE platform is established on the basis of a common immunoassay format, where antibody-analyte interaction supports the highly specific and stable binding. The schematic of the CRUISE platform is shown in FIG. 35. The antibody, responsible for linking the CRISPR/Cas12a enzymatic activity with signal reporting, is conjugated with a short single strand DNA (ssDNA) oligonucleotide through the streptavidin-biotin binding to form the antibody-ssDNA (Abs-ssDNA) conjugate. This attached short ssDNA is specifically designed with a complementary sequence to the guiding RNA of the subsequently applied CRISPR/Cas12a RNP, and is therefore capable of activating the Cas12a collateral cleavage activity. Then, this indiscriminate DNA nuclease activity can be harnessed to continuously and rapidly unquench the surrounding fluorescent-quenched ssDNA reporters to generate a highly amplified fluorescent signal for the final detection.

After a proper Abs-ssDNA conjugate has been prepared, it can be integrated into conventional immunoassay formats through two different approaches: by being applied as the primary antibody in direct immunoassays, or alternatively as the secondary antibody in indirect immunoassays. In the first approach, the Abs-ssDNA has direct contact with the targeting analyte. It can be either applied directly for target recognition as the stain-alone antibody in a biosensing system or applied as the detection antibody to form a typical antibody sandwich structure. In the alternative approach, the Abs-ssDNA conjugate has been used as a secondary antibody to reveal the presence of another antibody, by recognizing the Fc fragment of a detection antibody. In either way, after a proper separation procedure to remove the unbound Abs-ssDNA conjugates, the subsequently added CRISPR/Cas12a RNPs with fluorescent quenched reporters can be activated by the triggering ssDNA on the Abs-ssDNA, to produce the amplified fluorescent signal. The magnitude of the fluorescent signal correlates with the amount of targeted analyte.

### 2. Forming the Abs-ssDNA Conjugate

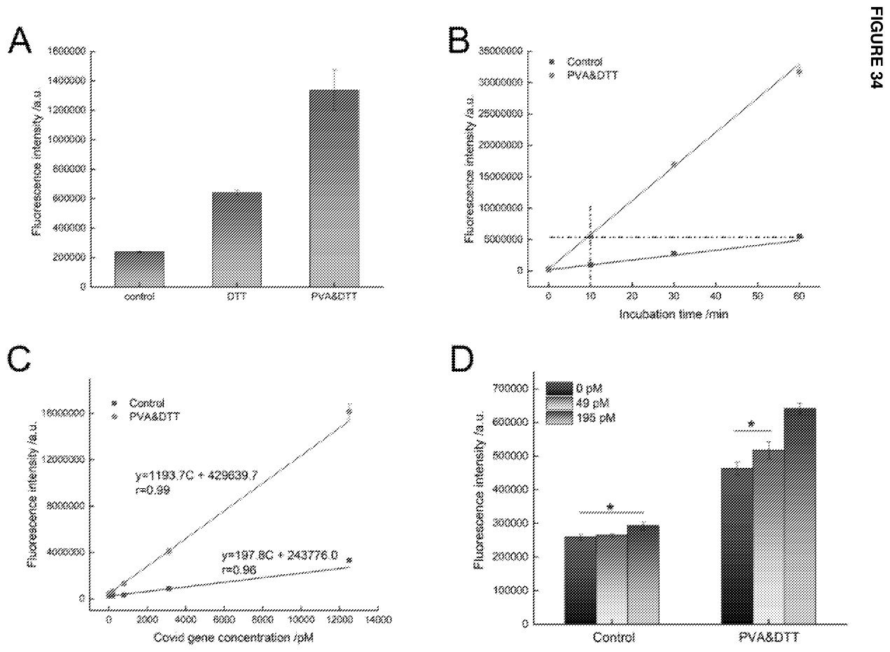

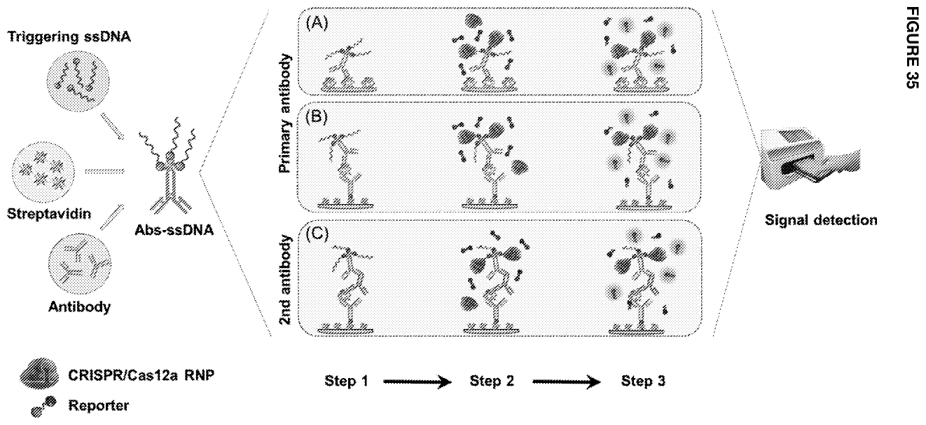

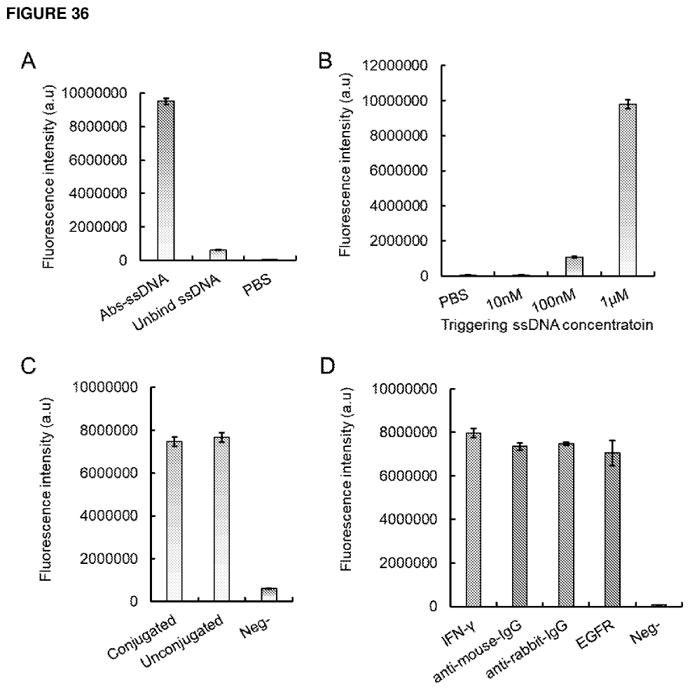

To make the CRUISE platform more accessible and ready-to-use for wide range of users, a commercial conjugation kit has been applied to link the streptavidin onto the primary amine groups of the antibody, and then forming the final Ab-ssDNA conjugate. This was achieved by adding of a 30 nt synthesized ssDNA oligonucleotide, with 5′-Texas Red fluorophore and 3′-biotin labelling. After centrifugation-based filtration, the unbound ssDNAs were removed due to their much lower molecular weight compared to the antibody. Comparing with the removed unbound ssDNA, the significantly increased fluorescent signal on the remaining antibodies indicated the successful formation of the Abs-ssDNA conjugates (FIG. 36A). The increase of the fluorescent signal intensity with increasing concentration of triggering ssDNA further proved the binding activity between antibody and the triggering ssDNA (FIG. 36B).

### 3. Abs-ssDNA Conjugate Derivate CRISPR/Cas12a Activation

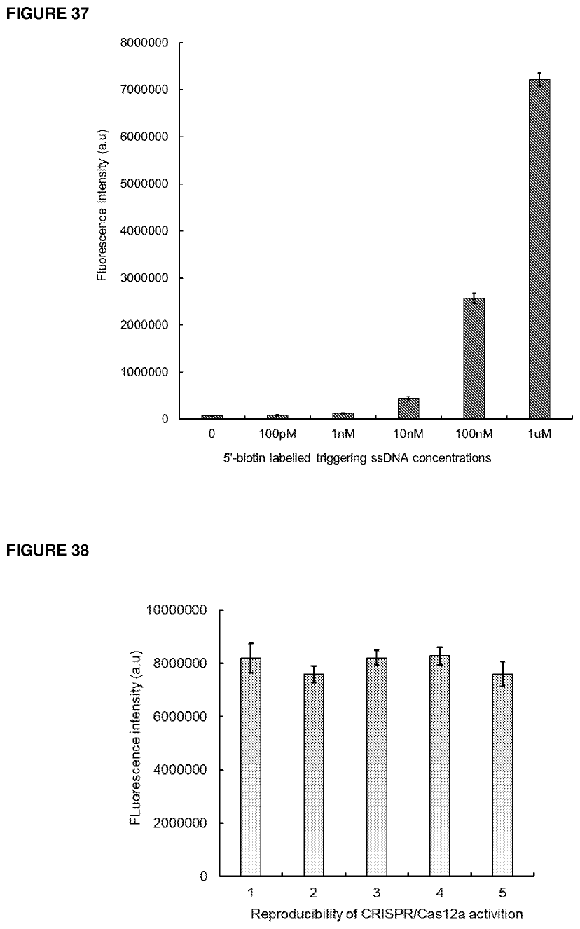

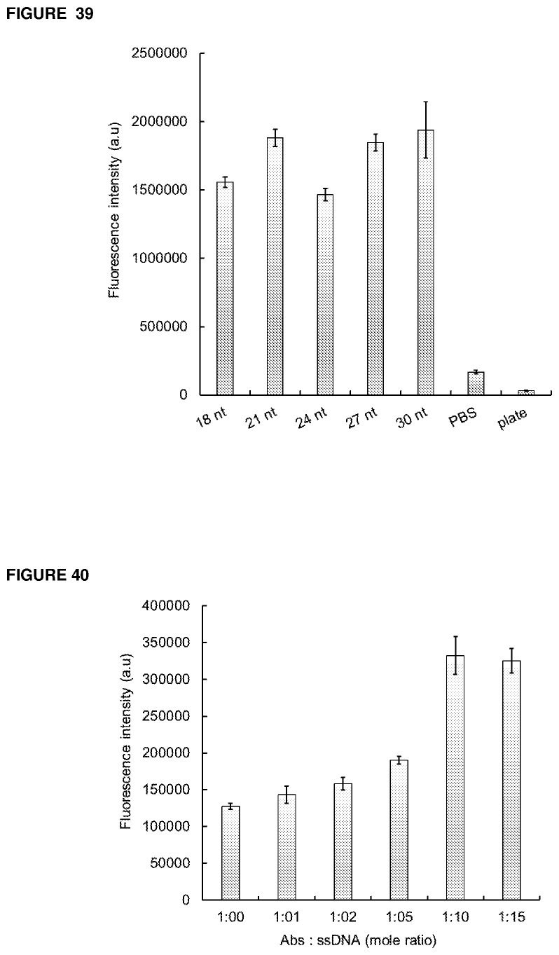

The Cas12a activation capability of designed triggering ssDNA with 5′ biotin labelling has been tested firstly, and the results indicated an expected positive correlation between fluorescent signal intensity and the concentration of triggering ssDNA (FIG. 37). After the triggering ssDNA has been conjugated onto the antibody, a competitive binding assay has been conducted between the conjugated Abs-ssDNA and a pristine antibody in order to verify the antibody affinity after conjugation. The results show no significant difference in target analyte binding, which indicates that specific target recognition capability in our Abs-ssDNA conjugate remained intact (FIG. 36C). To demonstrate that the antibody and triggering ssDNA conjugation is a widely applicable strategy, four different antibodies (anti-IFN-γ, anti-mouse-IgG, anti-rabbit-IgG and anti-EGFR) have been used to prepare the Abs-ssDNA conjugates. All have been found capable to effectively activate the downstream CRISPR/Cas12a and yield a ≥330 times increase of fluorescent signal intensity, comparing to negative control, after 1-hour room temperature incubation (FIG. 36D). In addition, the reproducibility of Abs-ssDNA induced CRISPR/Cas12a activation has also been tested, and the results show no significant difference in activation efficiency as seen in FIG. 38. CRISPR/Cas12a activation under different lengths of triggering ssDNA, including 18, 21, 24, 27 and 30 nt, has also been investigated. The results indicated no significant correlation between CRISPR/Cas12a activity intensity and the length of triggering ssDNA within the tested range (FIG. 39). Although the length of triggering ssDNA is not critical in the reaction, a minimum of 18 nt lengths is reported to be necessary to maintain the functionality of Cas12a nuclease.

### 4. CRUISE Platform Optimization

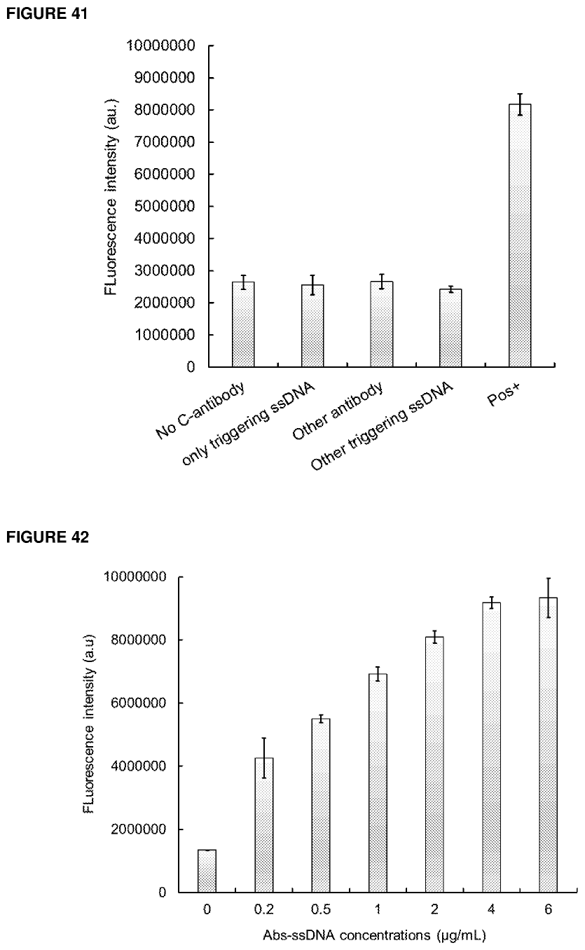

In order to establish the protocol for the preparation of the Abs-ssDNA conjugate, the concentration of antibody and triggering ssDNA has been investigated first, and the results show saturation of fluorescence intensity at the molar ratio of antibody/triggering ssDNA reaching 1:10 (FIG. 40). This closely corresponds to the calculated value of 11.36 based on manufacturer instruction in the conjugation kit (one antibody with 2.84 streptavidin attached). This optimal ratio was applied for all subsequent tests. After the Abs-ssDNA conjugates were prepared, the CRUISE idea has firstly been demonstrated through an antibody-based sandwich immunoassay, where a significant fluorescent signal increase can be observed only in the presence of all the designed components, including the capture antibody, target analyte, and the Abs-ssDNA conjugate (FIG. 41). Then, the application of CRUISE in an immunoassay has been optimized with respect to various factors.

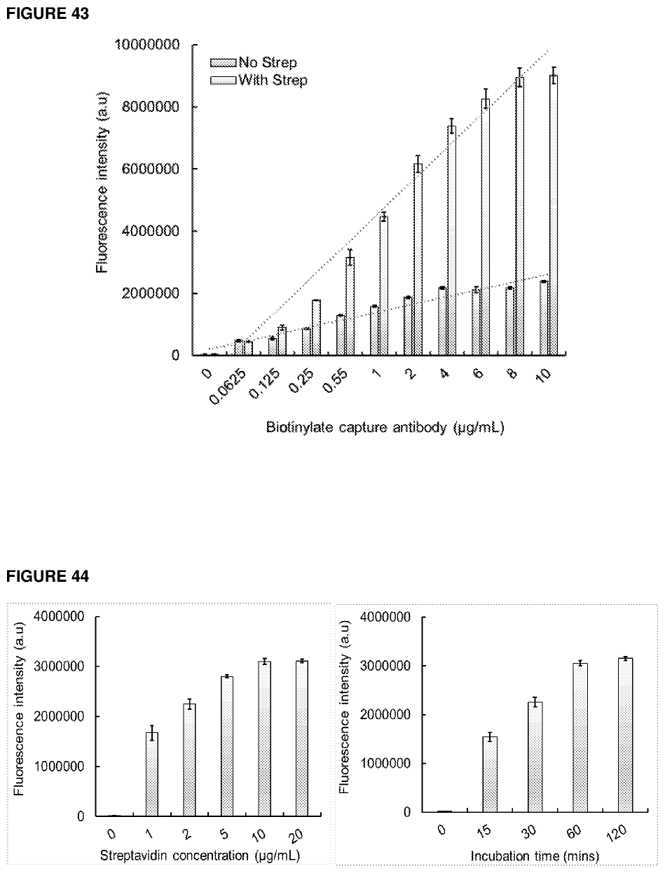

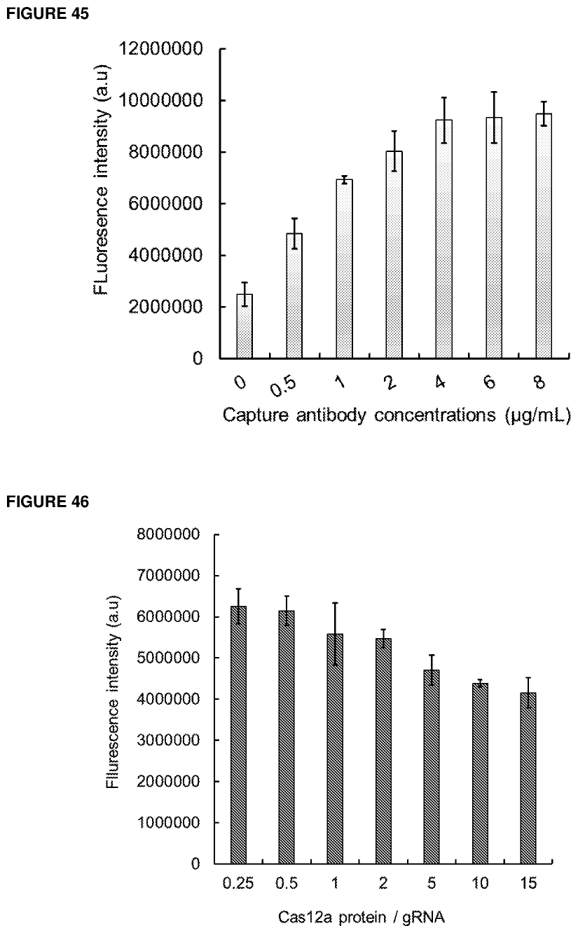

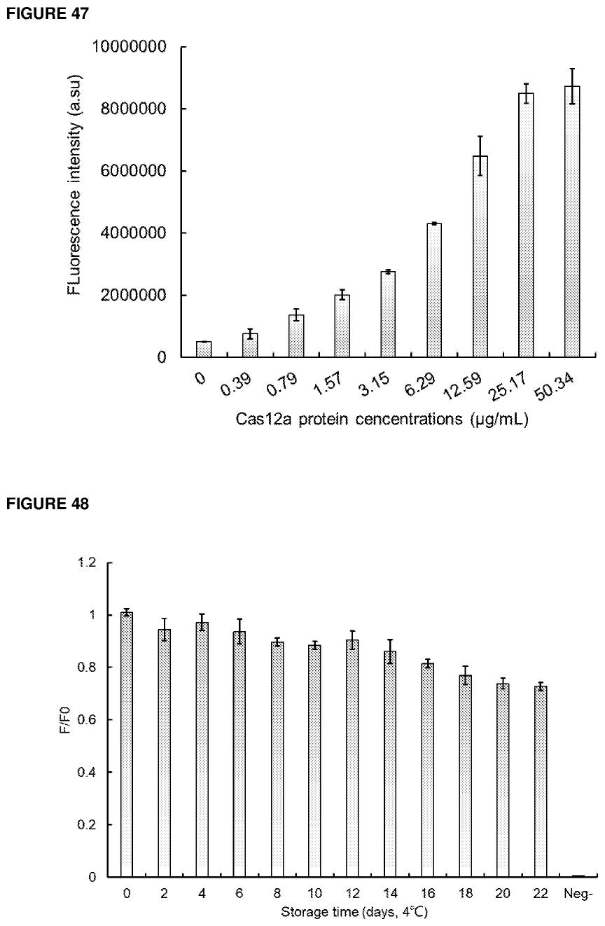

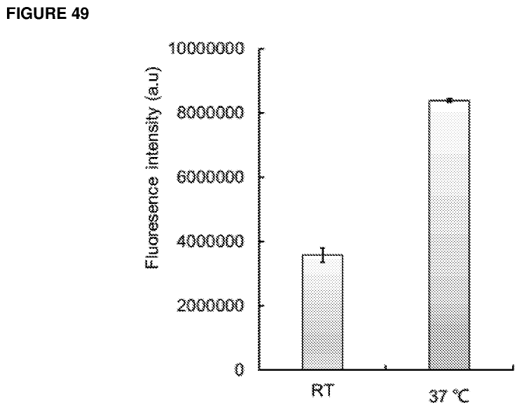

Firstly, the optimal Abs-ssDNA concentration was investigated using a sandwich immunoassay (FIG. 42). The fluorescence signal reached the peak as the Abs-ssDNA concentration increased to the value of 4 μg mL-1, which was selected as the optimum concentration of Abs-ssDNA. In the presence of capture antibody, the capture antibody coating efficiency, on the high-binding 96 well plate, without the help of streptavidin is reduced ˜75% when comparing with streptavidin coated, which highlights the necessity of applying streptavidin (FIG. 43). The loading capacity of streptavidin reaches maximum value at concentration of 10 μg mL⁻¹ with 1-hour incubation at room temperature (FIG. 44). Subsequently, with the increase of the capture antibody concentrations, the fluorescence signal increased and reached a plateau at 4 μg mL⁻¹ (FIG. 45). Thus, 4 μg mL⁻¹ was chosen as the optimum concentration for applying capture antibody to the plate. The effects of the CRISPR/Cas12a/gRNA ratio to activation efficiency have also been investigated. The data show a decrease in the activation efficiency when increasing the ratio, and indicate that the ratio lower then 2:1 is preferred (FIG. 46). Next, the Cas protein concentration was also investigated for its activity variation (FIG. 47). The increase of Cas protein concentration from 0 to 50 μg mL⁻¹ has led to the increase of fluorescence signal intensities before reaching a plateau at 25 μg mL⁻¹. As a result of costs/effects consideration, we used the Cas concentration of 4.2 μg mL⁻¹ for all subsequent tests, which is much lower than the tested optimal concentration, but already yields maximum practical sensitivity up to 1 fg mL⁻¹ in our subsequent tests. Accordingly, the stability of the implemented CRISPR/Cas12a fluorescence amplification methodology has also been investigated. The pre-made CRISPR/Cas12a reaction mixture (including gRNA loaded Cas12a RNP and fluorescent quenched reporters) and triggering ssDNA has been stored at 4° C., and the activation efficiency was tested as a function of storage time. The results show a slightly decreased trend within the tested time range, but the activation efficiency remained high, at >90% after the first week and >70% after 3 weeks (FIG. 48). Although a significantly higher increase in fluorescent signal output has been observed when reaction temperature was raised to 37° C. (FIG. 49), room temperature was still selected in this study so that additional heating is not required.

### 5. Primary Antibody-Derived CRUISE

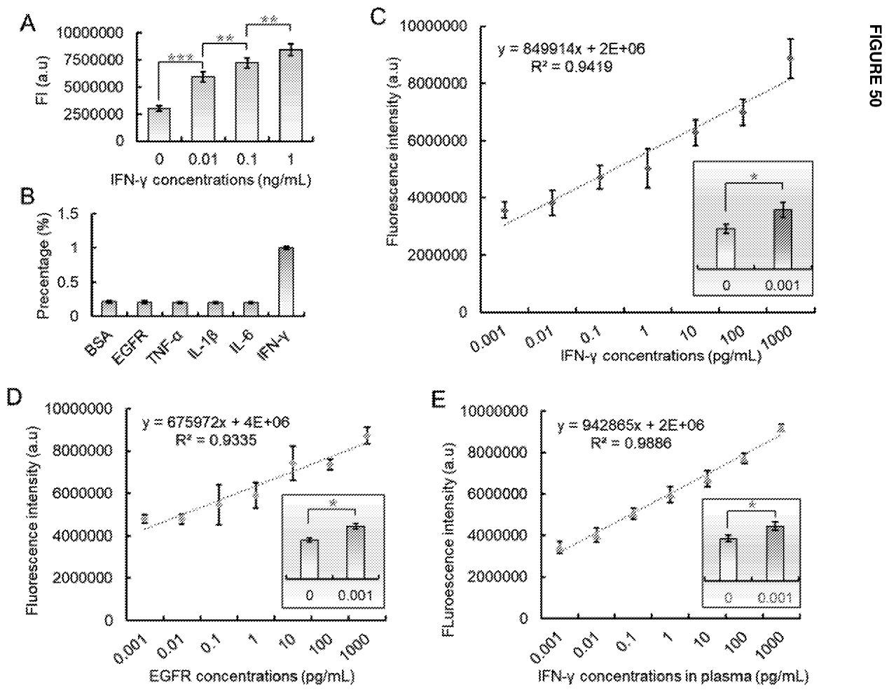

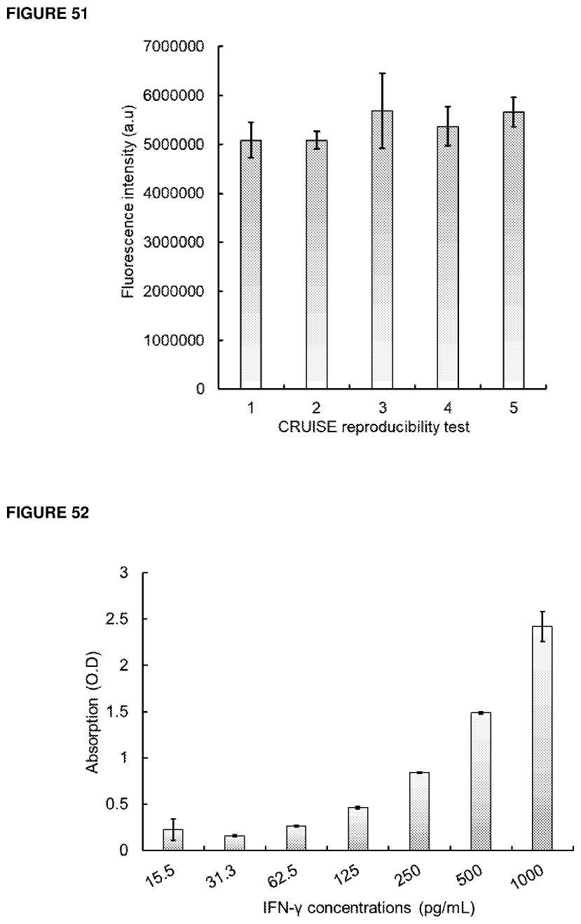

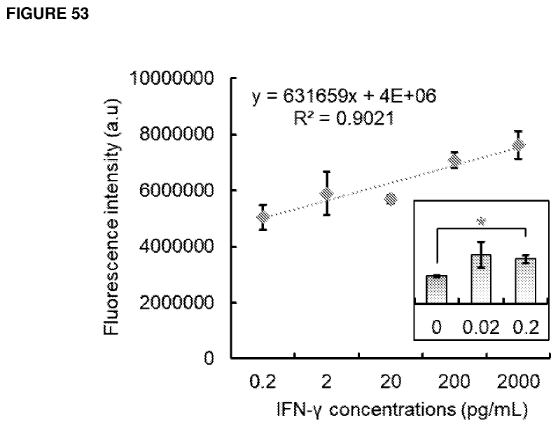

After the IFN-γ Abs-ssDNA conjugate has been prepared, and applied as the primary detection antibody in a typical sandwich immunoassay, a positive correlation between different IFN-γ concentrations (0.01, 0.1 and 1 ng mL⁻¹) and the CRUISE fluorescent signal output has been demonstrated (FIG. 50A), which indicated the potential for quantitative analyte detection. The system specificity test has also been conducted by using 5 different interfering small proteins, including 4 additional cytokines (IL-1p, IL-6, TNF-α, EGFR) and BSA. Comparing to the presence of other interfering analytes, the significantly higher fluorescent signal intensity in the positive IFN-γ sample demonstrated the highly specific response of our CRUISE system to its target analyte only (FIG. 50B). In addition, the system reproducibility has also been evaluated by detection of IFN-γ in 5 independent tests under identical conditions. The results indicate satisfactory repeatability as no significant intensity differences have been observed in the output signal (FIG. 51). For the detection of IFN-γ, our CRUISE platform can reach a 1 fg mL⁻¹ sensitivity with 6 logs of linear range from 1 fg mL⁻¹ to 1 ng mL⁻¹, and the calculated Limit of Detection (LOD) is 5.65 fg mL⁻¹ (FIG. 50C). In comparing to a commercial IFN-γ ELISA kit, which capable of detecting the analyte concentrations higher than 31.3 pg mL⁻¹ (FIG. 52), our CRUISE is three orders of magnitude more sensitive. Furthermore, to demonstrate the universality of our CRUISE platform for detecting other target analytes, another small protein cytokine, EGFR, has been used to test the same CRUISE protocol with its own anti-EGFR Abs-ssDNA conjugate. The results shown the same 1 fg mL⁻¹ sensitivity with 6 logs linear range from 1 fg mL⁻¹ to 1 ng mL⁻¹, and calculated LOD of 5.05 fg mL⁻¹ (FIG. 50D), which indicates the potential of our system to detect other targets by simply adjusting the antibody type. Both the detection performance of these analytes was similar and significantly improved compared to the claimed performance of a typical commercial ELISA kit. Additionally, the capability of CRUISE for non-sandwich immunoassay has also been demonstrated, a similar test has been performed to detect IFN-γ, but without the presence of capture antibody, which yielded a 200 fg mL⁻¹ practical sensitivity along with 4 logs of linear range (FIG. 53). Although this is slightly lower than the sandwich-based CRUISE tests, it is still higher than the majority of commercial IFN-γ immunoassays. To further demonstrate the potential of CRUISE to work on clinically relevant complex samples, the plasma diluent (product #ab221826 from Abcam) was directly spiked with different concentrations of IFN-γ to simulate a common clinical diagnostic environment, and then tested by CRUISE. The result show uncompromised performance for analyte detection in this plasma related sample, and exhibits the same 1 fg mL⁻¹ sensitivity with 6 logs linear range from 1 fg mL⁻¹ to 1 ng mL⁻¹, and the calculated LOD is 4.56 fg mL⁻¹ (FIG. 50E).

6. Secondary Antibody Derived CRUISE (iCRUISE)

In the primary antibody-based approach, the success of CRUISE relies on the formation of a functional Ab-ssDNA conjugated with a suitable detection antibody. The standard triggering ssDNA and antibody conjugation method of CRUISE utilise the primary amine group of the antibody, which may represent a limitation when the amine groups are abundant within the antigen binding region. Consequently, the triggering ssDNA binding may cause a significant loss of antigen-binding capability of the antibody³⁵. Therefore, working on the secondary antibody for fabricating a functional anti-IgG Abs-ssDNA conjugate may provide an alternative way to bypass such limitation, while retaining the versatility for application to different immunoassays with the targeted IgG with the same antibody source.

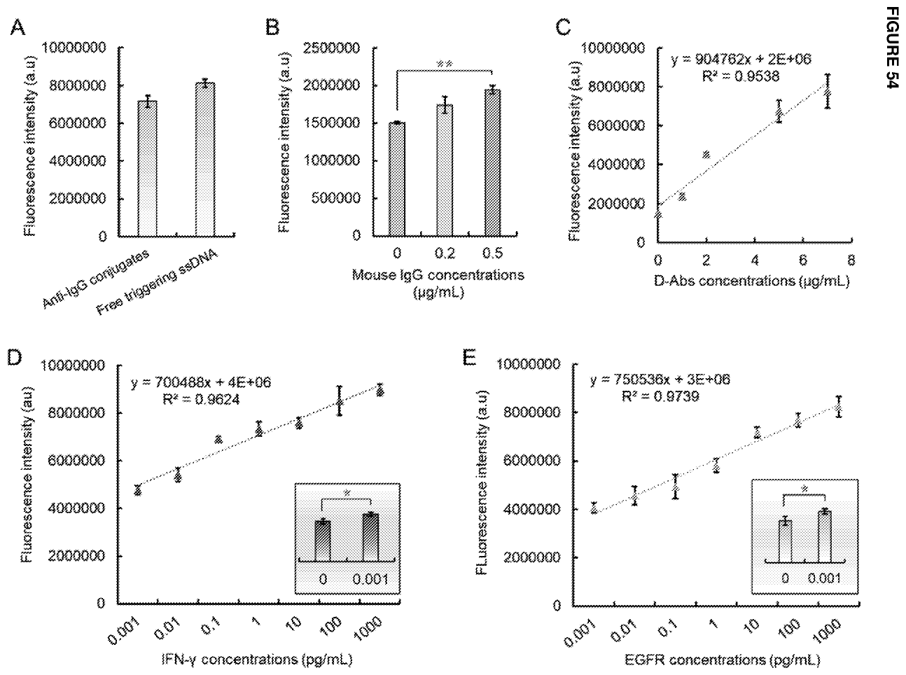

To demonstrate the feasibility of iCRUISE in immunoassay, an anti-IgG Abs-ssDNA conjugate has been firstly prepared by the same protocol as described previously. The CRISPR/Cas12a activation test shown that thus prepared anti-IgG Abs-ssDNA conjugate can efficiently trigger the CRISPR/Cas12a RNPs nuclease activity in comparison to the free triggering ssDNA alone (FIG. 54A). Also, with the increase of the anti-IgG concentrations (0.2 μg mL⁻¹, 0.5 μg mL⁻¹) in recognizing the targeted antibody, the fluorescent signal intensity has also increased significantly (FIG. 54B), which verifies that target binding affinity for the prepared anti-IgG Abs-ssDNA was retained. As the detection antibody here has become the target of iCRUISE, a linear correlation between the target detection antibody and the anti-IgG Abs-ssDNA was observed within the range of 1 μg mL⁻¹ to 7 μg mL⁻¹ (FIG. 54C), which indicates the potential of realise quantitative detection. Taking IFN-γ as the example again, the antibody-analyte sandwich structure was firstly formed by the detection antibody without ssDNA conjugation, then our iCRUISE has been applied. We found that practical sensitivity was uncompromised, down to 1 fg mL⁻¹ with the same 6 logs linear range from 1 fg mL⁻¹ to 1 ng mL⁻¹, and 6.95 fg mL⁻¹ of calculated LOD (FIG. 54D). To demonstrate that this iCRUISE approach is also reproducible in additional antibody-analyte sandwich structures, the same iCRUISE protocol has been used for targeting the detection antibody of the antibody-EGFR sandwich, which shows similar system performance (FIG. 54E). As the iCRUISE uses an anti-IgG Abs-ssDNA conjugate, it has the potential to be directly transferred to other similar immunoassays for targeting antibodies with the same IgG Fc source, without additional modification or optimization. This makes the iCRUISE approach to constitute a single, ready-to-use component that is directly applicable to a wide range of immunoassays.

### Discussion

Without significantly changing a typical sandwich immunoassay protocol, it can be applied to a primary antibody to reach practical sensitivity down to 1 fg mL⁻¹ (˜50 aM), along with a wide linear range of six orders of magnitude, for detecting disease-related small proteins, such as cytokines including IFN-γ and EGFR. This is 5-6 orders increase in sensitivity compared to other reported CRISPR/Cas12a-based non-nucleic acid detection systems without antibody. When comparing the IFN-γ detection results to a commercial IFN-γ ELISA kit, CRUISE exhibited 3 orders of increase in both sensitivity and linear range, while retaining similar specificity, simplicity and cost. Alternatively, CRUISE has also been applied as a secondary antibody in indirect immunoassays, termed indirect-CRUISE (iCRUISE). The results of iCRUISE yielded the same 1 fg mL⁻¹ practical sensitivity and 6 orders of magnitude linear range for IFN-γ and EGFR detection. By directly bridging between CRISPR/Cas12a and immunoassays without additional recognition molecules and complex internal synthesis schemes, CRUISE benefits from well-established availability of reliable and reproducible commercial antibodies, and it provides a simple, reliable, and versatile way for extending the CRISPR/Cas-based biosensing to a wide range of non-nucleic acid analytes. It is also an affordable and user-friendly solution for upgrading the existing immunoassay systems to meet the diagnostic needs for highly sensitive detection.

### Example 7. CRISPR/Cas12a-Based ELISA Sensitivity Amplifier (CES-Amplifier)

This Example is directed to use of the CRISPR biosensing approach to enhance the sensitivity of commercial ELISA kits which use HRP but have otherwise unknown chemistry. While exemplified in commercial ELISA kits utilising HRP, the skilled person will readily appreciate that the CES-amplifier approach may be used with antibodies directed to other labels/enzymes commonly employed utilised in ELISA assays. The central component is a specifically designed conjugate of a short single strand DNA (ssDNA) and anti-HRP antibody. This conjugate is able to activate CRISPR/Cas12a without compromising its activation efficiency for the conjugated ssDNA, while the affinity for the conjugated antibody is also largely unaffected.

### Materials and Methodology

### Materials and Reagents

Phosphate buffered saline (PBS) (Sigma, 10 mM, pH=7.4), Lightning-Link Streptavidin Conjugation Kit (Abcam, ab102921), streptavidin (Sigma), recombinant human IFN-γ protein (R&D, 285-IF-100), Donkey Anti-Mouse IgG (H+L) Affinity Purified PAb (B&D, D201 CABS2), Donkey Anti-Rabbit IgG (H+L) Affinity Purified PAb (B&D, D301 CABS2), Human IFN gamma ELISA Kit (Abcam, ab174443), EnGen® Lba Cas12a (Cpf1) protein (New England Biolab), 10×NEB 2.1 buffer (New England Biolab), bovine serum albumin (Sigma), GelRed DNA dye (ThermoFisher), 6×DNA gel loading dye (ThermoFisher), 10 bp DNA ladder (ThermoFisher), agarose (Sigma), synthesized RNA and DNA oligos (Sango Biotech Ltd., Table 10).

TABLE 10
RNA and DNA oligos sequences
(Synthesized and modified by Sangon. Ltd.)

| | Oligo | | Length | Modifi- |
|---|---|---|---|---|
| Name | type | Sequence 5′-3′ | (nt) | cation |
| Guiding | ssRNA | UAA UUU CUA CUA | 42 | N/A |
| RNA (gRNA) | | AGU GUA GAU GGG | | |
| | | GGG GGU UGG UAG | | |
| | | GGU GUC | | |
| | | (SEQ ID No. 6) | | |
| Triggering | ssDNA | GAA GAC ACC CTA | 30 | 5′-biotin |
| ssDNA 1 | | CCA ACC CCC CCC | | 3′-Texas |
| | | TAA ACC | | Red |
| | | (SEQ ID No. 17) | | |
| Triggering | ssDNA | GAA GAC ACC CTA | 30 | 5′-biotin |
| ssDNA 2 | | CCA ACC CCC CCC | | |
| | | TAA ACC | | |
| | | (SEQ ID No. 17) | | |
| Triggering | ssDNA | GAA GAC ACC CTA | 30 | 3′-Texas |
| ssDNA 3 | | CCA ACC CCC CCC | | Red |
| | | TAA ACC | | |
| | | (SEQ ID No. 17) | | |
| Triggering | ssDNA | GAA GAC ACC CTA | 30 | N/A |
| ssDNA 3 | | CCA ACC CCC CCC | | |
| | | TAA ACC | | |
| | | (SEQ ID No. 17) | | |
| ssDNA | ssDNA | TTATT | 5 | 5′-Texas |
| reporter | | | | Red; |
| | | | | 3′-BHQ2 |

### Methods

### 1. Verification of the CRISPR/Cas12a Collateral Cleavage

For preparing the CRISPR/Cas12a reaction mixture: 3.6 mL 1×NEB 2.1 buffer was diluted by using milliQ water, and followed by gently mixed with 5 μL 10 μM (50 μmol) gRNA, 1 μL 100 μM (100 μmol) of Cas12a protein, and 6 μL 100 μM (0.6 nmol) of pre-synthesized probe (Table 10) thoughtfully. The prepared CRISPR/Cas12a reaction mixture was stored at 4° C. before use.

For verifying the CRISPR/Cas12a collateral cleavage, 5 μL of each triggering component (either free triggering ssDNA or ssDNA-Abs conjugates) was mixed with 95 μL of prepared CRISPR/Cas12a reaction mixture. The reaction was set at room temperature for an endpoint measurement at 60 mins or a dynamic fluorescence signal intensity measurement by iD3 plate reader (Molecular Devices, LLC.) at Excitation wavelength (Ex)=570 nm, Emission wavelength (Em)=615 nm.

2. Conjugation of ssDNA and Antibody

The antibodies (goat anti-HRP antibody, rabbit anti-HRP antibody) were firstly conjugated with streptavidin according to the instruction of the streptavidin conjugation kit (Abcam, ab102921). Briefly, 1 μL of the modifier from the conjugation kit was gently mixed with 10 μL of the antibody (1 mg/mL, 66.7 μmol). Followed by adding of 10 μL streptavidin (1 mg/mL, 189.4 μmol) and gently mixed at room temperature. After 3 h, 1 μL of the quencher solution was gently mixed into the solution, and set at room temperature for 30 min. Afterwards, 10 μL 10 μM (100 μmol) company-synthesized biotinylated triggering ssDNA was mixed with 1 μg (6.7 μmol) streptavidin conjugated antibody at room temperature for 3 h for the ssDNA-Abs conjugate formation.

Then, the unattached free triggering ssDNA was removed by centrifugation-based filtration with a low binding PES filter (100 kD) at 10,000 rpm for 5 mins at 4° C. Then, wash step was applied by 1×PBS buffer with repeated centrifugation, and the formed ssDNA-Abs conjugate was resuspended in 1×PBS buffer with 10 mg/mL BSA. When the biotinylated triggering ssDNA was labelled with Texas Red (Table 10), the fluorescence intensity changes in the resuspended solution represents the successful conjugation of ssDNA-Abs conjugate. The collected ssDNA-Abs conjugates were stored at 4° C. for further use.

3. Electrophoretic Mobility Shift Assay for Verifying the ssDNA-Abs Conjugate Formation

We used the electrophoretic mobility shift assay (EMSA) to verify the formation of the ssDNA-Abs conjugate. To this aim, 10 μL of the prepared ssDNA-Abs conjugate was mixed with 2 μL 6×DNA gel loading dye, and then loaded onto 2% agarose gel with premixed SYBR Gold DNA dye (1 μL 10,000× into 30 mL agarose gel). 5 μL 10 bp DNA ladder was applied on the gel as the molecule weight reference, and set as constant voltage electrophoresis at 120 V for 90 mins. Afterwards, the gel image was acquired by Gel Doc+XR image system (Bio-Rad).

4. Target Binding Affinity of the Anti-HRP ssDNA-Abs Conjugate to HRP Labelled Antibody
a) 100 μL 4 μg/mL anti-HRP ssDNA-Abs conjugate has been added onto the high-binding 96-well plate, and incubated at room temperature for 1 h. After 3 times 1×PBS wash, 100 μL HRP labelled antibody from Abcam ELISA kit was applied with the recommended concentration of the ELISA kit instruction and incubated at room temperature for 1 h. Afterwards, the excess antibodies were removed by 3 times 1×PBS wash, and followed by applying 100 μL TMB substrate as instructed by the ELISA kit instruction for colorimetric signal generation. After 15 mins incubation at room temperature under dark, the absorbance has been measured by iD3 plate reader (Molecular Devices, LLC.) at 440 nM.
b) 100 μL HRP labelled antibody from the commercial ELISA kit was added on the high-binding 96-well plate with the recommended concentration of the ELISA kit instruction and incubated at room temperature for 1 h. 100 μL antibody diluent without HRP-antibody has been used as the control (0 μg/mL) and an empty plate (EP) has also been used as a background control. After 3 times 1×PBS wash, 100 μL prepared anti-HRP ssDNA-Abs conjugate was applied at room temperature for 1 h. Afterwards, 3 times 1×PBS wash was applied and followed by applying 100 μL CRISPR/Cas12a reaction mixture and incubated at room temperature for 1 h. The fluorescence signal was then measured by iD3 plate reader (Molecular Devices, LLC.) with Ex=570 nm and Em=615 nm.

### 5. The Effects of Antibody Dilution Buffer for CRISPR/Cas12a Activity

The CRISPR/Cas12a reaction mixture was prepared as described in Method 1. Then, 100 uL prepared CRISPR/Cas12a reaction mixture was activated by adding 5 μL 1 μM triggering ssDNA. The fluorescence signal was measured continuously by iD3 plate reader (Molecular Devices, LLC.) with Ex=570 nm and Em=615 nm. Afterwards, 5 μL antibody dilution buffer from the Abcam IFN-γ ELISA kit was directly mixed into the activated CRISPR/Cas12a reaction mixture at 15 mins reaction time, and the fluorescence signal was keep measuring continuously. 5 μL 1×PBS solution was also added as the control to evaluate the signal changes.

### 6. The Effects of Wash Buffer for CRISPR/Cas12a Activity

The CRISPR/Cas12a reaction mixture was prepared as described at Method 1. Then, 100 μL prepared CRISPR/Cas12a reaction mixture was activated by adding 5 μL 1 μM triggering ssDNA. The fluorescence signal was measured continuously by iD3 plate reader (Molecular Devices, LLC.) with Ex=570 nm and Em=615 nm. Afterwards, 5 μL wash buffer from the Abcam IFN-γ ELISA kit was directly mixed into the activated CRISPR/Cas12a reaction mixture at 15 mins reaction time, and the fluorescence signal was continuously measured. 5 μL 1×PBS solution was also added as the control to evaluate the signal changes.

7. Optimization of Anti-HRP ssDNA-Abs Conjugate for CES-Amplifier

100 μL HRP labelled antibody from the commercial ELISA kit was added on the high-binding 96-well plate with the recommended concentration of the ELISA kit instruction and incubated at room temperature for 1 h. After 3 times 1×PBS wash, 100 μL prepared anti-HRP ssDNA-Abs conjugate with different concentrations (0, 0.625, 1.25, 2.5, 5, 10 μg/mL) was applied at room temperature for 1 h. Afterwards, 3 times 1×PBS wash was applied and followed by applying 100 μL CRISPR/Cas12a reaction mixture and incubated at room temperature for 1 h. The fluorescence signal was then measured by iD3 plate reader (Molecular Devices, LLC.) with Ex=570 nm and Em=615 nm.

### 8. Standard Protocol for CES-Amplifier Applying to Commercial ELISA Kit

Firstly, the protocol of the commercial ELISA kit was followed until the analyte-antibody sandwich structure has been formed with the use of HRP-labelled detection antibody. Then, instead of applying TMB colorimetric substrates as the last step, 3 times wash of 350 μL PBS with 0.5 mg/mL BSA has been applied. Afterwards, 100 μL 10 μg/mL prepared anti-HRP ssDNA-Abs conjugate was applied and incubated at room temperature for 1 h. Then, after 3 times wash of 350 μL PBS with 10 mg/mL BSA, 100 μL prepared CRISPR/Cas12a reaction mixture was applied and incubated at room temperature for 1 h. The fluorescence signal was measured by iD3 plate reader (Molecular Devices, LLC.) with Ex=570 nm and Em=615 nm.

### Method S1

After the CRISPR/Cas12a reaction mixture has been prepared as described in Method 1, For verifying the CRISPR/Cas12a collateral cleavage, 5 μL of each triggering ssDNA (5′-bio, 5′-bio and 3′-TR, 3′-TR and no modification) was mixed with 95 μL of prepared CRISPR/Cas12a reaction mixture. The reaction was set at room temperature for an endpoint fluorescence signal intensity measurement at 60 mins by iD3 plate reader (Molecular Devices, LLC.) at Excitation wavelength (Ex)=570 nm, Emission wavelength (Em)=615 nm. CRISPR/Cas12a reaction mixture with no triggering ssDNA added has been used as negative control (Neg).

### Method S2

After the CRISPR/Cas12a reaction mixture has been prepared as described in Method 1, For verifying the CRISPR/Cas12a collateral cleavage, 5 μL of 5′-bio triggering ssDNA was mixed with 95 μL of prepared CRISPR/Cas12a reaction mixture. The reaction was set at room temperature for a dynamic fluorescence signal intensity measurement by iD3 plate reader (Molecular Devices, LLC.) at Excitation wavelength (Ex)=570 nm, Emission wavelength (Em)=615 nm for every 10 mins. 5 μL of PBS has been mixed with 95 μL of CRISPR/Cas12a reaction mixture as negative control (0).

### Method S3

After the CRISPR/Cas12a reaction mixture has been prepared as described in Method 1, 5 μL of each concentrations of prepared ssDNA-Abs conjugate (13.33, 6.67, 3.33, 1.67, 0 μg/mL) was mixed with 95 μL of prepared CRISPR/Cas12a reaction mixture.

The reaction was set at room temperature for a dynamic fluorescence signal intensity measurement by iD3 plate reader (Molecular Devices, LLC.) at Excitation wavelength (Ex)=570 nm, Emission wavelength (Em)=615 nm for every 10 mins. 5 μL of PBS has been mixed with 95 μL of CRISPR/Cas12a reaction mixture as negative control.

### Method S4

After the CRISPR/Cas12a reaction mixture has been prepared as described in Method 1, 5 μL of each prepared ssDNA-Abs conjugate (goat anti-HRP, anti-mouse, anti-rabbit and anti-IFN-γ antibodies) was mixed with 95 μL of prepared CRISPR/Cas12a reaction mixture. The reaction was set at room temperature for an endpoint fluorescence signal intensity measurement at 60 mins by iD3 plate reader (Molecular Devices, LLC.) at Excitation wavelength (Ex)=570 nm, Emission wavelength (Em)=615 nm. CRISPR/Cas12a reaction mixture with no ssDNA-Abs conjugate added has been used as negative control (Neg−).

### Method S5

100 μL HRP labelled antibody from the commercial ELISA kit was added on the high-binding 96-well plate with the recommended concentration of the ELISA kit instruction and incubated at room temperature for 1 h. After 3 times 1×PBS wash, 100 μL prepared rabbit anti-HRP ssDNA-Abs conjugate was applied at room temperature for 1 h. Afterwards, 3 times 1×PBS wash was applied and followed by applying 100 μL CRISPR/Cas12a reaction mixture and incubated at room temperature for 1 h. The fluorescence signal was then measured by iD3 plate reader (Molecular Devices, LLC.) with Ex=570 nm and Em=615 nm.

### Method S6

Firstly, the protocol of the commercial ELISA kit was followed until the analyte-antibody sandwich structure has been formed with the use of HRP-labelled detection antibody. Then, instead of applying TMB colorimetric substrates as the last step, 3 times of 1×PBS wash has been applied. Afterwards, 100 μL different concentrations of prepared anti-HRP ssDNA-Abs conjugate (0, 1, 2.5, 5, 10 μg/mL) was applied and incubated at room temperature for 1 h. Then, after 3 times 1×PBS wash, 100 μL prepared CRISPR/Cas12a reaction mixture was applied and incubated at room temperature for 1 h. The fluorescence signal was measured by iD3 plate reader (Molecular Devices, LLC.) with Ex=570 nm and Em=615 nm.

### Method S7

The Abs-ssDNA conjugate was firstly diluted in either ELISA kit antibody diluent buffer (A) or PBS (C) at room temperature for 15 mins. Two buffers with no ssDNA-Abs conjugate added were used as controls. Then, 5 μL of each treated Abs-ssDNA conjugate solution or controls was mixture with 95 μL prepared CRISPR/Cas12a mixture. The reaction was set at room temperature for a dynamic fluorescence signal intensity measurement by iD3 plate reader (Molecular Devices, LLC.) at Excitation wavelength (Ex)=570 nm, Emission wavelength (Em)=615 nm.

### Method S8

Firstly, the protocol of the commercial ELISA kit was followed until the analyte-antibody sandwich structure has been formed with the use of HRP-labelled detection antibody. Then, in comparison to directly add 100 μL TMB colorimetric reagent solution from the ELISA kit following the instruction, an additional 3 times of 1×PBS wash has also been applied, and then, 100 μL TMB colorimetric reagent solution from the ELISA kit has been added. After the reactions have been developed at room temperature for 15 mins under dark. The absorbance has been measured by iD3 plate reader (Molecular Devices, LLC.) at 440 nM.

### Results

### 1. Schematics of the CRISPR/Cas12a-Based ELISA Sensitivity Amplifier

Bringing together the highly efficient collateral cleavage activity of CRISPR/Cas12a with the conventional sandwich immunoassay format forms the central idea in the CES-Amplifier. This has been realized by the synthesis of a single strand DNA (ssDNA) oligo and anti-HRP antibody (ssDNA-Abs) conjugate. This bi-functional ssDNA-Abs conjugate needs to retain sufficient specific target recognition function as the original antibody, and it also must play a role of the nucleic acid target for the CRISPR/Cas12a system. This ssDNA-Abs conjugate can directly activate the downstream CRISPR/Cas12a ribonucleoprotein (RNP). This enables signal transduction and amplification without the need for intermediate nucleic acid conversion and synthesis, which can lead to a complex system design and its reduced reliability. Each of the ssDNA oligos on the conjugate has a complementary nucleic acid sequence to the guide RNA of the CRISPR/Cas12a RNP which can trigger collateral cleavage of the Cas12a nuclease. The resulting degradation of the separately introduced ssDNA-linked fluorescence quenched probe generates highly amplified fluorescent signal for final detection. The number of activated CRISPR/Cas12a RNPs is correlated with the number of triggering ssDNA molecules, hence, the amplified fluorescence signal can quantitatively represent the presence of the formed analyte-antibody sandwich structure on the ELISA plate.

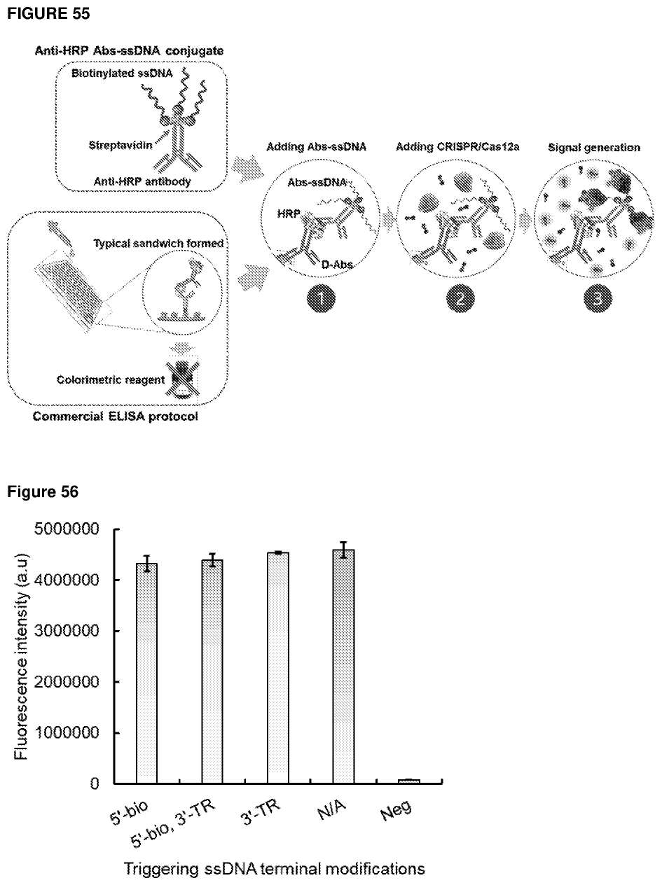

The schematic of the CES-Amplifier is shown in FIG. 55. To produce the final amplified signal from the CES-Amplifier, three simple steps are added directly to the ELISA workflow after the immune-sandwich structure has been formed according to the commercial ELISA kit instruction. These steps are: (1)—introducing the anti-HRP ssDNA-Abs conjugate, instead of adding the conventional colorimetric substrates, such as the 3,3′,5,5′-Tetramethylbenzidine (TMB) (this conjugate recognizes and binds to the HRP enzyme on the detection antibody of the sandwich structure); (2)—applying the CRISPR/Cas12a RNP together with standard quenched fluorescent DNA probes; (3)—directly measuring the amplified fluorescent signal on the ELISA plate. Thus, the ELISA amplifier represents an add-on component to the original ELISA assay which significantly increases the detection sensitivity, without changing the original ELISA protocol and with limited additional costs.

### 2. Verifying Single Strand DNA Induced CRISPR/Cas12a Activation

As the triggering ssDNA longer than 25 nt has shown optimal trans-cleavage activation efficiency for CRISPR/Cas12a RNP with gRNA spacer length>20 nt, a 30 nt triggering ssDNA and its corresponding gRNA with 21 nt spacer sequence were used in this study. Prior to the synthesis of the ssDNA-Abs conjugate, the 30 nt triggering ssDNA oligonucleotide has been directly synthesized (Sangon Ltd.), with biotin and Texas red terminal modifications as shown in Table 10. We found that all our triggering ssDNA oligos can successfully activate the corresponding CRISPR/Cas12a RNP and set off the collateral cleavage activity (FIG. 56). The results shown no significant difference in activation efficiencies between different types of triggering ssDNA oligos (FIG. 56). Therefore, the terminal modifications of ssDNA oligos have not compromised the CRISPR/Cas12a activation and signal amplification efficiency.

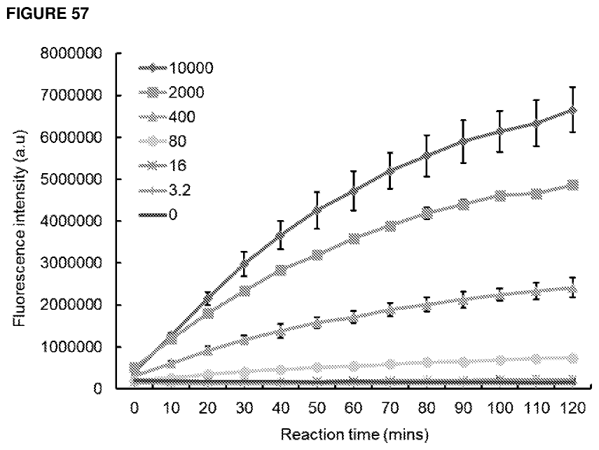

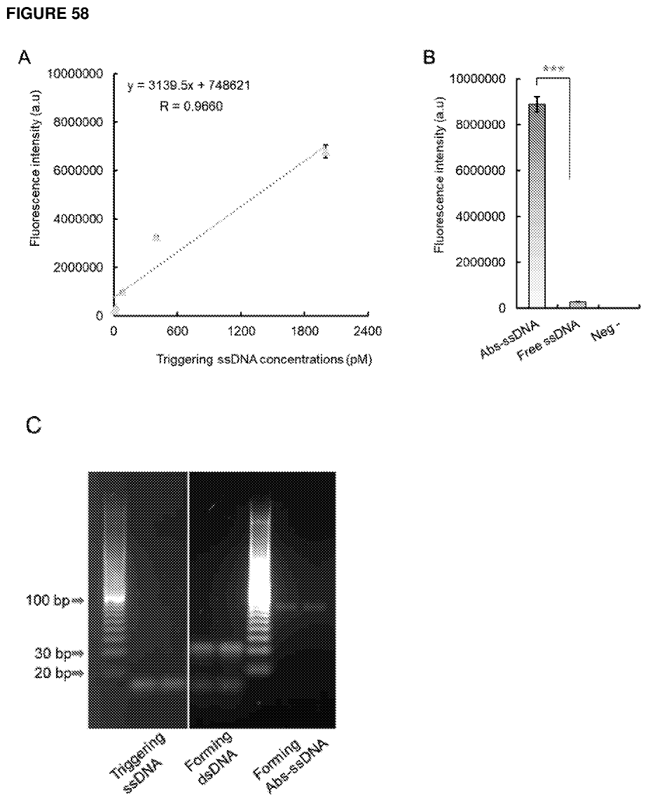

Furthermore, the activation efficiency of CRISPR/Cas12a for different concentrations of biotin labelled triggering ssDNA (3.2 pM to 10 nM) has also been investigated. Triggering ssDNA with concentrations higher than 16 pM has led to a significantly activation of the CRISPR/Cas12a within 30 mins, and the fluorescence intensity continued to increase at longer reaction times for all concentrations (FIG. 57). The results also indicated that higher concentration of ssDNA could lead to a higher activation level of Cas12a, and a positive correlation between fluorescence intensity and triggering ssDNA concentration has been observed at 1 hour reaction time (FIG. 58A, 57). This demonstrates the potential of quantitative response of a ssDNA oligo activated CRISPR/Cas12a and hence signal amplification.

3. Formation of the Anti-HRP ssDNA-Abs Conjugate

To synthesise the ani-HRP ssDNA-Abs conjugate, an anti-HRP antibody was firstly conjugated with streptavidin by directly using a commercial conjugation kit (Abcam), then the biotinylated triggering ssDNA (with or without 3′-Texas Red labelling) was directly mixed with the streptavidin conjugated anti-HRP antibody to form the anti-HRP ssDNA-Abs conjugate. The formed anti-HRP ssDNA-Abs conjugate was then verified by remove of the unbound triggering ssDNA (5′ biotin and 3′ Texas Red labelled) with a centrifugation-based filtering using 100 kD low-binding PES membrane. The remaining fluorescence signal indicated the successful binding of the triggering ssDNA to the anti-HRP antibody to form the ssDNA-Abs conjugate (FIG. 58B). Additionally, an electrophoretic mobility shift assay (EMSA) was applied to confirm the successful formation of this ssDNA-Abs conjugate. As the total molecular weight was significantly increased for the ssDNA conjugated onto the antibody, the movement of the formed ssDNA-Abs conjugate in the gel was significantly slower when comparing to either the free triggering ssDNA or the dsDNA formed by binding to its complementary sequence (FIG. 58C).

4. Anti-HRP ssDNA-Abs Conjugate for CRISPR/Cas12a Signal Amplification

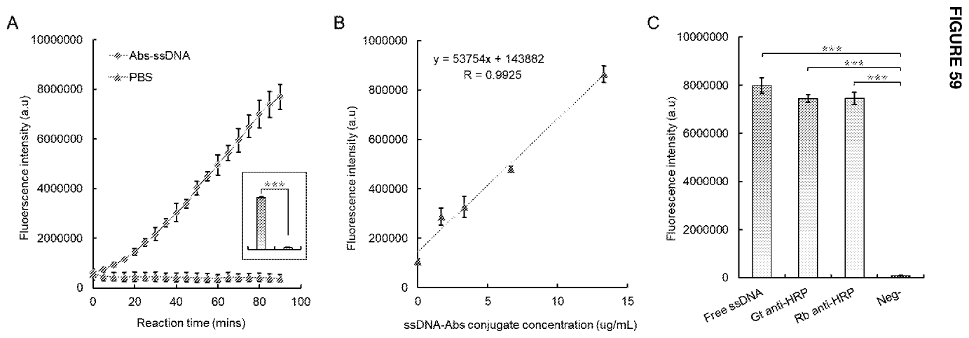

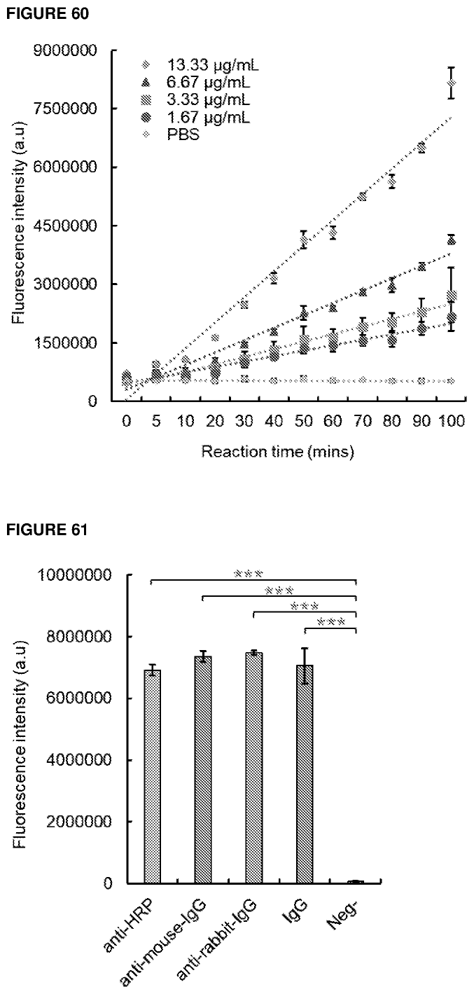

After the anti-HRP ssDNA-Abs conjugate has been synthesized, the functionality of thus prepared ssDNA-Abs conjugate to activate CRISPR/Cas12a RNP was tested. After triggering the CRISPR/Cas12a system by the synthesized anti-HRP ssDNA-Abs conjugate at room temperature, we found that, compared to the negative control, the synthesized anti-HRP ssDNA-Abs conjugates could successfully activate the CRISPR/Cas12a and generate highly amplified fluorescence signals, and the fluorescence intensity continued to increase with the reaction time increased in isothermal conditions, here at room temperature (FIG. 59A). More importantly, when different concentrations of the anti-HRP ssDNA-Abs conjugate (0, 1.67, 3.33, 6.67, 13.33 μg/mL) have been applied for CRISPR/Cas12a activation, all the concentrations have successfully led to the activation of CRISPR/Cas12a RNP and generated the continuously increased fluorescence signal during the reaction (FIG. 60). The conjugate concentration was tightly correlated with the fluorescence intensity at 1 hour reaction time at room temperature, which indicated the potential of CRISPR/Cas12a collateral cleavage in quantitatively representing the presence of anti-HRP ssDNA-Abs conjugate under isothermal condition (FIG. 59B).

In addition, the activation efficiency changes after conjugated onto anti-HRP antibody have also been evaluated in comparison to free triggering ssDNA. The results indicated that a similar activation pattern has been observed, and no significant fluorescence intensity change has been observed between ssDNA-Abs conjugate to free triggering ssDNA, which indicates an uncompromised CRISPR/Cas12a activation capability for our synthesized anti-HRP ssDNA-Abs conjugates (FIG. 59C). Also, ssDNA-Abs conjugate with different anti-HRP antibody sources (goat anti-HRP and rabbit anti-HRP) has shown no significant difference in CRISPR/Cas12a activation efficiency (FIG. 59C). Furthermore, we also investigated the changes of activation efficiency by changing the antibody, which have shown that, with our DNA-protein conjugate strategy, the type of antibody has not caused an obvious negative effect to the CRISPR/Cas12a's triggering ssDNA recognition process (FIG. 61).

5. Target Binding Affinity of the Anti-HRP ssDNA-Abs Conjugate

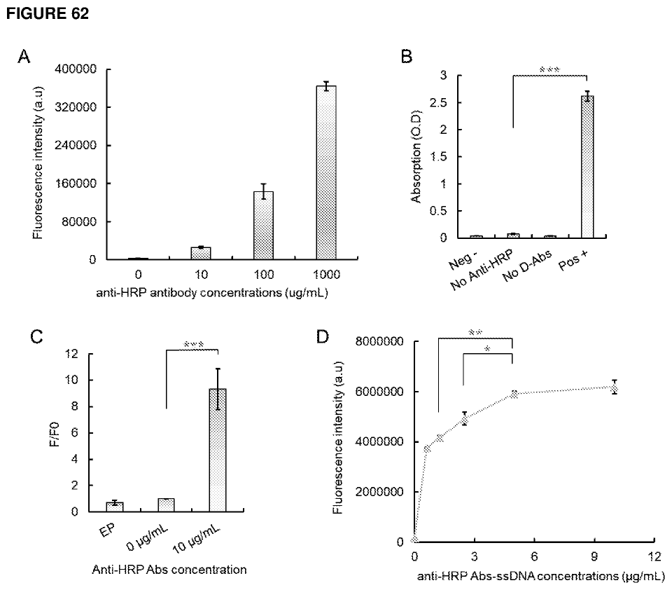

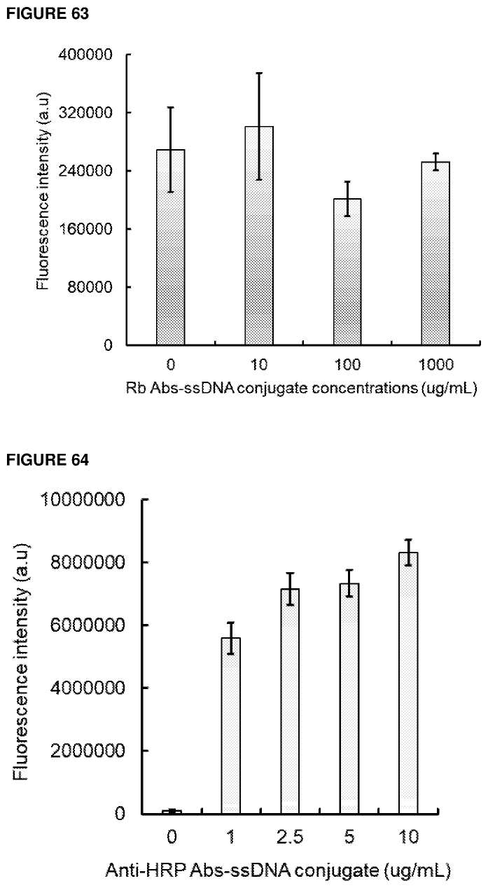

Before applying the anti-HRP ssDNA-Abs conjugate, the original HRP affinity of the anti-HRP antibodies has firstly been verified by using the detection antibody from a commercial ELISA kit (Abcam, ab174443), which was labelled with the HRP enzyme. The results shown that the binding ability of anti-HRP antibodies can vary significantly depending on different antibody sources, and, in our test, only goat anti-HRP antibody exhibited a quantitative response to the HRP-labelled detection antibody (FIG. 62A, 63). Therefore, goat anti-HRP ssDNA-Abs has been selected for our demonstration of CES-Amplifier in this study. We emphasise that the antibody source might be different in other commercial ELISA kits, so future users of our CES-Amplifier will need to conduct this test themselves for their selected kit.

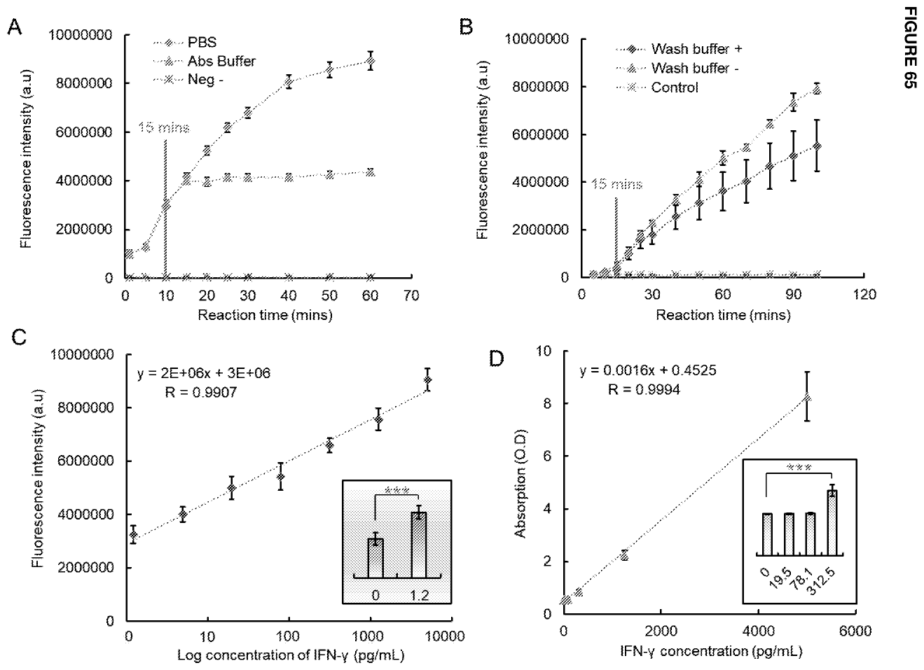

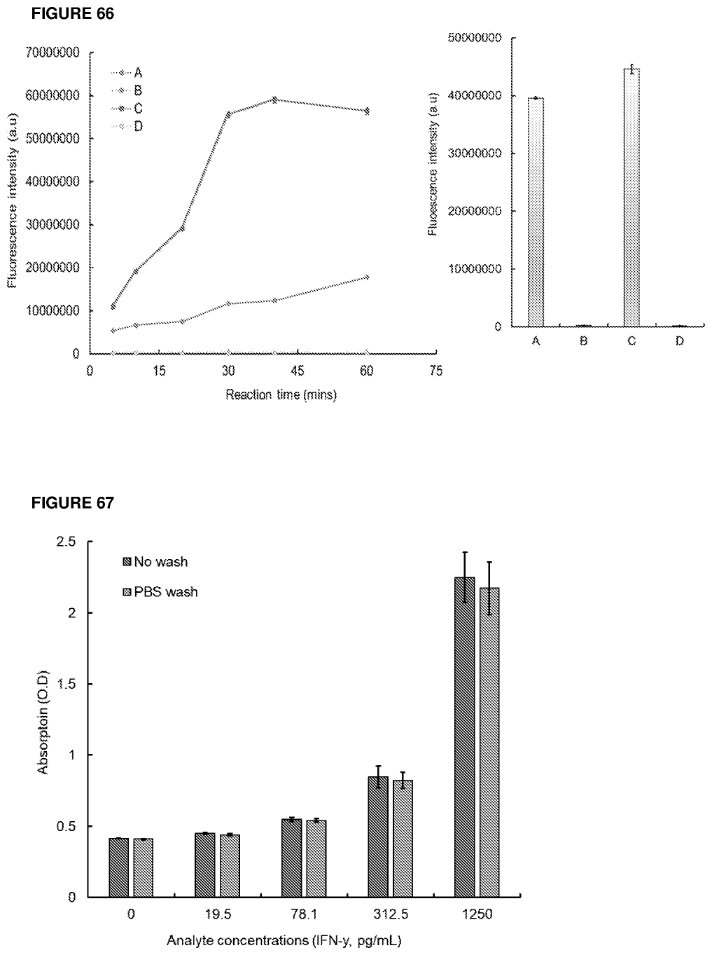

The target binding affinity of our prepared goat anti-HRP ssDNA-Abs conjugate to the ELISA detection antibody has also been tested. To this aim, after attaching the prepared anti-HRP ssDNA-Abs conjugates onto the surface of a high-binding 96-well plate, the detection antibody from the ELISA kit has been added. After removing the unbound detection antibody, the TMB colorimetric substrate has been introduced. Compared with the surface without anti-HRP ssDNA-Abs conjugate, significantly increased absorption levels (P<0.001) revealed the successful capture of the HRP-labelled detection antibody (FIG. 62B). Afterwards, the ELISA detection antibody has firstly been fixed onto the 96-well plate surface, and our CES-Amplifier scheme has been applied with the same anti-HRP ssDNA-Abs conjugate and the CRISPR/Cas12a reaction mixture (Methods 1). In the presence of the HRP-labelled detection antibody, a significantly increased fluorescent intensity (P<0.001) confirmed the binding affinity of our anti-HRP ssDNA-Abs conjugate, and demonstrated the anticipated function of our CES-Amplifier in the presence of the HRP-labelled detection antibody from the ELISA kit (FIG. 62C). With the ssDNA-Abs conjugate concentrations increased (0, 0.625, 1.25, 2.5, 5, 10 μg/mL), an increased final fluorescence signal has also been observed which indicated the quantitative recognition of the HRP-labelled detection antibody, and started to saturate after concentration of 5 μg/mL (FIG. 62D). This saturated fluorescence signal indicated the excess amount of ssDNA-Abs conjugate compared to existed HRP epitopes. We also found that when the anti-HRP ssDNA-Abs conjugate was applied to recognize the HRP-labelled antibody on a formed antibody-sandwich structure, the fluorescence intensity continued to increase for ssDNA-Abs conjugate concentrations higher than 5 μg/mL (FIG. 64). This may indicate the potential loss of anti-HRP ssDNA-Abs conjugate due to binding to an unstable sandwich structure. To compensate for this effect, a relatively high anti-HRP ssDNA-Abs conjugate concentration (10 μg/mL) was selected for our CES-Amplifier.

### 6. Interactions Between CRISPR/Cas12a and the Original Components of Commercial ELISA Kits

Applying our CES-Amplifier directly to a commercial ELISA kit will lead to an inevitable contact of the components from the commercial ELISA kit with the CES-Amplifier components, which may lead to potentially hostile environments for the CRISPR/Cas signal amplification. We have therefore assessed these interactions. We found that when the (undisclosed) antibody dilution buffer from the ELISA kit has been introduced into our CRISPR/Cas12a reaction mixture, a significant suppression activity for the CRISPR/Cas12a collateral cleavage has been observed even when the antibody dilution buffer has been diluted 20 times (FIG. 65A, 66). To address this, after the antibody-analyte sandwich structure has been formed following the ELISA kit instruction, we incorporated an additional washing step to remove the remaining antibody dilution buffer from the 96-well plate before applying our CES-Amplifier scheme. We also found that the original ELISA wash buffer also had a noticeable negative effect on our CRISPR/Cas12a reaction system even under 20 times dilution. This buffer led to a significantly reduced fluorescence intensity, and increased variability of the fluorescence signals suggesting an underpinning destabilization of the activated CRISPR/Cas12a RNP and/or the triggering ssDNA complex (FIG. 65B). Therefore, 1×PBS buffer has been selected as a replacement for the additional washing step. We found that the stability of the formed sandwich structure has not been affected significantly under the additional 1×PBS wash (FIG. 67). Therefore, our choice of 1×PBS buffer can be safely applied to the ELISA kit directly as the additional wash buffer, and it will not jeopardise the formed sandwich structure.

### 7. Performance of the CES-Amplifier for the Commercial ELISA Kit

To demonstrate the effectiveness of our CES-Amplifier to increase the system sensitivity of a commercial ELISA kit, we used IFN-γ ELISA kit (Abcam, ab174443) as an example. Its quoted sensitivity is 470 pg/mL which insufficiently high for the range of IFN-γ concentrations in physiological conditions (in normal human blood the baseline of IFN-γ is between 17 to 105 pg/mL, and the IFN-γ concentration in plasma from an example disease (Dengue) can be less than 100 pg/L.)

After all these preparations, our CES-Amplifier scheme has been integrated into the ELISA kit at its final step, where the antibody-analyte sandwich structure has been formed. Instead of using the original TMB substrate, our anti-HRP ssDNA-Abs conjugate has been directly introduced to recognize the HRP-labelled detection antibody on the sandwich structure. With 1-hour incubation at room temperature and removal of the unbound anti-HRP ssDNA-Abs conjugate, the downstream CRISPR/Cas12a system has been added for signal generation. We tested the entire system in the standard blood plasma diluent (provided in the ELISA kit) spiked with IFN-γ. After the integration of our CES-Amplifier to the commercial ELISA kit, a detection linear range from 1.2 pg/mL to 5000 pg/mL has been observed with linear correlation R=0.9907, and a 1.2 pg/mL sensitivity based on the significantly higher fluorescent signal comparing to the negative control (P<0.001) (FIG. 65C). For comparison, the IFN-γ detection with the original ELISA kit protocol under same experimental conditions, produced the value of 312.5 pg/mL for the kit sensitivity (FIG. 65D), which is in close agreement with the sensitivity of 470 pg/mL claimed in this kit instruction. In contrast, our CES-Amplifier successfully realized 2 orders of sensitivity increase. Also, the 3 orders of magnitudes log linear range (1.2 pg/mL to 5 ng/mL) achieved by CES-Amplifier is 1 order of magnitude wider than 2 orders of magnitude range (0.468 ng/mL to 30 ng/mL) claimed in our kit at lower concentration level. This expansion can be helpful in evaluation of IFN-γ under significant variations.

By using the anti-HRP antibody, the CES-Amplifier has been directly applied to a commercial ELISA kit for IFN-γ detection without modifying its original reagents or protocol. Comparing to its original ELISA assay performance, applying the CES-Amplifier resulted in over two orders of magnitude of sensitivity increase from the original sensitivity of 312.5 pg/mL to 1.2 pg/mL, along with 1 order of magnitude increase in detection range. This is important e.g. for comprehensive evaluation of IFN-γ changes under certain physiological conditions such as in blood where IFN-γ levels can vary from 17 pg/mL to 1500 pg/mL, which is below the originally claimed detection limits of the commercial ELISA kits. More importantly, without the need for full control of ingredients in unknown immunoassays, but, instead, by simply replacing the original kit buffer system with our own controlled reaction environment after one additional washing, our anti-HRP-based CES-Amplifier approach has the potential to be directly applied, as a stand-alone addition, to a wide range of other commercial ELISA kits which use the HRP-conjugated antibody. Under such circumstances, our CES-Amplifier provides a deliverable, user-friendly and affordable biosensing innovation for the end-users, and also represents a significant advance towards further integration of the CRISPR/Cas technology into the mainstream biosensing applications for the end-users.

### Example 8. Colorimetric Reporter (MB-ssDNA-HRP) for CRISPR/Cas12a Biosensing System

This Example is directed to use of a novel colorimetric reporter for use in a CRISPR/Cas12a biosensing system. The novel HRP reporter was successfully synthesized using magnetic beads (MB), ssDNA linker, and HRP.

### Materials and Methods

### 1. Materials and Reagents

Biological reagents: EnGen® Lba Cas12a (Cpf1) protein (New England Biolab), 10×NEB 2.1 buffer (New England Biolab), phosphate buffered saline (PBS) (Sigma, 10 mM, pH=7.4), Dithiothreitol (DTT) (Sigma, DTT-RO), SigmaFast OPD (P9187), and Magnetic beads (Spherotech, SVM-08-10).

Oligos: All designed RNA and DNA oligos were synthesized by Sangon Biotech.

TABLE 11
RNA and DNA oligo sequences used in this study.

| | Oligo | | Length | Modifi- |
|---|---|---|---|---|
| Name | type | Sequence 5′-3′ | (nt/bp) | cation |
| Guiding | ssRNA | UAA UUU CUA | 42 | N/A |
| RNA | | CUA AGU GUA | | |
| (gRNA) | | GAU GGG GGG | | |
| | | GGU UGG UAG | | |
| | | GGU GUC | | |
| | | (SEQ ID No. 6) | | |
| Target | ssDNA | GAA GAC ACC | 24 | N/A |
| DNA | | CTA CCA ACC | | |
| | | CCC CCC | | |
| | | (SEQ ID No. 5) | | |
| ssDNA | ssDNA | TTATTTTTTTTATT | 20 | 3′-Biotin; |
| linker | | TTTTAT | | 5′-FAM |
| | | (SEQ ID No. 4) | | |

### Methods

### 2. Synthesis and Optimization of Magnetic Beads-ssDNA-HRP Reporter.

Firstly, 20 μL 0.5 w/v streptavidin modified magnetic beads (0.74 μm) was washed twice with PBS buffer. Subsequently, 100 μL of a range of biotin-ssDNA-FAM solution (0, 0.5, 1, 2, 4 μM ssDNA in 1% BSA solution) was mixed with magnetic beads for 30 min at room temperature, free ssDNA was removed by PBS wash. The fluorescence signal was tested using ID3 plate reader (Excitation wavelength of 480 nm and emission wavelength of 520 nm). After formation of the magnetic beads-ssDNA conjugate, HRP labelled anti-FAM antibody was introduced to form the magnetic beads-ssDNA-HRP conjugation. A range of anti-FAM antibody concentration was tested (0, 1.25, 2.5, 5, 10, 20 μg/mL), and the free antibody was removed by PBS wash. The final HRP reporter was added in 100 μL OPD solution. After incubation for 10 min, the absorbance was detected using ID3 plate reader at 492 nm.

### 3. Validation of Synthesized Magnetic Beads-ssDNA-HRP Reporter.

A range of HRP reporter (0, 12.5, 25, 50, 100 μg/mL) was applied in 100 μL OPD solution. After incubation for 10 min, the absorbance was detected using ID3 plate reader at 492 nm.

### 4. Optimization of Magnetic Beads-ssDNA-HRP Reporter Concentration in CRISPR/Cas12a Mixture.

CRISPR/Cas12a reaction mixture was prepared as follow: 10 μL 10 μM (100 pmol) of Cas12a protein was gently mixed with 5 μL 20 μM (100 μmol) gRNA and 36 μL 1 M DTT in a total of 3.6 mL 1×NEB 2.1 buffer. Subsequently, a range of HRP reporter was added (0, 25, 50, 100, 200, 400 μg/mL) and well mixed to form the final reaction mixture.

5 μL 1 μM target DNA was applied in 100 μL prepared CRISPR/Cas reaction mixture for starting the CRISPR reaction process. After incubation for 60 min at 37° C., magnetic separation was applied, and 50 μL supernatant was taken and added into 100 μL OPD solution. After incubation for 10 min, the absorbance was detected using ID3 plate reader at 492 nm.

### 5. Application of HRP Reporter Based CRISPR/Cas12a Biosensing System for the Detection of Target Nucleic Acid

CRISPR/Cas12a reaction mixture was prepared as follow: 10 μL 10 μM (100 μmol) of Cas12a protein was gently mixed with 5 μL 20 μM (100 μmol) gRNA, 36 μL 1 M DTT and 72 μL 1% HRP beads reporter in a total of 3.6 mL 1×NEB 2.1 buffer. A range of target DNA (0, 0.1 nM, 1 nM, 10 nM, 100 nM, 1 μM) was applied in 100 μL prepared CRISPR/Cas reaction mixture for starting the CRISPR reaction process. After incubation for 60 min at 37° C., magnetic separation was applied, and the supernatant was removed. Subsequently, 100 μL OPD solution was added, and incubated for 10 min. The absorbance was detected using ID3 plate reader at 492 nm.

### Results

### 1. Schematic of HPR Reporter Based CRISPR/Cas12a Biosensing System.

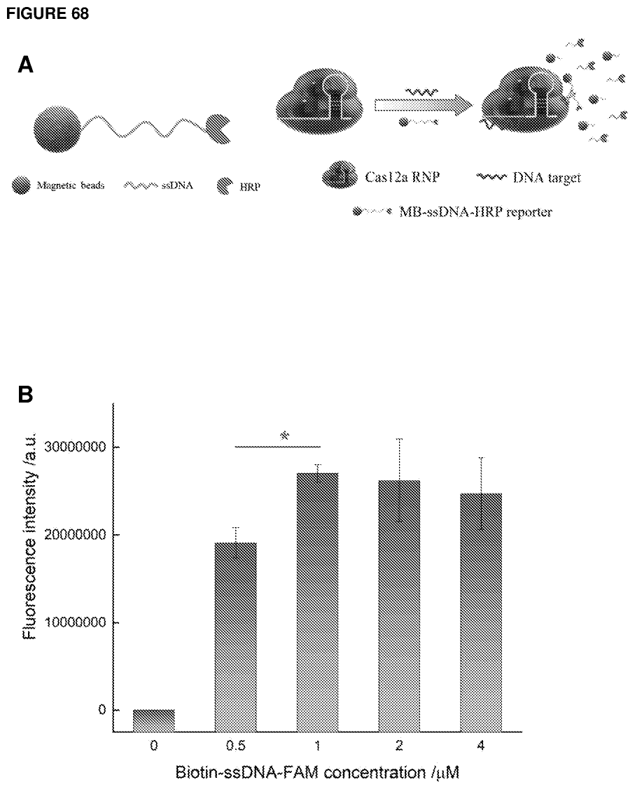

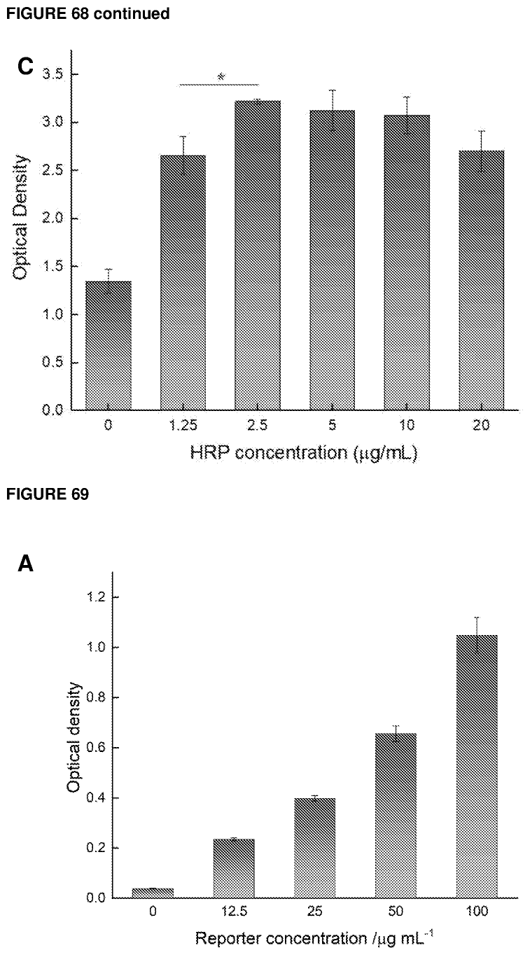

As Shown in FIG. 68 A), after Mixing DNA Target with Cas12a RNP, the Trans-cleavage ability of Cas12a RNP is activated, which non-specifically cleaves the ssDNA in the MB-ssDNA-HRP reporter to release the HRP protein. The remaining HRP on the MB-ssDNA-HRP reporter and the free HRP in the solution both can be applied in colorimetric substrate for signal readout.
2. The synthesis and optimization of magnetic beads-ssDNA-HRP reporter.

The magnetic beads-ssDNA-HRP reporter was synthesized by conjugation of ssDNA on magnetic beads following the attachment of HRP on ssDNA (FIG. 68A). Streptavidin modified magnetic beads were applied (0.1% w/v), and the concentration of biotin-ssDNA-FAM was optimized to be 1 μM (FIG. 68B), and the concentration of HRP labelled anti-FAM antibody was optimized to be 2.5 μg/mL (FIG. 68C).

### 3. Validation of Synthesized Magnetic Beads-ssDNA-HRP Reporter.

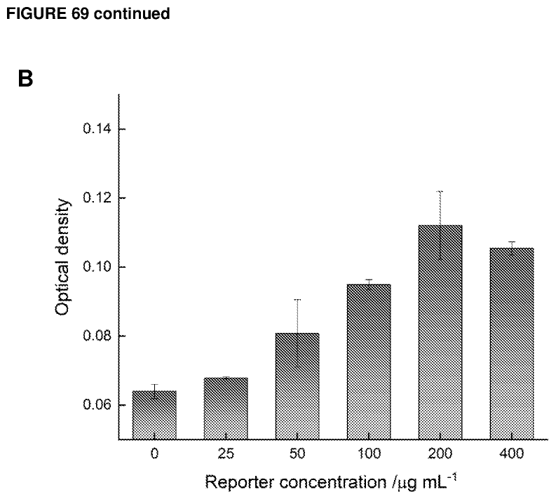

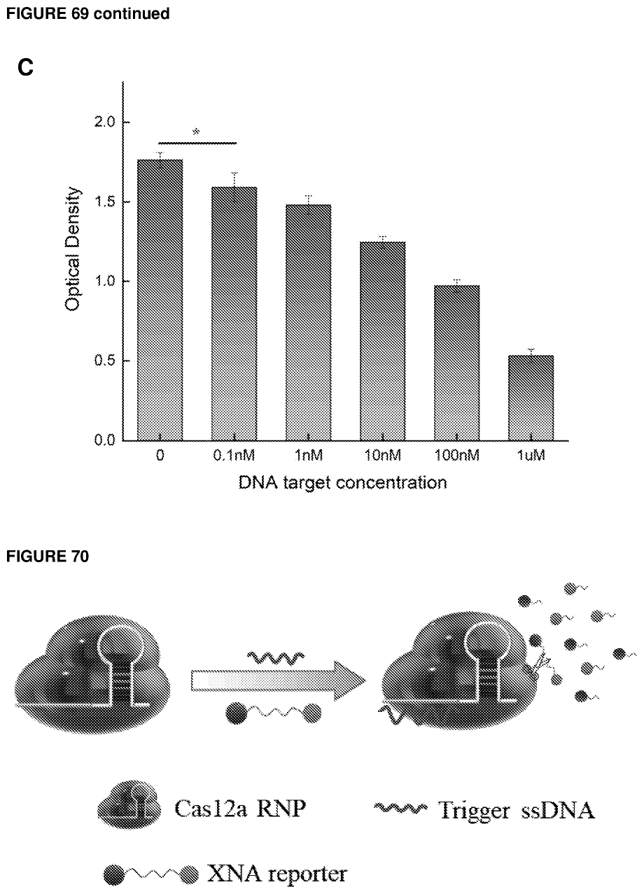

The enzymatic activity of HRP on the magnetic beads-ssDNA-HRP reporter was further validated by adding a variety of concentrations of magnetic beads-ssDNA-HRP reporter into OPD solution (FIG. 69A). The optical density increased with the increase of reporter concentration, demonstrating the feasible enzymatic activity of HRP on the magnetic beads-ssDNA-HRP reporter.

### 4. Optimization of Magnetic Beads-ssDNA-HRP Reporter Concentration in CRISPR/Cas12a Mixture.

The Validated Reporter was Added in the CRISPR/Cas12a Mixture to Form the Form the Final Reaction Mixture. And the Concentration of Magnetic Beads-ssDNA-HRP Reporter was optimized to be 200 μg/mL (FIG. 69B).
5. Application of HRP reporter based CRISPR/Cas12a biosensing system for the detection of target nucleic acid

The successfully established HRP reporter based CRISPR/Cas12a biosensing system was applied for the detection of target nucleic acid (FIG. 69C). After adding the target nucleic acid into the reaction mixture, the trans-cleavage activity of Cas12a RNP was activated to cleave the magnetic beads-ssDNA-HRP reporter. After 60 min incubation at 37° C., the remaining HRP on magnetic beads-ssDNA-HRP reporter was collected using magnetic separation, and 100 μL OPD solution added inside for colorimetric signal readout. As shown in FIG. 69C, the limit of detection was 0.1 nM with a 4-log detection range from 0.1 nM to 1 μM.

### Discussion

In this example, the inventors developed a novel colorimetric reporter for a CRISPR/Cas12a biosensing system. This colorimetric reporter was synthesized using magnetic beads (MB), ssDNA linker, and HRP. The validated HRP reporter was utilized in CRISPR/Cas12a biosensing system for the detection of synthesized DNA target, and the limit of detection was evaluated to be 0.1 nM with 4 log detection range (0.1 nM to 1 μM). Collectively, the novel colorimetric reporter provides an effective approach for the developing of colorimetric CRISPR/Cas biosensing system for the measurement of nucleic acid and non-nucleic acid targets.

### Example 9. Xeno Nucleic Acids (XNA) Reporters for CRISPR/Cas12 Biosensing System

In this Example, the inventors designed diverse types of XNA reporters for CRISPR/Cas12 biosensing system. These novel XNA reporters were specifically designed using XNA as the linker, and Texas red was modified on the 5′ end of XNA, and BHQ2 was modified on the 3′ end of XNA. Since the fluorescence of Texas red was quenched by BHQ2, the original XNA reporters had negligible background. The performance of these XNA reporters was evaluated in CRISPR/Cas12 biosensing system, and suitable XNA reporters were selected as alternatives of DNA reporter for the developing of CRISPR/Cas12 biosensing system for the measurement of nucleic acid targets.

### Materials and Methods

### 1. Materials and Reagents

Biological reagents: EnGen® Lba Cas12a (Cpf1) protein (New England Biolab), 10×NEB 2.1 buffer (New England Biolab), phosphate buffered saline (PBS) (Sigma, 10 mM, pH=7.4).

Oligos: All designed RNA and DNA oligos were synthesized by Sangon Biotech.

TABLE 12
RNA and DNA oligo sequences used in this study.

| | Oligo | | Length | |
|---|---|---|---|---|
| Name | type | Sequence 5′-3′ | (nt/bp) | Modification |
| Guiding RNA | ssRNA | UAA UUU CUA CUA | 42 | N/A |
| (gRNA) | | AGU GUA GAU GGG | | |
| | | GGG GGU UGG UAG | | |
| | | GGU GUC | | |
| | | (SEQ ID No. 6) | | |
| Trigger ssDNA | ssDNA | GAA GAC ACC CTA | 24 | N/A |
| | | CCA ACC CCC CCC | | |
| | | (SEQ ID No. 5) | | |
| DNA reporter | ssDNA | TTATT | 5 | 5′-Texas red; 3′- |
| | | | | BHQ2 |
| LNA reporter | ssDNA | TTATT | 5 | 5′-Texas red; 3′- |
| | | | | BHQ2; A-LNA |
| Phosphorothioate | ssDNA | TTATT | 5 | 5′-Texas red; 3′- |
| reporter | | | | BHQ2; |
| | | | | A-Phosphorothioate |
| beta-L-DNA | ssDNA | TTATT | 5 | 5′-Texas red; 3′- |
| reporter | | | | BHQ2; A-beta-L-DNA |
| 2′-O-Methyl | ssDNA | TTATT | 5 | 5′-Texas red; 3′- |
| reporter | | | | BHQ2; A-2′-O-Methyl |
| 2-methoxyethyl | ssDNA | TTATT | 5 | 5′-Texas red; 3′- |
| reporter | | | | BHQ2; A-2- |
| | | | | methoxyethyl |
| 2-Aminopurine | ssDNA | TTATT | 5 | 5′-Texas red; 3′- |
| reporter | | | | BHQ2; A-2- |
| | | | | Aminopurine |
| 5-Aza-2′- | ssDNA | TTATT | 5 | 5′-Texas red; 3′- |
| deoxycytidine | | | | BHQ2; A-5-Aza-2′- |
| reporter | | | | deoxycytidine |
| Azobenzene | ssDNA | TTATT | 5 | 5′-Texas red; 3′- |
| reporter | | | | BHQ2; A- |
| | | | | Azobenzene |
| RNA reporter | ssDNA | TTATT | 5 | 5′-Texas red; 3′- |
| | | | | BHQ2; A-rA |
| 2F-RNA reporter | ssDNA | TTATT | 5 | 5′-Texas red; 3′- |
| | | | | BHQ2; A-2F-RNA |
| deoxyUridine | ssDNA | TTATT | 5 | 5′-Texas red; 3′- |
| reporter | | | | BHQ2; A- |
| | | | | deoxyUridine |
| 5-Nitroindole | ssDNA | TTATT | 5 | 5′-Texas red; 3′- |
| reporter | | | | BHQ2; A-5- |
| | | | | Nitroindole |

### 2. Evaluation the Performances of XNA Reporters in CRISPR/Cas12 Biosensing System

CRISPR/Cas12a reaction mixture was prepared as follow: 10 μL 10 μM (100 pmol) of Cas12a protein was gently mixed with 5 μL 20 pM (100 μmol) gRNA and 36 μL 1 M DTT in a total of 3.6 mL 1×NEB 2.1 buffer. Then, 6 μL 100 pM (0.6 nmol) XNA linked fluorescent reporter was added and well mixed to form the final reaction mixture.

5 μL 1 pM trigger ssDNA was applied in 100 μL prepared CRISPR/Cas reaction mixture for starting the CRISPR reaction process. ID3 plate reader was applied for the detection of fluorescence readout with excitation wavelength of 570 nm and emission wavelength of 615 nm.

### Results

### 1. Schematic of XNA Reporter Based CRISPR/Cas12 Biosensing System.

As Shown in FIG. 70, after Mixing Trigger ssDNA with Cas12 RNP, the Trans-Cleavage ability of Cas12 RNP is activated, which non-specifically cleaves the XNA reporter to release the Texas red from BHQ2 quencher for fluorescence signal readout.
2. Evaluation the performances of XNA reporters in CRISPR/Cas12 biosensing system

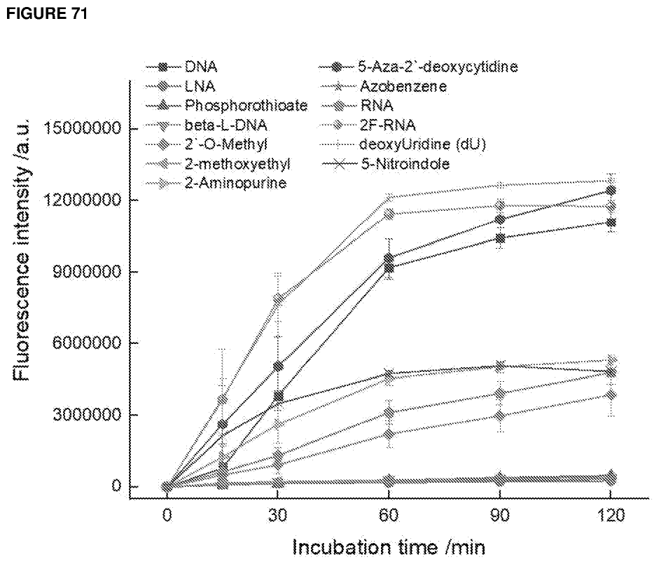

As shown in FIG. 71, all the reporters can be classified into three groups. Surprisingly, the first group of XNA reporters, including deoxyUridine reporter, 2F-RNA reporter, and 5-Aza-2′-deoxycytidine reporter, showed better or comparable performance as the DNA reporter. The second group of XNA reporters, including 5-Nitroindole reporter, 2-Aminopurine reporter, RNA reporter, and 2′-O-Methyl reporter, showed feasible performance and suitability for use as CRISPR/Cas12 reporters. The third group of remaining XNA reporters tested showed no obvious fluorescence change thus they cannot be utilized as CRISPR/Cas12 reporter. These XNA reporters include LNA reporter, Phosphorothioate reporter, beta-L-DNA reporter, 2-methoxyethyl reporter, and Azobenzene reporter.

### Discussion

In this research, the inventors designed and tested diverse types of XNA reporters for CRISPR/Cas12 biosensing applications. Among them, the deoxyUridine reporter, 2F-RNA reporter, and 5-Aza-2′-deoxycytidine reporter showed compatible and even enhanced performance compared with a DNA reporter, meaning that they can be utilized as alternative and superior options for the CRISPR/Cas12 biosensing system. The second group of XNA reporters shows feasible performance but still can be used as CRISPR/Cas12 reporters, including 5-Nitroindole reporter, 2-Aminopurine reporter, RNA reporter, and 2′-O-Methyl reporter. The remaining XNA reporters are not suitable for CRISPR/Cas12 biosensing systems. Collectively, the data suggests that specific XNA reporters represent alternative superior options for use in CRISPR/Cas biosensing systems for the measurement of nucleic acid and non-nucleic acid targets.

### Example 10. Demonstration of or Bi-Functional Antibody-Cas12a/gRNA (BAC) Conjugate for the Detection of Other Analytes

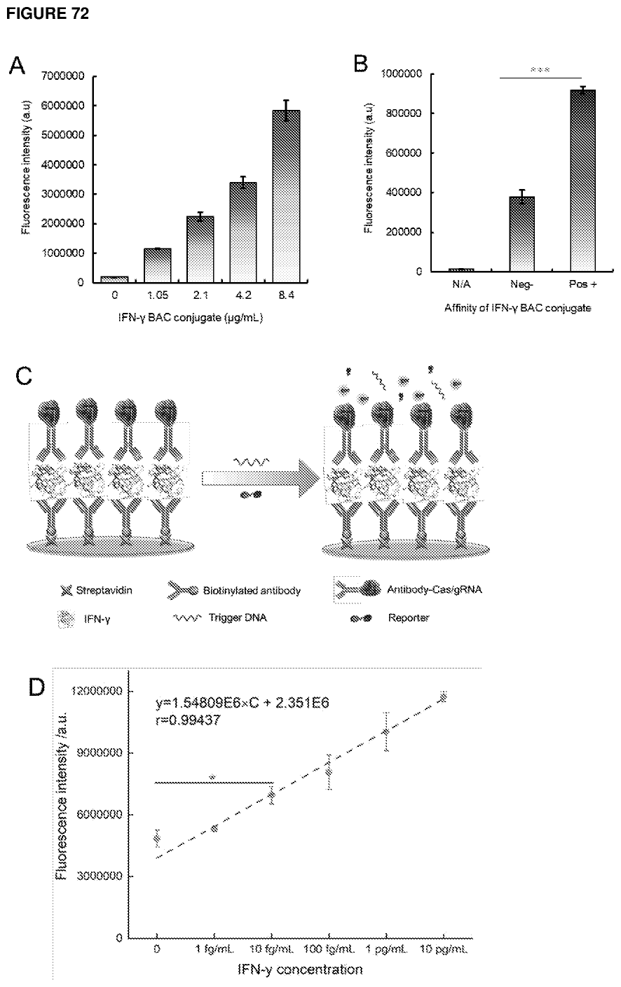

In addition to the successful implementation of the BAC conjugate in a typical immunoassay scheme with remarkable sensitivity ensuring single microorganism detection, demonstrated in in Example 4 above, the integration of the CRISPR/Cas12a-based signal amplification into a conventional immunoassay for molecular analytes has also now been demonstrated. The inventors accomplished this by the detection of a small protein, the cytokine IFN-γ. The BAC conjugate was firstly prepared using the same conjugation protocol as described in example 4 above but using an anti-IFN-γ antibody. The CRISPR/Cas12a trans-cleavage activity of the prepared anti-IFN-γ BAC conjugate was then tested. The results show the expected positive correlation between the fluorescence signal and the concentration of BAC conjugate, from 1.05 to 8.4 pg/mL (FIG. 72A). The analyte recognition test of antibody also indicated that the anti-IFN-γ BAC conjugate was able to respond its target analyte (FIG. 72B).

With the thus demonstrated bi-functional properties, the anti-IFN-γ BAC conjugate was then, again, used as the detection antibody in a sandwich immunoassay scheme for IFN-γ detection (FIG. 72C). In contrast with, Crypto detection where the same antibody can be used as both capture and detection antibody due to the presence of multiple antigens on the Crypto oocyst surface (˜4 m), for detection of small protein molecule, IFN-γ, a suitable pair of antibodies (targeting different epitopes) was needed to form the sandwich assay structure. A polyclonal anti-IFN-γ antibody was used as the capture antibody to form the sensing interface, and the BAC conjugate made from a monoclonal anti-IFN-γ antibody was then used as a detection antibody (FIG. 72C). After the formation of sandwich structure, the CRISPR reaction mixture was added to trigger the trans-cleavage for downstream fluorescence signal detection. The results show the assay sensitivity reached 10 fg/mL, the concentration for which a significantly higher signal (P=0.004) has been observed than in the absence of the analyte. A strong linear correlation (r=0.9944) was found between the fluorescence signal and cytokine IFN-γ at concentration between 10 fg/mL and 10 pg/mL. This confirms that, by changing the antibody in the BAC conjugate, it is possible to detect alternative analytes through the well-established immunoassay systems.

### Discussion

Antibodies, as the basis of immunosensing, have been widely used in analytical chemistry and clinical diagnostic fields. Their extraordinary specificity and existing large pool of commercial antibodies underpin their widespread utilisation as recognition molecules. Antibody modifications, and, particularly, antibody conjugation, have further facilitated their expanding application for novel biosensor development, including in the recently introduced CRISPR/Cas12a nuclease system. The trans-cleavage activity of CRISPR/Cas12a RNP offers high level of fluorescence signal amplification, and it has been engineered to be compatible with different immunoassay schemes. However, in currently reported CRISPR/Cas-associated immunosensing systems, the CRISPR/Cas12a RNP are merely used as a signal amplification step at the end of detection, which relies on the presence of additional nucleic acid molecules in the antibody-analyte recognition process to link it with the CRISPR/Cas12a trans-cleavage activity. These approaches are similar to the introduction additional nucleic acid amplification strategies, such as PCR, RPA, etc., in previous studies, which may lead to several potential issues, such as unintended degradation by nucleases inadvertently present in biological samples and non-specific amplification. The BAC conjugates described herein, for the first time directly brings together the CRISPR/Cas12a RNP with another recognition molecule (antibody), and successfully avoids the need for additional nucleic acid molecules during assay detection when the assay components are exposed to a biological sample.

The use of a commercially available conjugation kits to prepare the BAC conjugate will help end-users, including non-specialist users to apply the invention for a variety of different target binding constructs. Through the studies detailed herein use of a commercial conjugation kit does not significantly compromise the activities of the binding construct (e.g. antibody) or Cas12a/gRNA RNP.

Furthermore, with a simple change of the antibody for conjugating with the Cas12a/gRNA RNP, the inventors have demonstrated successful detection of two different types of targets, a whole microorganism and a small protein, reaching sensitivity of 1 single microorganism and 10 fg/mL of IFN-γ, respectively. Moreover, without requiring any changes to conventional immunoassay protocols/formats as detailed herein, the invention herein described is generally applicable for use in a variety of immunoassays for a broad range of different analytes to reach higher performance.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for the detection of a target in a sample, the method comprising:
(a) contacting the sample with:
(i) a first target binding construct;
(ii) a type V or type VI CRISPR/Cas effector protein;
(iii) a trigger nucleic acid sequence;
(v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
(iv) a labelled reporter construct, wherein said reporter construct comprises a nucleic acid that does not hybridize with the guide sequence of the guide RNA and is cleavable by the nuclease activity of the activated type V or type VI CRISPR/Cas effector protein; and
(b) measuring a detectable signal produced following cleavage of the labelled nucleic acid reporter by the type V or type VI CRISPR/Cas effector protein, thereby detecting the target in the sample;
wherein the type V or type VI CRISPR/Cas effector protein, the trigger nucleic acid sequence, the guide RNA, the type V or type VI CRISPR/Cas effector protein in combination with the guide RNA, or the type V or type VI CRISPR/Cas effector protein in combination with the guide RNA and the trigger nucleic acid is linked or conjugated to the first target binding construct to thereby co-locate the type V or type VI CRISPR/Cas effector protein and the target when present in the sample; and
wherein the target is not a nucleic acid sequence.

2. A method for the detection of a target in a sample, the method comprising:
(a) contacting the sample with:
(i) a first target binding construct;
(ii) a second target binding construct to immobilise or capture the target;
(iii) a type V or type VI CRISPR/Cas effector protein;
(iv) a trigger nucleic acid sequence;
(v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
(vi) a labelled reporter construct, wherein said reporter construct comprises a nucleic acid that does not hybridize with the guide sequence of the guide RNA and is cleavable by the nuclease activity of the activated type V or type VI CRISPR/Cas effector protein; and
(b) measuring a detectable signal produced following cleavage of the labelled nucleic acid reporter by the type V or type VI CRISPR/Cas effector protein, thereby detecting the immobilised or captured target;
wherein the type V or type VI CRISPR/Cas effector protein, the trigger nucleic acid sequence, the guide RNA, the type V or type VI CRISPR/Cas effector protein in combination with the guide RNA, or the type V or type VI CRISPR/Cas effector protein in combination with the guide RNA and the trigger nucleic acid is linked or conjugated to the first target binding construct to thereby co-locate the type V or type VI CRISPR/Cas effector protein and the target when present in the sample; and
wherein the target is not a nucleic acid sequence.

3. The method of claim 1 or 2, wherein the type V or type VI CRISPR/Cas effector protein is a Cas12a (Cpf1) or Cas12b (C2c1) protein, or Cas 13a or Cas 13b.

4. The method of any one of claims 1-3, wherein the reporter construct is a labelled RNA.

5. The method of any one of claims 1-3, wherein the reporter construct is a labelled DNA.

6. The method of any one of claims 1-5, wherein the reporter construct comprises at least one nucleotide containing a non-natural sugar.

7. The method of any one of claims 1-6, wherein the label on the reporter construct is an enzyme selected from horse radish peroxidase and alkaline phosphatase.

8. The method of claim 7, wherein the reporter construct is conjugated to a magnetic bead.

9. The method of any one of claims 1-7, wherein the trigger nucleic acid sequence is a single-stranded DNA sequence.

10. The method of any one of claims 1-9, wherein either or both of the first target binding construct and second binding constructs is an antibody or antigen binding fragment thereof.

11. The method of any one of claims 1-10, wherein the first or second target binding construct is immobilised on a substrate or conjugated to a magnetic bead.

12. The method of claim 11, further comprising the step of performing magnetic separation of the captured target bound to the first target binding construct from the sample when the first target binding construct is conjugated to a magnetic bead.

13. The method of any one of claims 1-12, wherein the first target binding construct is an antibody or antigen binding fragment thereof conjugated to said type V or type VI CRISPR/Cas effector protein.

14. The method of any one of claims 1-12, wherein the first target binding construct is an antibody or antigen binding fragment thereof conjugated to said guide RNA.

15. The method of any one of claims 1-12, wherein the first target binding construct is an antibody or antigen binding fragment thereof conjugated to said type V or type VI CRISPR/Cas effector protein in combination with said guide RNA.

16. The method of any one of claims 1-12, wherein the first target binding construct is an antibody or antigen binding fragment thereof conjugated to said trigger nucleic acid sequence.

17. The method of any one of claims 2-16, wherein the first and second target binding constructs bind the same antigen.

18. The method of any one of claims 2-16, wherein the first and second target binding constructs bind different antigens or epitopes of the same target.

19. The method of any one of claims 1-18, wherein the target is selected from a cell, cellular component or cell surface marker, a small molecule, a peptide, a polypeptide, and a cytokine.

20. The method of claim 19, wherein the target is a cytokine.

21. The method of claim 20, wherein the cytokine is IFN-gamma.

22. The method of any one of claims 1-18, wherein the target is a whole cell or whole microorganism.

23. The method of any one of claims 1-18, wherein said target is a Giardia cyst or Cryptosporidium oocyst.

24. The method of claim 23, wherein said target is a Cryptosporidium oocyst.

25. The method of any one of claims 22-24, wherein the first target binding is an anti-cryptosporidium antibody.

26. The method of any one of claim 19, or 22-25, wherein the target is detected at single cell or single organism sensitivity.

27. The method of any one of claims 1-21, wherein the target is detected at femtomolar sensitivity or lower.

28. The method of any one of claims 1-27, wherein the sample is a biological sample or an environmental sample.

29. The method of claim 28, wherein the biological sample is a blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate, or fluid obtained from a joint, or a swab of skin or mucosal membrane surface, a tissue biopsy, a culture of cells or medium from cell culture.

30. The method of claim 29, wherein the sample is blood, plasma, serum or a biopsy obtained from a human patient.

31. The method of claim 28, wherein the sample is a water sample.

32. The method of claim 28, wherein the sample is a crude sample.

33. The method of any of claims 1-31, wherein the sample is a concentrated or purified sample.

34. A method for the detection of a target in a sample, the method comprising:
(a) contacting the sample with:
(i) a first target binding construct to thereby immobilise or capture the target;
(ii) a second target binding construct;
(iii) a third binding construct which binds to the second target binding construct;
(iii) a type V or type VI CRISPR/Cas effector protein;
(iv) a trigger nucleic acid sequence;
(v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
(vi) a labelled reporter construct, wherein said reporter construct comprises a nucleic acid that does not hybridize with the guide sequence of the guide RNA and is cleavable by the nuclease activity of the activated type V or type VI CRISPR/Cas effector protein; and
(b) measuring a detectable signal produced following cleavage of the labelled nucleic acid reporter by the type V or type VI CRISPR/Cas effector protein, thereby detecting the immobilised or captured target;
wherein the type V or type VI CRISPR/Cas effector protein, the trigger nucleic acid sequence, the guide RNA, the type V or type VI CRISPR/Cas effector protein in combination with the guide RNA, or the type V or type VI CRISPR/Cas effector protein in combination with the guide RNA and the trigger nucleic acid is linked or conjugated to the third binding construct to thereby co-locate the type V or type VI CRISPR/Cas effector protein and the target when present in the sample; and wherein the target is not a nucleic acid sequence.

35. The method of claim 34, wherein the type V or type VI CRISPR/Cas effector protein is a Cas12a (Cpf1) or Cas12b (C2c1) protein, or Cas 13a or Cas 13b.

36. The method of claim 34 or 35, wherein the reporter construct is a labelled RNA.

37. The method of any one of claims 34-36, wherein the trigger nucleic acid sequence is a DNA sequence.

38. The method of any one of claims 34-37, wherein the first target binding construct is an antibody or antigen binding fragment thereof.

39. The method of any one of claims 34-38, wherein the first target binding construct is immobilised on a substrate or conjugated to a magnetic bead.

40. The method of claim 39, further comprising the step of performing magnetic separation of the captured target bound to the first target binding construct from the sample when the first target binding construct is conjugated to a magnetic bead.

41. The method of any one of claims 34-40, wherein the second target binding construct is an antibody or antigen binding fragment thereof.

42. The method of claim 41, wherein the antibody or antigen binding fragment thereof is labelled.

43. The method of any one of claims 34-42, wherein the third binding construct is an antibody or antigen binding fragment thereof conjugated to said type V or type VI CRISPR/Cas effector protein.

44. The method of any one of claims 34-42, wherein the third binding construct is an antibody or antigen binding fragment thereof conjugated to said guide RNA.

45. The method of any one of claims 34-42, wherein the third binding construct is an antibody or antigen binding fragment thereof conjugated to said type V or type VI CRISPR/Cas effector protein and said guide RNA.

46. The method of any one of claims 34-42, wherein the third binding construct is an antibody or antigen binding fragment thereof conjugated to said trigger nucleic acid sequence.

47. The method of any one of claims 34-46, wherein the first and second target binding constructs bind the same antigen.

48. The method of any one of claims 34-46, wherein the first and second target binding constructs bind different antigens or epitopes of the same target.

49. The method of any one of claims 34-48, wherein the target is selected from a cell, cellular component or cell surface marker, a small molecule, a peptide, a polypeptide, and a cytokine.

50. The method of claim 49, wherein the target is a cytokine.

51. The method of claim 50, wherein the cytokine is IFN-gamma.

52. The method of any one of claims 34-48, wherein the target is a whole cell or whole microorganism.

53. The method of any one of claims 34-48, wherein said target is a Giardia cyst or Cryptosporidium oocyst.

54. The method of claim 53, wherein said target is a Cryptosporidium oocyst.

55. The method of any one of claims 34-54, wherein the first target binding is an anti-cryptosporidium antibody.

56. The method of any one of claims 52-55, wherein the target is detected at single cell or single organism sensitivity.

57. The method of any one of claims 34-51, wherein the target is detected at femtomolar sensitivity.

58. The method of any one of claims 30-53, wherein the sample is a biological sample or an environmental sample.

59. The method of claim 58, wherein the biological sample is a blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate, or fluid obtained from a joint, or a swab of skin or mucosal membrane surface, a tissue biopsy, a culture of cells or medium from cell culture.

60. The method of claim 59, wherein the sample is blood, plasma, serum or a biopsy obtained from a human patient.

61. The method of claim 58, wherein the sample is a water sample.

62. The method of claim 58, wherein the sample is a crude sample.

63. The method of any of claims 34-61, wherein the sample is a concentrated or purified sample.

64. A kit for detecting a target in a sample, the kit comprising:
(i) a first binding construct;
(ii) a type V or type VI CRISPR/Cas effector protein;
(iii) a trigger nucleic acid sequence;
(iv) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
(v) a labelled reporter construct, wherein said reporter construct comprises a nucleic acid that is single stranded, does not hybridize with the guide sequence of the guide RNA and is cleavable by the nuclease activity of the activated type V or type VI CRISPR/Cas effector protein;
wherein the first binding construct binds to the target or to another agent which binds or attaches to the target, and wherein the type V or type VI CRISPR/Cas effector protein, the trigger nucleic acid sequence, the guide RNA, or the type V or type VI CRISPR/Cas effector protein in combination with the guide RNA, or the type V or type VI CRISPR/Cas effector protein in combination with the guide RNA and the trigger nucleic acid is linked or conjugated to the first binding construct to thereby co-locate the type V or type VI CRISPR/Cas effector protein and the target when present in the sample; and wherein the target is not a nucleic acid sequence.

65. A kit for detecting a target in a sample, the kit comprising:
(i) a target binding construct;
(ii) a second target binding construct;
(iii) a type V or type VI CRISPR/Cas effector protein;
(iv) a trigger nucleic acid sequence;
(v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and
(vi) a labelled reporter construct, wherein said reporter construct comprises a nucleic acid that is single stranded, does not hybridize with the guide sequence of the guide RNA and is cleavable by the nuclease activity of the activated type V or type VI CRISPR/Cas effector protein;
wherein the type V or type VI CRISPR/Cas effector protein, the trigger nucleic acid sequence, the guide RNA, or the type V or type VI CRISPR/Cas effector protein in combination with the guide RNA, or the type V or type VI CRISPR/Cas effector protein in combination with the guide RNA and the trigger nucleic acid is linked or conjugated to the second target binding construct so as to co-locate the type V or type VI CRISPR/Cas effector protein and the target when present in the sample; and
wherein the target is not a nucleic acid sequence.

66. The kit of claim 60 or 61, wherein the first target binding construct is immobilised on a substrate or conjugated to a magnetic bead.

67. The kit of any one of claims 60-62, further comprising one or more of: a reaction buffer, a washing buffer, and reagents for recovering or releasing immobilised or captured target.

68. The method according to claim 23 or 53, further comprising step of recovering Giardia cysts or Cryptosporidium oocysts by releasing captured cysts or oocysts from the first and/or second target binding agent, when a detectable signal is measured.

69. The method of claim 68, further comprising detecting the number of cysts or oocysts present in the sample by microscopy.

70. The method of any one of claims 1-63, 68 or 69, wherein the sample is also contacted with at least one sulfhydryl reductant.

71. The method of claim 70, wherein the sulfhydryl reductant is selected from the group consisting of Dithiothreitol (DTT), Tris(2-carboxyethyl) phosphine (TCEP) to and 2-Mercaptoethanol (2-ME)).

72. The method of claim 71, wherein the sulfhydryl reductant is DTT.

73. The method of claim 72, wherein said contacting occurs at a temperature of about 37° C.

74. The method of any one of claims 1-63, or 68-73, wherein the sample is also contacted with at least one non-ionic surfactant.

75. The method of claim 74, wherein the non-ionic surfactant is selected from the group consisting of Brij L23 and poly(vinyl alcohol) (PVA).

76. The method of claim 75, wherein the non-ionic surfactant is PVA.

77. The method of any one of claims 1-63, or 68-76, wherein said contacting occurs in a reaction mixture comprising a buffer.

78. A method of enhancing a type V or Type VI CRISPR/Cas detection system comprising adding a sulfhydryl reductant, and/or a non-ionic surfactant to a reaction mixture comprising the type V or Type VI CRISPR/Cas effector of the system.

79. The method of claim 78, comprising adding a sulfhydryl reductant and a non-ionic surfactant to a reaction mixture comprising the type V or Type VI CRISPR/Cas effector of the system.

80. The method of claim 78 or 79, wherein the sulfhydryl reductant is selected from the group consisting of Dithiothreitol (DTT), Tris(2-carboxyethyl) phosphine (TCEP) to and 2-Mercaptoethanol (2-ME)).

81. The method of claim 80, wherein the sulfhydryl reductant is DTT.

82. The method of claim 78 or 79, wherein the non-ionic surfactant is selected from the group consisting of Brij L23 and poly(vinyl alcohol) (PVA).

83. The method of claim 82, wherein the non-ionic surfactant is PVA.

84. The method of claim 79, wherein the wherein the sulfhydryl reductant is DTT and the non-ionic surfactant is PVA.

85. The kit of any one of claims 64-67, wherein said a reaction buffer, washing buffer, and reagents for recovering or releasing immobilised or captured target comprise a sulfhydryl reductant, and/or a non-ionic surfactant.

86. The kit of claim 85, wherein the sulfhydryl reductant is selected from the group consisting of Dithiothreitol (DTT), Tris(2-carboxyethyl) phosphine (TCEP) to and 2-Mercaptoethanol (2-ME)); and wherein the non-ionic surfactant is selected from the group consisting of Brij L23 and poly(vinyl alcohol) (PVA).

87. The kit of claim 86, wherein the sulfhydryl reductant is DTT and the non-ionic surfactant is PVA.

88. The kit of any one of claims 64-67, or 85-88, when used for a method for detecting a target in a sample.

89. A reaction mixture comprising: (i) at least one target binding construct; (ii) a type V or type VI CRISPR/Cas effector protein; (iii) a trigger nucleic acid sequence; (v) a guide RNA comprising: a region that binds to the type V or type VI CRISPR/Cas effector protein, and a guide sequence that hybridizes with the trigger nucleic acid sequence, wherein hybridization between the guide sequence and the trigger nucleic acid sequence activates the nuclease activity of the CRISPR/Cas effector protein; and (iv) a labelled reporter construct, wherein said reporter construct comprises a nucleic acid that does not hybridize with the guide sequence of the guide RNA and is cleavable by the nuclease activity of the activated type V or type VI CRISPR/Cas effector protein; wherein the type V or type VI CRISPR/Cas effector protein, the trigger nucleic acid, the guide RNA, or the type V or type VI CRISPR/Cas effector protein in combination with the guide RNA optionally in further combination with the trigger nucleic acid, is conjugated to said at least first target binding construct.

90. The reaction mixture of claim 89, further comprising a sample.

91. The reaction mixture of claim 89 or 90, further comprising one or more of: a reaction buffer.

92. The reaction mixture of claim 91, further comprising a sulfhydryl reductant, and/or a non-ionic surfactant.

93. The reaction mixture of claim 92, wherein the sulfhydryl reductant is selected from the group consisting of Dithiothreitol (DTT), Tris(2-carboxyethyl) phosphine (TCEP) to and 2-Mercaptoethanol (2-ME)), and wherein the non-ionic surfactant is selected from the group consisting of Brij L23 and poly(vinyl alcohol) (PVA).

94. The reaction mixture of claim 93, wherein the sulfhydryl reductant is DTT, and wherein the non-ionic surfactant is PVA.
